SEQUENCE LISTING (170 sequences)

SEQ ID NO: 1 - DNA, 10 bp, Artificial sequence
  Synthetic
cttttcattt

SEQ ID NO: 2 - DNA, 10 bp, Artificial sequence
  Synthetic
atggcgtttt

SEQ ID NO: 3 - DNA, 10 bp, Artificial sequence
  Synthetic
cgcgctgttc

SEQ ID NO: 4 - DNA, 10 bp, Artificial sequence
  Synthetic
gagacttcaa

SEQ ID NO: 5 - DNA, 10 bp, Artificial sequence
  Synthetic
gcgtggcttc

SEQ ID NO: 6 - DNA, 10 bp, Artificial sequence
  Synthetic
acggcgtttt

SEQ ID NO: 7 - DNA, 10 bp, Artificial sequence
  Synthetic
atatattttc

SEQ ID NO: 8 - DNA, 10 bp, Artificial sequence
  Synthetic
tcgtggtttc

SEQ ID NO: 9 - DNA, 10 bp, Artificial sequence
  Synthetic
gcggcggttc

SEQ ID NO: 10 - DNA, 10 bp, Artificial sequence
  Synthetic
ggagacttca

SEQ ID NO: 11 - DNA, 10 bp, Artificial sequence
  Synthetic
tttctaattc

SEQ ID NO: 12 - DNA, 10 bp, Artificial sequence
  Synthetic
agaacgattc

SEQ ID NO: 13 - DNA, 10 bp, Artificial sequence
  Synthetic
tgcagttttc

SEQ ID NO: 14 - DNA, 10 bp, Artificial sequence
  Synthetic
cgcgctcttc

SEQ ID NO: 15 - DNA, 10 bp, Artificial sequence
  Synthetic
aggcgatttc

SEQ ID NO: 16 - DNA, 10 bp, Artificial sequence
  Synthetic
aatcgtattc

SEQ ID NO: 17 - DNA, 10 bp, Artificial sequence
  Synthetic
aatcatattc

SEQ ID NO: 18 - DNA, 10 bp, Artificial sequence
  Synthetic
gtgttgaaac

SEQ ID NO: 19 - DNA, 34 bp, Artificial sequence
  Synthetic
catgaaaaaccctttttcgataaatacggcgttc

SEQ ID NO: 20 - DNA, 34 bp, Artificial sequence
  Synthetic
tatagattggttgtcattcacgatgcacgaagat

SEQ ID NO: 21 - DNA, 35 bp, Artificial sequence
  Synthetic
cttgtcttaatagtaacaactgcattgagaccgca

SEQ ID NO: 22 - DNA, 35 bp, Artificial sequence
  Synthetic
aaaaaaccataccgacccccacggcgcggcaggaa

SEQ ID NO: 23 - DNA, 34 bp, Artificial sequence
  Synthetic
catgaaaaaacctttttcgataaatacggcgttc

SEQ ID NO: 24 - DNA, 35 bp, Artificial sequence
  Synthetic
cgccagcagttcggtggcgcgttgctcaatacggg

SEQ ID NO: 25 - DNA, 34 bp, Artificial sequence
  Synthetic
tatagattggttgtcatttcagatgcacgaagat

SEQ ID NO: 26 - DNA, 34 bp, Artificial sequence
  Synthetic
agttgacggcgtatgccccgaacctaaaagcgta

SEQ ID NO: 27 - DNA, 35 bp, Artificial sequence
  Synthetic
ccgccacaaataccacccaccgcctcgctgcccac

SEQ ID NO: 28 - DNA, 34 bp, Artificial sequence
  Synthetic
agttgacggcgtatgccccgaacctagaagcgta

SEQ ID NO: 29 - DNA, 34 bp, Artificial sequence
  Synthetic
agacacacgcccgaagcatcaacggcttggaggc

SEQ ID NO: 30 - DNA, 35 bp, Artificial sequence
  Synthetic
aaaaaaccacaccgacccccacggcgcagcaggaa

SEQ ID NO: 31 - DNA, 35 bp, Artificial sequence
  Synthetic
gatacgggggcggtagtcgtcctctgttaaatagg

SEQ ID NO: 32 - DNA, 34 bp, Artificial sequence
  Synthetic
aacgaaatcaacaattatctcggcggctttggta

SEQ ID NO: 33 - DNA, 35 bp, Artificial sequence
  Synthetic
gtattgaagaaaccggttacatatacgcagcaaac

SEQ ID NO: 34 - DNA, 34 bp, Artificial sequence
  Synthetic
acccatgcaaagcagcttgtagcctatctaggac

SEQ ID NO: 35 - DNA, 34 bp, Artificial sequence
  Synthetic
cgtcttcgccgcgctgaaaaacgcttatttcgcc

SEQ ID NO: 36 - DNA, 34 bp, Artificial sequence
  Synthetic
ctgtcttaatagtaacaactgcattgagaccgca

SEQ ID NO: 37 - DNA, 34 bp, Artificial sequence
  Synthetic
agcgcagccgtagtcaacgcagcggcagccaagg

SEQ ID NO: 38 - DNA, 35 bp, Artificial sequence
  Synthetic
agtagtagagctgcccaatgcgttgccttgggagc

SEQ ID NO: 39 - DNA, 35 bp, Artificial sequence
  Synthetic
gtattgaagaaaccggttacagatacgcagcaaac

SEQ ID NO: 40 - DNA, 34 bp, Artificial sequence
  Synthetic
atcaagccccatatccaagaaacaataaaccact

SEQ ID NO: 41 - DNA, 34 bp, Artificial sequence
  Synthetic
agacgcacgcccgaagcatcaacggcttggaggc

SEQ ID NO: 42 - DNA, 10 bp, Artificial sequence
  Synthetic
gtgtaagtct

SEQ ID NO: 43 - DNA, 10 bp, Artificial sequence
  Synthetic
tccttgatga

SEQ ID NO: 44 - DNA, 10 bp, Artificial sequence
  Synthetic
aacagcggcg

SEQ ID NO: 45 - DNA, 10 bp, Artificial sequence
  Synthetic
gttgccgcgc

SEQ ID NO: 46 - DNA, 10 bp, Artificial sequence
  Synthetic
ctagctctaa

SEQ ID NO: 47 - DNA, 10 bp, Artificial sequence
  Synthetic
tctttgctga

SEQ ID NO: 48 - DNA, 9 bp, Artificial sequence
  Synthetic
gattcggca

SEQ ID NO: 49 - DNA, 10 bp, Artificial sequence
  Synthetic
cggcggatgt

SEQ ID NO: 50 - DNA, 10 bp, Artificial sequence
  Synthetic
gatttcggca

SEQ ID NO: 51 - DNA, 10 bp, Artificial sequence
  Synthetic
gcaatacacg

SEQ ID NO: 52 - DNA, 10 bp, Artificial sequence
  Synthetic
cggctttgcg

SEQ ID NO: 53 - DNA, 10 bp, Artificial sequence
  Synthetic
acacaatgct

SEQ ID NO: 54 - DNA, 10 bp, Artificial sequence
  Synthetic
gggaatggcg

SEQ ID NO: 55 - DNA, 10 bp, Artificial sequence
  Synthetic
ttgaccccaa

SEQ ID NO: 56 - DNA, 10 bp, Artificial sequence
  Synthetic
gcgccagcag

SEQ ID NO: 57 - DNA, 10 bp, Artificial sequence
  Synthetic
gagtaagtct

SEQ ID NO: 58 - DNA, 10 bp, Artificial sequence
  Synthetic
cagcaacagc

SEQ ID NO: 59 - DNA, 10 bp, Artificial sequence
  Synthetic
tgtctaaggg

SEQ ID NO: 60 - DNA, 10 bp, Artificial sequence
  Synthetic
ggaaacggcg

SEQ ID NO: 61 - DNA, 10 bp, Artificial sequence
  Synthetic
gatttcggaa

SEQ ID NO: 62 - DNA, 10 bp, Artificial sequence
  Synthetic
tcacacaact

SEQ ID NO: 63 - DNA, 10 bp, Artificial sequence
  Synthetic
tcagccgcct

SEQ ID NO: 64 - DNA, 10 bp, Artificial sequence
  Synthetic
tccttgctga

SEQ ID NO: 65 - DNA, 42 bp, Artificial sequence
  Synthetic
agggcgagggcgatgccacctacggcaagctgaccctgaagt

SEQ ID NO: 66 - DNA, 42 bp, Artificial sequence
  Synthetic
acttcagggtcagcttgccgtaggtggcatcgccctcgccct

SEQ ID NO: 67 - DNA, 42 bp, Artificial sequence
  Synthetic
agttcaagaccatctacatggccaagaagcccgtgcaactgc

SEQ ID NO: 68 - DNA, 42 bp, Artificial sequence
  Synthetic
gcagttgcacgggcttcttggccatgtagatggtcttgaact

SEQ ID NO: 69 - DNA, 42 bp, Artificial sequence
  Synthetic
agttaagggtttggggaagcactgggccaagagtcaggaaaa

SEQ ID NO: 70 - DNA, 42 bp, Artificial sequence
  Synthetic
ttttcctgactcttggcccagtgcttccccaaacccttaact

SEQ ID NO: 71 - DNA, 42 bp, Artificial sequence
  Synthetic
aggggcttcgctgggggagcctcggcttcttctgggagaaaa

SEQ ID NO: 72 - DNA, 42 bp, Artificial sequence
  Synthetic
ttttctcccagaagaagccgaggctcccccagcgaagcccct

SEQ ID NO: 73 - DNA, 42 bp, Artificial sequence
  Synthetic
aaaaacatgaaggagctaactatgggcctcgaccggaacaga

SEQ ID NO: 74 - protein, 14 aa, Artificial sequence
  Synthetic
KNMKELTMGLDRNR

SEQ ID NO: 75 - protein, 7 aa, Artificial sequence
  Synthetic
MGLDRNR

SEQ ID NO: 76 - DNA, 42 bp, Artificial sequence
  Synthetic
aaaaacatgaaagagctcactctgggcctcgaccggaacaga

SEQ ID NO: 77 - protein, 14 aa, Artificial sequence
  Synthetic
KNMKELTLGLDRNR

SEQ ID NO: 78 - DNA, 32 bp, Artificial sequence
  Synthetic
tcagccgcctctaggcggctgtgtgttgaaac

SEQ ID NO: 79 - DNA, 31 bp, Artificial sequence
  Synthetic
tcgcactcttcatgggtgcgtggattgaaat

SEQ ID NO: 80 - DNA, 32 bp, Artificial sequence
  Synthetic
gtcgccccccacgcgggggcgtggattgaaac

SEQ ID NO: 81 - DNA, 36 bp, Artificial sequence
  Synthetic
gtgtccaaaccattgatgccgtaaggcgttgagcac

SEQ ID NO: 82 - DNA, 37 bp, Artificial sequence
  Synthetic
ctttccttctactaatcccggcgatcgggactgaaac

SEQ ID NO: 83 - DNA, 42 bp, Artificial sequence
  Synthetic
acggcgtgcagtgcttcagccgctaccccgaccacatgaagc

SEQ ID NO: 84 - DNA, 41 bp, Artificial sequence
  Synthetic
gcttcatgtggtgcggggtagcggctgaagcactgcacgcc

SEQ ID NO: 85 - DNA, 26 bp, Artificial sequence
  Synthetic
cctcgtgaccaccctgacctacggcg

SEQ ID NO: 86 - DNA, 26 bp, Artificial sequence
  Synthetic
cgccgtaggtcagggtggtcacgagg

SEQ ID NO: 87 - DNA, 42 bp, Artificial sequence; identical to SEQ ID NO: 65
  Synthetic

SEQ ID NO: 88 - DNA, 42 bp, Artificial sequence; identical to SEQ ID NO: 66
  Synthetic

SEQ ID NO: 89 - DNA, 42 bp, Artificial sequence
  Synthetic
agttcgagggcgacaccctggtgaaccgcatcgagctgaagg

SEQ ID NO: 90 - DNA, 42 bp, Artificial sequence
  Synthetic
ccttcagctcgatgcggttcaccagggtgtcgccctcgaact

SEQ ID NO: 91 - DNA, 42 bp, Artificial sequence
  Synthetic
agttcgtgaccgccgccgggatcactctcggcatggacgagc

SEQ ID NO: 92 - DNA, 42 bp, Artificial sequence
  Synthetic
gctcgtccatgccgagagtgatcccggcggcggtcacgaact

SEQ ID NO: 93 - DNA, 42 bp, Artificial sequence
  Synthetic
gagttcgtgaccgccgccgggatcactctcggcatggacgag

SEQ ID NO: 94 - DNA, 42 bp, Artificial sequence
  Synthetic
ctcgtccatgccgagagtgatcccggcggcggtcacgaactc

SEQ ID NO: 95 - DNA, 40 bp, Artificial sequence
  Synthetic
gcatggcaccggcagcaccggcagcggcagctccggcacc

SEQ ID NO: 96 - DNA, 42 bp, Artificial sequence
  Synthetic
gcggtgccggagctgccgctgccggtgctgccggtgccatgc

SEQ ID NO: 97 - DNA, 54 bp, Artificial sequence
  Synthetic
gatgcccttcagctcgatgcggttcaccagggtgtcgccctcgaacttcacctc

SEQ ID NO: 98 - DNA, 54 bp, Artificial sequence
  Synthetic
gaggtgaagttcgagggcgacaccctggtgaaccgcatcgagctgaagggcatc

SEQ ID NO: 99 - DNA, 2505 bp, Artificial sequence
  Synthetic
gtgaatttcgactatatagcccacgctcgccaagactcatcaaaaaattggcattcccatcccctgcaaaaacatctacaaaaagtcgcccaactcgccaagcgttttgcagggcgttatgggtcgttgtttgccgaatatgcggggcttttgcacgatttggggaaatttcaggaatcttttcagaaatatatccgtaatgcatccggctttgaaaaagaaaatgcccatttggaagatgtcgaatctaccaagttgcgcaaaattccgcattccactgccggtgccaaatatgcggtagaacgtctaaatccatttttcgggcatttgctggcatatttgattgccgggcatcatgctgggctggcagattggtatgacaaaggcagcctgaaacgccgtctgcaacaggcggatgacgagttggcagcgtctttgtcgggctttgtggaaagtagtttgcccgaagattttttcccgttatcagatgatgacttgatgcgggatttttttgcgttttgggaagacggggcaaagctggaagaattgcatatttggatgcgttttctcttttcctgcttggtggatgccgattttttggataccgaagcctttatgaacggctatgccgatgcagatactgcgcaggctgccggattgcgcccaaaatttcccggtttggatgagttacaccggcgatatgagcaatatatggcgcaactttcagaaaaagcagataaaaattcatctttaaaccaagaacgccacgccattttgcagcaatgtttttctgccgcagaaacggaccgtactttgttttctttaaccgtgccgaccggtggcggtaaaactttggcgagcttgggcttcgctttgaagcacgcgctgaaatttggcaaaaaacgtattatctatgctattcctttcaccagtattatcgagcagaatgccaatgttttccgcaatgcattaggcgatgatgtggttttagaacaccacagcaatttggaagtgaaagaagataaggaaacagcgaaaactcgtcttgctacggaaaattgggacgcgccgctgattgttactaccaatgtgcaactgtttgaaagcctgtttgcggcgaaaaccagccgttgccgcaagattcacaatattgccgacagcgtggtgattttggatgaagcccagcagcttccgcgcgatttccaaaaaccgattaccgacatgatgcgggtgctggcgcgtgattacggcgttacctttgtgctgtgcacggcaacccaaccggagcttggcaaaaatatcgacgcattcggtcgcactattttggaagggctaccagatgtgcgcgaaattgtggcagacaaaattgccttatcggaaaaactgcgccgcgtccgcatcaaaatgccgccgccaaacggcgaaacgcaaagctggcagaaaattgccgatgaaatagccgcgcgcccgtgtgttttggcagtggtcaatacgcgaaaacacgcccaaaaactctttgccgccctgccttctaacggaatcaagctacatttatctgccaatatgtgcgccacacactgcagcgaagtgattgcgttggttcgccgatatttggcactgtatcgcgcaggcagcctgcacaagcccttgtggctggtcagcacgcagttgattgaagcaggcgtggatttggatttcccttgcgtgtatcgggcgatggcagggctggacagcattgcccaggcggcgggacggtgcaaccgtgaaggtaaactgccgcagttgggcgaagtagtcgtattccgcgccgaagaaggcgcgcccagcggcagcctgaaacaggggcaggacattaccgaagagatgctgaaagcagggctgcttgatgacccgctttccccgttggcatttgccgaatatttccgccgattcaacggcaaaggtgatgtggacaaacacggtatcacaacgcttttgacggcagaagcatcaaatgaaaatccgctggcaattaaattccgcacagctgccgaacgtttccacctgattgataaccaaggcgtggcactcattgtgccgtttatcccgttggctcattgggaaaaagacggcagtccgcaaatcgtcgaagcaaacgagctggacgattttttcagacgacatctagatggtgttgaagtttcagaatggcaggatattttggacaaacaacgctttccgcagccgccagacaactcctttgggcaaaccgatcaaccactgctgcccgagccgtttgaaagctggttcggtctgttggaaagcgacccgctcaaacacaaatgggtttaccgcaagctgcaacgctacacgattactgtgtacgaacacgaactgaaaaagttgcctgaacatgccgttttttcaagagcgggattgctcgtgttagataagggctattacaaagccgtgcttggcgcggattttgacgatgcggcttggctacctgaaaattcggttttatga

SEQ ID NO: 100 - DNA, 2583 bp, Artificial sequence
  Synthetic
atgaacttcgactatatcgcccacgccagacaggacagcagcaagaactggcactctcaccctctgcagaaacatctgcagaaggtggcccagctggccaagagatttgccggcagatacggcagcctgttcgccgaatatgccggcctgctgcacgatctgggcaagttccaagagagcttccagaagtacatccggaacgccagcggcttcgagaaagagaatgcccacctggaagatgtggaaagcaccaagctgcggaagatccctcactctacagccggcgctaagtacgccgtggaaagactgaaccccttcttcggccatctgctggcctatctgattgccggacatcatgccggactggccgattggtacgataagggcagcctgaagcggagactgcagcaagccgatgatgaactggccgcctctctgtccggcttcgtggaatcttctctgcccgaggacttcttccctctgtccgacgacgacctgatgagagacttcttcgccttctgggaggacggcgccaagctggaagaactgcacatctggatgcggtttctgttcagctgcctggtggacgccgacttcctggataccgaggccttcatgaacggctacgccgatgccgatacagcccaagctgctggactgaggcctaagttccctggcctggatgagctgcatcggagatacgagcagtacatggctcagctgtccgagaaggccgacaagaacagctccctgaatcaagagcggcacgccatcctgcagcagtgcttttctgccgccgagacagacagaaccctgttcagcctgacagtgcctacaggcggcggaaaaactctggcctctctgggctttgccctgaagcacgccctgaagttcggcaagaagcggatcatctacgccattcctttcaccagcatcatcgagcagaacgccaacgtgttcagaaacgccctgggcgacgatgtggtgctggaacaccacagcaacctggaagtgaaagaggacaaagagacagccaagaccagactggccaccgagaattgggatgcccctctgatcgtgaccaccaacgtgcagctgttcgagagcctgtttgccgccaagacctccagatgcagaaagatccacaatatcgccgacagcgtggtcatcctggacgaagctcagcagctgccccgggacttccagaaacctatcaccgatatgatgcgcgtgctggccagagactacggcgtgacctttgtgctgtgtaccgccacacagcctgagctgggcaagaacatcgatgccttcggccggaccatcctggaaggattgcctgacgtgcgggaaatcgtggccgataagatcgccctgagcgagaagctgagaagagtgcggatcaagatgcctcctccaaacggcgagacacagagctggcagaagatcgccgacgagatcgccgctagaccatgtgtgctggccgtggtcaacaccagaaaacacgcccagaagctgttcgctgccctgcctagcaatggcatcaagctgcacctgagcgccaacatgtgcgccacacactgctctgaagtgatcgccctcgtgcggagatatctggccctgtacagagccggaagcctgcacaaacctctgtggctggtgtctacccagctgattgaagctggcgtggacctggacttcccctgtgtgtatagagccatggccggcctggattctattgcccaagcagccggacggtgcaacagagagggaaaactgcctcagctgggcgaagtggtggtgttcagagctgaagaaggcgcccctagcggctctctgaagcaaggccaggatatcaccgaggaaatgctgaaggccggactgctggacgaccctttgtctcctctggccttcgccgagtacttcagacggttcaatggcaagggcgacgtggacaagcacggcatcacaacactgctgacagccgaggccagcaacgagaatccactggccatcaagttccggaccgccgctgagagattccacctgatcgataatcagggcgtcgcactgatcgtgcccttcattcctctggctcactgggagaaagacggcagccctcagatcgtggaagccaacgagctggacgatttcttcaggcggcacctggacggcgtggaagtgtctgagtggcaggacatcctggataagcagcggttccctcagcctcctgacaacagctttggccagaccgatcagcctctgctgcctgagcctttcgagagttggttcggcctgctcgagagcgacccactgaagcacaaatgggtgtaccggaagctgcagcggtacaccatcaccgtgtatgagcacgagctgaaaaagctgcccgagcacgccgtgttctctagagctggactgctcgtgctggacaagggctactataaggccgtgctgggcgccgattttgacgatgctgcttggctgccagagaactctgtgctgggctctgtgggctacccctacgatgtgcctgattacgccggcagctaccctgagttccccaagaaaaagcggaaagtgtga

SEQ ID NO: 101 - protein, 834 aa, Neisseria lactamica
MNFDYIAHARQDSSKNWHSHPLQKHLQKVAQLAKRFAGRYGSLFAEYAGLLHDLGKFQESFQKYIRNASGFEKENAHLEDVESTKLRKIPHSTAGAKYAVERLNPFFGHLLAYLIAGHHAGLADWYDKGSLKRRLQQADDELAASLSGFVESSLPEDFFPLSDDDLMRDFFAFWEDGAKLEELHIWMRFLFSCLVDADFLDTEAFMNGYADADTAQAAGLRPKFPGLDELHRRYEQYMAQLSEKADKNSSLNQERHAILQQCFSAAETDRTLFSLTVPTGGGKTLASLGFALKHALKFGKKRIIYAIPFTSIIEQNANVFRNALGDDVVLEHHSNLEVKEDKETAKTRLATENWDAPLIVTTNVQLFESLFAAKTSRCRKIHNIADSVVILDEAQQLPRDFQKPITDMMRVLARDYGVTFVLCTATQPELGKNIDAFGRTILEGLPDVREIVADKIALSEKLRRVRIKMPPPNGETQSWQKIADEIAARPCVLAVVNTRKHAQKLFAALPSNGIKLHLSANMCATHCSEVIALVRRYLALYRAGSLHKPLWLVSTQLIEAGVDLDFPCVYRAMAGLDSIAQAAGRCNREGKLPQLGEVVVFRAEEGAPSGSLKQGQDITEEMLKAGLLDDPLSPLAFAEYFRRFNGKGDVDKHGITTLLTAEASNENPLAIKFRTAAERFHLIDNQGVALIVPFIPLAHWEKDGSPQIVEANELDDFFRRHLDGVEVSEWQDILDKQRFPQPPDNSFGQTDQPLLPEPFESWFGLLESDPLKHKWVYRKLQRYTITVYEHELKKLPEHAVFSRAGLLVLDKGYYKAVLGADFDDAAWLPENSVL

SEQ ID NO: 102 - DNA, 645 bp, Artificial sequence
  Synthetic
atgaggttcatcctggaaatcagtggtgatttggcatgcttcacaaggtctgagctaaaggtggaaagggttagttatcctgtgataacgccgtctgccgccaggaacatcctaatggcgatattgtggaagccggcgattcgctggaaggtcttgaagatagaaatcctaaaaccgattcagtggacgaatatccgccgcaacgaagtgggaactaagatgagtgagcgtagcggctcgctctatattgaagataaccgccagcagcgcgcatccatgctgctgaaagacgttgcctaccgcattcacgccgattttgacatgaccagtgaagcgggcgagagcgacaactatgttaaatttgccgaaatgttcaagcggcgggcaaagaaaggacaatatttccaccaaccttatttaggctgtcgtgagtttccttgtgatttcaggttgctggaaaaagccgaagatggattgccactcgaagacattacccaagatttcggttttatgctgtatgacatggatttcagcaaatccgacccgcgtgattccaataacgccgagccgatgttttaccaatgcaaagcggtaaacggcgtgattaccgtgccgcctgccgacagcgaggaggtgaaacgatga

SEQ ID NO: 103 - DNA, 705 bp, Artificial sequence
  Synthetic
atgcggttcatcctggaaatcagcggcgacctggcctgcttcacaagaagcgagctgaaggtcgagcgggtgtcataccctgtgatcacccctagcgccgccagaaacatcctgatggccattctgtggaagcccgccatcagatggaaggtgctgaagatcgagatcctgaagcctatccagtggaccaacatccggcggaacgaagtgggcaccaagatgagcgagagaagcggcagcctgtacatcgaggacaacagacagcagcgggcctccatgctgctgaaggatgtggcctatagaatccacgccgacttcgacatgacaagcgaggccggcgagagcgacaactacgtgaagttcgccgagatgttcaagcggagagccaagaagggccagtacttccaccagccttacctgggctgcagagagttcccctgcgacttcagactgctggaaaaggccgaggatggcctgcctctggaagatatcacccaggacttcggcttcatgctgtacgacatggacttcagcaagagcgaccccagagacagcaacaacgccgagcctatgttctaccagtgcaaggccgtgaacggcgtgatcactgtgcctccagccgatagcgaggaagtgaagagaggcagcgtcggctacccctacgatgtgcctgattacgcccctaagaaaaagcggaaagtgtga

SEQ ID NO: 104 - protein, 214 aa, Neisseria lactamica
MRFILEISGDLACFTRSELKVERVSYPVITPSAARNILMAILWKPAIRWKVLKIEILKPIQWTNIRRNEVGTKMSERSGSLYIEDNRQQRASMLLKDVAYRIHADFDMTSEAGESDNYVKFAEMFKRRAKKGQYFHQPYLGCREFPCDFRLLEKAEDGLPLEDITQDFGFMLYDMDFSKSDPRDSNNAEPMFYQCKAVNGVITVPPADSEEVKR

SEQ ID NO: 105 - DNA, 1758 bp, Artificial sequence
  Synthetic
atgattttgcacgcgctcacccaatactatcaacgcaaagccgaaagtgatggcggtattgcccaggaagggtttgaaaacaaagaaataccgttcattatcgttatagacaaacagggtaattttattcagctggaagatacccgtgagctgaaagttaagaagaaagttggccgcacttttttagtaccgaaaggtttgggcaggagcggttcaaaatcctacgaagtaagcaatttattgtgggatcactacggttatgtacttgcttatgccggagaaaaagggcaggagcaggcggacaaacagcatgccagctttaccgccaaagtaaatgaattgaaacaggcgctgcccgatgatgcaggtgttactgcggttgctgcctttttgtcttctgcggaagaaaaaagcaaagtcatgcaggctgcaaattgggcggagtgtgccaaagtcaaaggctgtaatctcagcttccgcctggtggatgaagcggtagatttggtttgccagtcaaaggcggtgcgggaatatgtgagtcaagcaaatcaaacgcaatccgataatgtccaaaaaggcatttgcctggtaacgggcaaagctgcgccgattgcgcggctgcataacgccgtgaaaggcgtgaatgccaagcccgccccgtttgcatcggtaaatctgtcggcttttgaatcatacggcaaagagcagggctttatctttcccgtgggcgagcaagccatgttcgaatataccaccgccttgaacaccttgcttgctagcgaaaaccgattccgtatcggcgatgtaacggccgtatgttggggcgcgaaacggactccgttggaggaaagtcttgcttcgatgattaacggcggcggcaaagacaagcccgatgagcatatcgatgccgttaaaactctttataaaagcctatacaacggtcaataccaaaaacctgacggcaaagaaaaattctaccttttaggtttatcgcccaattccgcgcgcattgtcgtccgcttttggcatgaaaccaccgttgccgccttatcagaaagtattgcggcgtggtatgacgatttgcaaatggtgcgcggcgaaaactcgccataccccgaatatatgccgctaccgcgcctgctgggtaatttggtgttggacggcaaaatggaaaacctgccatctgacctgattgcccaaataaccgatgccgcgctcaacaaccgtgttttacccgtcagcctgttgcaggctgctttgcggcgcaacaaggcggaacagaaaattacctatggcagagcaagtctgcttaaagcctatatcaatcgcgcaatccgtgcgggtcgtctgaaaaacatgaaggagctaactatgggcctagatagaaaccgtcaagacatcggctatgtgctggggcggctgtttgccgtgctggaaaaaatacaagccgaggccaatcccggcttgaacgccaccattgccgaccgctatttcggttcggcaagcagcacaccgattgccgtattcggcacactgatgcgcttgttgccgcaccatttgaacaaactggaatttgaaggacgtgccgtacaactgcaatgggaaatccgccagattttggaacattgtcagagatttcctaaccatttgaatttggaacagcaaggcctatttgccatcggttactaccacgaaacccaattcctgtttaccaaagacgcattgaaaaacctgttcaacgaagcgaaaaccgcataa

SEQ ID NO: 106 - DNA, 1821 bp, Artificial sequence
  Synthetic
atggtgcccaagaaaaagcggaaggtgtacccctacgacgtgcccgattatgccggctctgtgggaattctgcacgccctgacacagtactaccagcggaaggccgaaagcgacggcggaattgcccaagagggcttcgagaacaaagagatccccttcatcatcgtgatcgacaagcagggcaacttcatccagctcgaggacacccgcgagctgaaagtgaagaaaaaagtgggccgcaccttcctggtgcctaaaggccttggcagaagcggcagcaagagctacgaggtgtccaacctgctgtgggaccactacggatacgtgctggcctatgccggcgagaagggacaagaacaggccgataagcagcacgccagcttcaccgccaaagtgaacgagctgaagcaggccctgcctgatgatgctggcgtgacagctgtggccgcctttctgtctagcgccgaagagaagtccaaagtgatgcaggccgccaactgggccgagtgcgctaaagtgaagggctgcaacctgagcttccggctggtggatgaagccgtggatctcgtgtgtcagtctaaggccgtgcgcgagtatgtgtcccaggccaatcagacccagagcgacaacgtgcagaaaggcatctgtctggtcaccggcaaggccgctcctattgccagactgcacaatgccgtgaagggcgtgaacgccaagcctgctcctttcgcctctgtgaacctgagcgcctttgagagctacggcaaagagcagggcttcatcttccctgtgggagagcaggccatgttcgagtacaccaccgctctgaataccctgctggcctccgagaacagattccggatcggagatgtgaccgccgtgtgttggggagccaagagaacacctctggaagagtccctggccagcatgatcaatggcggcggaaaggacaagcccgacgagcacatcgacgccgtgaaaaccctgtacaagagcctgtacaacggccagtaccagaagcctgacggaaaagagaagttctacctgctgggactgagccccaacagcgccagaatcgttgtgcggttctggcacgagacaaccgtggctgccctgtctgagtctatcgccgcttggtacgacgacctgcagatggttcgaggcgagaacagcccctatcctgagtacatgcccctgcctagactgctgggcaacctggtgctggacggcaagatggaaaacctgcctagcgacctgatcgcccagatcacagatgctgccctgaacaacagagtgctgcctgtcagtctgctgcaggcagccctgagaagaaacaaggccgagcagaagatcacctacggcagagccagcctgctgaaggcctacatcaaccgggccatcagagccggacggctgaagaacatgaaggaactgaccatgggcctcgaccggaacagacaggatatcggctatgtgctgggcagactgttcgccgtgctggaaaagattcaggccgaggccaatcctggcctgaacgccacaatcgccgacagatattttggcagcgccagcagcacacctatcgccgtgtttggcaccctgatgagactgctgcctcaccacctgaacaagctggaattcgagggcagagccgtgcagctccagtgggagatcagacagatcctggaacactgccagcggttccccaatcacctgaacctggaacagcagggactgtttgccatcggctactaccacgagacacagtttctgttcaccaaggacgccctgaagaacctgttcaacgaggccaagaccgcctga

SEQ ID NO: 107 - protein, 585 aa, Neisseria lactamica
MILHALTQYYQRKAESDGGIAQEGFENKEIPFIIVIDKQGNFIQLEDTRELKVKKKVGRTFLVPKGLGRSGSKSYEVSNLLWDHYGYVLAYAGEKGQEQADKQHASFTAKVNELKQALPDDAGVTAVAAFLSSAEEKSKVMQAANWAECAKVKGCNLSFRLVDEAVDLVCQSKAVREYVSQANQTQSDNVQKGICLVTGKAAPIARLHNAVKGVNAKPAPFASVNLSAFESYGKEQGFIFPVGEQAMFEYTTALNTLLASENRFRIGDVTAVCWGAKRTPLEESLASMINGGGKDKPDEHIDAVKTLYKSLYNGQYQKPDGKEKFYLLGLSPNSARIVVRFWHETTVAALSESIAAWYDDLQMVRGENSPYPEYMPLPRLLGNLVLDGKMENLPSDLIAQITDAALNNRVLPVSLLQAALRRNKAEQKITYGRASLLKAYINRAIRAGRLKNMKELTMGLDRNRQDIGYVLGRLFAVLEKIQAEANPGLNATIADRYFGSASSTPIAVFGTLMRLLPHHLNKLEFEGRAVQLQWEIRQILEHCQRFPNHLNLEQQGLFAIGYYHETQFLFTKDALKNLFNEAKTA

SEQ ID NO: 108 - DNA, 849 bp, Artificial sequence
  Synthetic
atgactattgaaaaacgctacgactttgtctttttatttgatgtgcaagacggcaatcccaacggcgatcctgacgcaggtaacctgccgcgtatcgacccgcaaaccggcgaaggtttggtaactgatgtttgcctgaaacgcaaagtccgcaactttatccaaatgactcaaaatgacgaacatcacgacatctttatccgcgaaaaaggcattttgaacaacctgattgacgaagcccacgagcaggaaaacgtaaaaggcaaagaaaaaggcgagaaaaccgaagctgcccgccaatacatgtgcagccgttattacgacatccgcacatttggcgcagtgatgactaccggcaaaaatgcaggacaagtacgcggtcccgtgcaactgactttttctcgctctattgatcccatcatgaccttggaacacagcattacccgcatggcggttaccaacgaaaaagatgccagtgaaaccggcgacaaccgtacaatgggtcgcaaattcaccgtcccctacggtctataccgctgccatggcttcatttctacccattttgccaaacaaacaggcttttccgaaaacgatttagagctgttttggcaggcacttgtcaatatgtttgaccacgaccattccgccgcacgcggacaaatgaacgcacgcgggctctatgtgtttgaacacagcaataatctaggtgatgcgcctgctgatagtctgttcaaacgcattcaggtagtcaaaaaggacggtgtagaagtagtaaggagttttgacgattatcttgtcagcgtagacgataagaatcttgaagaaaccaagctgttgcgtaaattaggc

SEQ ID NO: 109 - DNA, 912 bp, Artificial sequence
  Synthetic
atgaccatcgagaagcgctacgacttcgtgttcctgttcgacgtgcaagacggcaaccccaacggcgatcctgatgccggaaacctgcctagaatcgaccctcagacaggcgagggcctcgtgacagatgtgtgcctgaagcggaaagtgcggaacttcatccagatgacccagaacgacgagcaccacgacatcttcatcagagagaagggcatcctgaacaacctgatcgacgaggcccacgagcaagagaacgtgaagggcaaagagaaaggcgagaaaaccgaggccgccagacagtacatgtgcagccggtactacgacatcagaaccttcggcgccgtgatgaccaccggcaagaatgctggacaagtgcggggacctgtgcagctgaccttcagcagatccatcgatcccatcatgaccctggaacacagcatcaccagaatggccgtgaccaatgagaaggacgccagcgaaaccggcgacaacagaaccatgggcagaaagttcaccgtgccttacggcctgtaccggtgccacggctttatcagcacccacttcgccaagcagaccggcttcagcgagaacgacctggaactgttttggcaggccctggtcaacatgttcgatcacgatcactctgccgccagaggccagatgaatgccagaggactgtacgtgttcgagcacagcaacaacctgggagatgcccctgccgacagcctgttcaagagaatccaggtggtcaagaaagacggcgtggaagtcgtgcggagcttcgacgattacctggtgtccgtggacgacaagaacctggaagagacaaagctgctgcggaagctcggcggctctgtgggctatccttacgacgtgccagactacgcccctaagaaaaagcgcaaagtgtga

SEQ ID NO: 110 - protein, 283 aa, Neisseria lactamica
MTIEKRYDFVFLFDVQDGNPNGDPDAGNLPRIDPQTGEGLVTDVCLKRKVRNFIQMTQNDEHHDIFIREKGILNNLIDEAHEQENVKGKEKGEKTEAARQYMCSRYYDIRTFGAVMTTGKNAGQVRGPVQLTFSRSIDPIMTLEHSITRMAVTNEKDASETGDNRTMGRKFTVPYGLYRCHGFISTHFAKQTGFSENDLELFWQALVNMFDHDHSAARGQMNARGLYVFEHSNNLGDAPADSLFKRIQVVKKDGVEVVRSFDDYLVSVDDKNLEETKLLRKLG

SEQ ID NO: 111 - DNA, 387 bp, Artificial sequence
  Synthetic
atgggcctagatagaaaccgtcaagacatcggctatgtgctggggcggctgtttgccgtgctggaaaaaatacaagccgaggccaatcccggcttgaacgccaccattgccgaccgctatttcggttcggcaagcagcacaccgattgccgtattcggcacactgatgcgcttgttgccgcaccatttgaacaaactggaatttgaaggacgtgccgtacaactgcaatgggaaatccgccagattttggaacattgtcagagatttcctaaccatttgaatttggaacagcaaggcctatttgccatcggttactaccacgaaacccaattcctgtttaccaaagacgcattgaaaaacctgttcaacgaagcgaaaaccgcataa

SEQ ID NO: 112 - DNA, 450 bp, Artificial sequence
  Synthetic
atggtgcccaagaaaaagcggaaggtgtacccctacgacgtgcccgattatgccggctctgtgggaggcctcgaccggaacagacaggatatcggctatgtgctgggcagactgttcgccgtgctggaaaagattcaggccgaggccaatcctggcctgaacgccacaatcgccgacagatattttggcagcgccagcagcacacctatcgccgtgtttggcaccctgatgagactgctgcctcaccacctgaacaagctggaattcgagggcagagccgtgcagctccagtgggagatcagacagatcctggaacactgccagcggttccccaatcacctgaacctggaacagcagggactgtttgccatcggctactaccacgagacacagtttctgttcaccaaggacgccctgaagaacctgttcaacgaggccaagaccgcctga

SEQ ID NO: 113 - protein, 128 aa, Neisseria lactamica
MGLDRNRQDIGYVLGRLFAVLEKIQAEANPGLNATIADRYFGSASSTPIAVFGTLMRLLPHHLNKLEFEGRAVQLQWEIRQILEHCQRFPNHLNLEQQGLFAIGYYHETQFLFTKDALKNLFNEAKTA

SEQ ID NO: 114 - DNA, 32 bp, Artificial sequence; identical to SEQ ID NO: 78
  Synthetic

SEQ ID NO: 115 - DNA, 35 bp, Artificial sequence
  Synthetic
gagggcgacaccctggtgaaccgcatcgagctgaa

SEQ ID NO: 116 - DNA, 35 bp, Artificial sequence
  Synthetic
aagaccatctacatggccaagaagcccgtgcaact

SEQ ID NO: 117 - DNA, 35 bp, Artificial sequence
  Synthetic
ctgactcttggcccagtgcttccccaaacccttaa

SEQ ID NO: 118 - DNA, 35 bp, Artificial sequence
  Synthetic
ttactgtccccttctgggctcactatgctgccgcc

SEQ ID NO: 119 - protein, 221 aa, Synechocystis sp. PCC6803
MNFIDLAFPVKGTVLNADHNYYLYSAIAKEFPILHDLPDLAVNTISGKPDREGKILLVPGSKLWMRLPIDNITHIYQLAGKKLRIGQYSIELGNPSLHPLEPVESLKARIITIKGHTEPISFLEAVKRQLFALEITEGDVGIPANHEGIPKRLTLQIKKPERTYSIVGYSVLLSNLSAEDSLKIQQVGIGGKRRLGCGVFYPAVKKSTNSGNKKNVEATLG

SEQ ID NO: 120 - DNA, 732 bp, Artificial sequence
  Synthetic
atggtgcccaagaaaaagcggaaggtgtacccctacgacgtgcccgattatgccggctctgtgggaatgaacttcatcgacctggctttccccgtgaagggcaccgtgctgaacgccgaccacaactactacctgtacagcgccattgccaaagagttccccatcctgcacgacctgcctgacctggccgtgaataccatcagcggcaagcctgacagagagggcaagatcctgctggtgcctggcagcaagctgtggatgagactgcccatcgacaacatcacccacatctaccagctggccggcaagaagctgagaatcggccagtactctatcgagctgggcaacccctctctgcaccctctggaacctgtggaaagcctgaaggcccggatcatcaccatcaagggccataccgagcctatcagcttcctggaagccgtgaagagacagctgttcgccctggaaatcaccgagggcgacgtgggaattcctgccaatcacgagggcatccccaagagactgaccctgcagatcaagaagcccgagcggacctatagcatcgtgggctactctgtgctgctgagcaatctgagcgccgaggacagcctgaagatccagcaagttggcatcggcggcaagagaaggcttggctgtggcgtgttctaccccgccgtgaaaaagagcaccaactccggcaacaagaagaacgtcgaggccacactgggctga

SEQ ID NO: 121 - protein, 551 aa, Synechocystis sp. PCC6803
MPKTQAEILTLDFNLAELPSAQHRAGLAGLILMIRELKKWPWFKIRQKEKDVLLSIENLDQYGASIQLNLEGLIALFDLAYLSFTEERKSKSKIKDFKRVDEIEIEENGKNKIQKYYFYDVITPQGGFLAGWDKSDGQIWLRIWRDMFWSIIKGVPATRNPFNNRCGLNLNAGDSFSKDVESVWKSLQNAEKTTGQSGAFYLGAMAVNAENVSTDDLIKWQFLLHFWAFVAQVYCPYILDKDGKRNFNGYVIVIPDIANLEDFCDILPDVLSNRNSKAFGFRPQESVIDVPEQGALELLNLIKQRIAKKAGSGLLSDLIVGVEVIHAEKQGNSIKLHSVSYLQPNEESVDDYNAIKNSYYCPWFRRQLLLNLVNPKFDLASQSWLKRHPWYGFGDLLSRIPQRWLKENNSYFSHDARQLFTQKGDFDMTVATTKTREYAEIVYKIAQGFVLSKLSSKHDLQWSKCKGNPKLEREYNDKKEKVVNEAFLAIRSRTEKQAFIDYFVSTLYPHVRQDEFVDFAQKLFQDTDEIRSLTLLALSSQYPIKRQGETE

SEQ ID NO: 122 - DNA, 1722 bp, Artificial sequence
  Synthetic
atggtgcccaagaaaaagcggaaggtgtacccctacgacgtgcccgattatgccggctctgtgggaatgcctaagacacaggccgagatcctgacactggacttcaacctggccgagctgcctagcgctcagcatagagctggactggccggactgatcctgatgatccgcgagctgaagaagtggccctggttcaagatccggcagaaagaaaaggacgtgctgctgagcatcgagaacctggatcagtacggcgccagcatccagctgaatctggaaggactgatcgccctgttcgacctggcctacctgagcttcaccgaggaacggaagtccaagagcaagatcaaggacttcaagcgcgtggacgagatcgagatcgaagagaacggcaagaacaagatccagaagtactacttttacgacgtgataacccctcaaggcggcttcctggccggctgggataagtctgatggacagatctggctgcggatctggcgggacatgttctggtccatcatcaagggcgtgcccgccaccagaaatcccttcaacaatagatgcggcctgaacctgaacgccggcgacagctttagcaaggacgtggaaagcgtgtggaagtccctgcagaacgccgagaaaaccacaggacagagcggcgccttttacctgggagccatggccgtgaatgccgagaacgtgtccaccgacgacctgatcaagtggcagttcctgctgcacttctgggccttcgtggcccaggtgtactgcccctacatcctggacaaggacggcaagcggaacttcaacggctacgtgatcgtgatccccgatatcgccaacctggaagatttctgcgacatcctgcctgacgtgctgagcaacagaaacagcaaggccttcggcttcagaccccaagagtccgtgatcgatgtgcctgagcaaggcgctctggaactgctgaacctcatcaagcagcggatcgccaagaaggccggaagcggactgctgagcgatctgatcgtgggcgtcgaagtgatccacgccgaaaagcagggcaacagcatcaagctgcacagcgtgtcctacctgcagcctaacgaagagtctgtggacgactacaacgccatcaagaacagctactactgcccatggttccggcggcagctgctgctgaatctcgtgaaccccaagttcgatctggccagccagagctggctgaagagacacccttggtacggcttcggcgacctgctgtctagaatccctcagcggtggctgaaagagaacaactcctacttcagccacgacgcccggcagctgtttacccagaaaggcgacttcgacatgaccgtggccaccaccaagaccagagaatacgccgagatcgtgtacaagatcgcccagggcttcgtgctgtccaagctgagcagcaagcacgacctgcagtggtccaagtgcaagggcaaccccaagctggaaagagagtacaacgacaaaaaagagaaggtcgtcaacgaggccttcctggctatcagaagccggacagagaagcaggccttcatcgactacttcgtgtccacactgtaccctcacgtgcggcaggacgagttcgtggattttgcccagaagctgttccaggacaccgacgaaatcagatctctgaccctgctggccctgtcctctcagtaccctatcaagagacagggcgagacagagtga

SEQ ID NO: 123 - protein, 301 aa, Synechocystis sp. PCC6803
MSNLNLFATILTYPAPASNYRGESEENRSVIQKILKDGQKYAIISPESMRNALREMLIELGQPNNRTRLHSEDQLAVEFKEYPNPDKFADDFLFGYMVAQTNDAKEMKKLNRPAKRDSIFRCNMAVAVNPYKYDTVFYQSPLNAGDSAWKNSTSSALLHREVTHTAFQYPFALAGKDCAAKPEWVKALLQAIAELNGVAGGHARAYYEFAPRSVVARLTPKLVAGYQTYGFDAEGNWLELSRLTATDSDNLDLPANEFWLGGELVRKMDQEQKAQLEAMGAHLYANPEKLFADLADSFLGV

SEQ ID NO: 124 - DNA, 966 bp, Artificial sequence
  Synthetic
atgagcaacctgaacctgttcgccaccatcctgacataccctgctccagccagcaactacagaggcgagagcgaagagaacagaagcgtgatccagaagatcctgaaggacggccagaagtacgccatcatcagccccgagagcatgcggaatgccctgagagagatgctgatcgagctgggccagcctaacaaccggacaagactgcacagcgaggatcagctggccgtggaattcaaagagtaccccaatcctgacaagttcgccgacgacttcctgttcggctacatggtggcccagaccaacgacgccaaagagatgaagaagctgaacagacccgccaagcgggacagcatcttcagatgcaatatggccgtggccgtgaatccctataagtacgacaccgtgttctatcagagccctctgaacgccggcgatagcgcctggaagaatagcacatctagcgccctgctgcaccgggaagtgacccatacagcctttcagtaccccttcgctctggccggcaaagactgtgccgctaagcctgaatgggtcaaagctctgctgcaggccattgccgagctgaatggtgttgctggcggacacgccagagcctactatgagtttgcccctagaagcgtggtggcccggctgacacctaaactggtggccggctatcagacctacggcttcgatgccgaaggcaactggctggaactgagcagactgaccgccaccgacagcgacaatctggacctgcctgccaacgagttttggctcggcggcgaactcgtgcggaagatggatcaagagcagaaggcccagctggaagccatgggagcccacctgtatgccaatccagagaagctgttcgccgatctggccgactctttcctgggcgtgggcagcgtcggctacccctacgatgtgcctgattacgcccctaagaaaaagcggaaagtgtga

SEQ ID NO: 125 - protein, 237 aa, Synechocystis sp. PCC6803
MAQLALALDTVTRYLRLKAPFAAFRPFQSGSFRSTTPVPSFSAVYGLLLNLAGIEQRQEVEGKVTLIKPKAELPKLAIAIGQVKPSSTSLINQQLHNYPVGNSGKEFASRTFGSKYWIAPVRREVLVNLDLIIGLQSPVEFWQKLDQGLKGETVINRYGLPFAGDNNFLFDEIYPIEKPDLASWYCPLEPDTRPNQGACRLTLWIDRENNTQTTIKVFSPSDFRLEPPAKAWQQLPG

SEQ ID NO: 126 - DNA, 774 bp, Artificial sequence
  Synthetic
atggctcaactggccctggctctggataccgtgaccagatacctgagactgaaggcccctttcgccgccttcagaccttttcagagcggcagcttccggtccaccacacctgtgccatctttcagcgccgtgtatggcctgctgctgaatctggccggaatcgagcagcggcaagaggtggaaggcaaagtgaccctgatcaagcccaaggccgagctgcctaaactggccattgccatcggccaagtgaagcccagcagcaccagcctgatcaaccagcagctgcacaactaccccgtgggcaacagcggcaaagagttcgccagcagaaccttcggcagcaagtactggatcgcccctgtgcggagagaggtgctggtcaacctggatctgatcatcggcctgcagagccccgtggaattctggcagaaactggaccagggcctgaagggcgagacagtgatcaacagatacggcctgccttttgccggcgacaacaacttcctgttcgacgagatctaccccatcgagaagcccgatctggccagctggtactgccctctggaacccgacaccagacctaatcagggcgcctgtagactgaccctgtggatcgacagagagaacaacacccagaccaccatcaaggtgttcagccccagcgacttccggctggaacctcctgctaaagcttggcagcagctccctggaggcagcgtcggctacccctacgatgtgcctgattacgcccctaagaaaaagcggaaagtgtga

SEQ ID NO: 127 - protein, 124 aa, Synechocystis sp. PCC6803
MTVATTKTREYAEIVYKIAQGFVLSKLSSKHDLQWSKCKGNPKLEREYNDKKEKVVNEAFLAIRSRTEKQAFIDYFVSTLYPHVRQDEFVDFAQKLFQDTDEIRSLTLLALSSQYPIKRQGETE

SEQ ID NO: 128 - DNA, 375 bp, Artificial sequence
  Synthetic
atgaccgtggccaccaccaagaccagagaatacgccgagatcgtgtacaagatcgcccagggcttcgtgctgtccaagctgagcagcaagcacgacctgcagtggtccaagtgcaagggcaaccccaagctggaaagagagtacaacgacaaaaaagagaaggtcgtcaacgaggccttcctggctatcagaagccggacagagaagcaggccttcatcgactacttcgtgtccacactgtaccctcacgtgcggcaggacgagttcgtggattttgcccagaagctgttccaggacaccgacgaaatcagatctctgaccctgctggccctgtcctctcagtaccctatcaagagacagggcgagacagagtga

SEQ ID NO: 129 - protein, 784 aa, Synechocystis sp. PCC6803
MLKQLLAKSLPTDPQKKPLSLEQHLLDTETAALVIFKGRMLDNWCRFFKVKDPDEFLLHLRVAALFHDLGKANHEFIEAVTAKGFVPQTLRHEWISALVLHLPEVRQWLGKSNLNLEVVTAAVLSHHLKASPDGDYKWDEPQKSGDKVETKLYFNHEEVDRILNKIANLLDVDSKLPELPKKWIKGDIFLENIYKDANQIGRKFTRQAKKDDSLKGLLLAVKAGLIASDSVASGIYRTQDSEAIANWVNQTLHTNSITPEEIEEKILHPRYRQVEKSINEPFQLKRFQEKAETLSSRLLLMSGCGSGKTIFAYKWMQGVLNKHQAGRAIFLYPTRGTATEGFKDYVSWCPEADASLLTGTATYELQAIAKNPTEANEGKDYQADERLYALGYWGKRFFSATVDQFLSFLTHNYKSICLLPVLADSVVVIDEIHSFSPEMFDSLVCFLKTFDVPVLCMTATLPQTRIEDLTIQLDKDKDGLGLEVFPTSDRSELAELEKAEGMERYLIAHTNEEAALDLAVKAYQDSKRVLWVVNTVDRCREKARKLECLLKTEVLTYHSRFKLADRQNRHRETVEAFALHQAQGEKKAAIAVTTQVCEMSLDLDADVLITELAPISSLVQRFGRSNRGDKNDKTEPSKIYVYKPPKDKPYKQKDDLDPAEKFINDVLGRASQKLLAEKLKEHSPPGRYSDGSAPFVTQGYWASSDEPFRKIDDFAVNAVLTEDLGEITQYLNSNPPKPIDGFIVPVPKKYKFQGFSHRPPQLPKYLEIADSKFYSSKRGFGDDA

SEQ ID NO: 130 - DNA, 2415 bp, Artificial sequence
  Synthetic
atgctgaaacagctgctggccaagagcctgcctaccgatcctcagaagaagcccctgagcctggaacagcatctgctggacacagagacagccgctctggtcatcttcaagggcagaatgctggacaactggtgccggttcttcaaagtgaaggaccccgacgagttcctgctgcacctgagagtggccgctctgtttcacgatctgggcaaagccaaccacgagttcatcgaggccgtgaccgccaagggattcgtgcctcagacactgagacacgagtggatctctgccctggtgctgcatctgcctgaagttcgacagtggctgggcaagagcaacctgaacctggaagtggttacagccgccgtgctgagccaccacctgaaagcttctcccgacggcgactacaagtgggacgagcctcagaaaagcggcgacaaggtggaaacaaagctgtacttcaaccacgaagaggtggaccggatcctgaacaagatcgccaacctgctggacgtggacagcaagctgcctgagctgcccaagaagtggatcaagggcgacatcttcctggaaaacatctacaaggacgccaaccagatcggccggaagttcaccagacaggccaagaaggacgacagcctgaagggactgctgctggctgtgaaggccggactgatcgcctctgattctgtggccagcggcatctacagaacccaggactctgaggccattgccaactgggtcaaccagacactgcacaccaacagcatcacccctgaggaaatcgaggaaaagattctgcaccctcggtacagacaggtggaaaagagcatcaacgagcccttccagctgaagcggttccaagagaaggccgagactctgagcagtcggctgctgctgatgtctggctgtggctctggcaagaccatcttcgcctataagtggatgcagggcgtgctgaacaagcaccaggccggcagagccatctttctgtaccctacaagaggcaccgccaccgagggcttcaaggactatgtgtcctggtgtcctgaggccgatgcctctctgctgactggcacagccacatacgagctgcaggctatcgccaagaatcccaccgaggccaacgagggcaaagactaccaggccgacgagagactgtacgccctcggctattggggcaagagattcttcagcgctaccgtggaccagttcctgagctttctgacccacaactacaagagcatctgtctgctgcccgtgctggccgatagcgtggtggttatcgatgagatccacagcttcagccccgagatgttcgacagcctcgtgtgcttcctgaaaaccttcgacgtgccagtgctgtgcatgaccgctacactgccccagaccagaatcgaggacctgaccatccagctcgacaaggacaaggatggcctgggcctcgaggtgttccctacctctgatagaagcgagctggccgagctggaaaaggccgaaggcatggaaagatacctgatcgcccacaccaacgaggaagctgccctggatctggccgtgaaagcctaccaggatagcaagagggtgctgtgggtcgtgaacaccgtggacagatgcagagagaaagcccggaagctggaatgcctgctgaaaaccgaggtgctgacctaccacagccggttcaaactggccgaccggcagaacagacaccgggaaaccgtggaagccttcgctctgcatcaggcccagggcgagaaaaaagccgccattgccgtgaccacacaagtgtgcgagatgtccctggacctggacgccgatgtgctgatcacagagctggcccctatcagcagcctggtgcagagattcggcagaagcaaccggggcgacaagaacgacaagaccgagcctagcaagatctacgtgtacaagcctcctaaggacaagccctacaagcagaaggatgatctggaccccgccgagaagttcatcaacgacgttctgggcagagcctctcagaagctcctggccgagaagctgaaagagcacagccctccaggcagatactccgatggatctgcccctttcgtgacccaaggctactgggcctctagcgacgagcctttcagaaagatcgacgacttcgccgtgaacgcagtgctgacagaggatctgggcgagatcacccagtacctgaacagcaaccctcctaagcctatcgacggcttcatcgtgcccgtgcctaagaagtacaagttccagggcttcagccaccggccacctcagctgcctaagtacctggaaatcgccgacagcaagttctacagcagcaagagaggctttggcgacgacgccggcagcgtcggctacccctacgatgtgcctgattacgcccctaagaaaaagcggaaagtgtga

SEQ ID NO: 131 - DNA, 36 bp, Artificial sequence; identical to SEQ ID NO: 81
  Synthetic

SEQ ID NO: 132 - DNA, 35 bp, Artificial sequence
  Synthetic
gcaccggcagcaccggcagcggcagctccggcacc

SEQ ID NO: 133 - protein, 975 aa, Synechocystis sp. PCC6803
MTTLLQTLLIRTLSEQKDYILLEYFQTILPALEEHFGNTSGLGGSFISHQKHFGTQGYDTEKAKKMAQGFAKKGDQTLAAHILNALLTTWNVMQELEFPLNDIERRLLCLGITLHDYDKHCHAQDMAAPEPDNIQEIINICLELGKRLNFDEFWADWRDYIAEISYLAQNTHGKQHTNLISSNWSNAGYPFTIKERKLDHPLRHLLTFGDVAVHLSSPHDLVSSTMGDRLRDLLNRLGIEKRFVYHHLRDTTGILSNAIHNVILRTVQKLDWKPLLFFAQGVIYFAPQDTEIPERNEIKQIVWQGISQELGKKMSAGDVGFKRDGKGLKVSPQTSELLAAADIVRILPQVISVKVNNAKSPATPKRLEKLELGDAEREKLYEVADLRCDRLAELLGLVQKEIFLLPEPFIEWVLKDLELTSVIMPEETQVQSGGVNYGWYRVAAHYVANHATWDLEEFQEFLQGFGDRLATWAEEEGYFAEHQSPTRQIFEDYLDRYLEIQGWESDHQAFIQELENYVNAKTKKSKQPICSLSSGEFPSEDQMDSVVLFKPQQYSNKNPLGGGQIKRGISKIWSLEMLLRQAFWSVPSGKFEDQQPIFIYLYPAYVYAPQVVEAIRELVYGIASVNLWDVRKHWVNNKMDLTSLKSLPWLNEEVEAGTNAQLKYTKEDLPFLATVYTTTREKTDTDAWVKPAFLALLLPYLLGVKAIATRSMVPLYRSDQDFRESIHLDGVAGFWSLLGIPTDLRVEDITPALNKLLAIYTLHLAARSSPPKARWQDLPKTVQEVMTDVLNVFALAEQGLRREKRDRPYESEVTEYWQFAELFSQGNIVMTEKLKLTKRLVEEYRRFYQVELSKKPSTHAILLPLSKALEQILSVPDDWDEEELILQGSGQLQAALDRQEVYTRPIIKDKSVAYETRQLQELEAIQIFMTTCVRDLFGEMCKGDRAILQEQRNRIKSGAEFAYRLLALEAQQNQN

SEQ ID NO: 134 - DNA, 2994 bp, Artificial sequence
  Synthetic
atggtgcccaagaaaaagcggaaggtgtacccctacgacgtgcccgattatgccggctctgtgggaatgaccacactgctgcagacactgctgatccggaccctgtccgagcagaaggactacatcctgctggagtatttccagacaatcctgccagccctggaggagcactttggaaacaccagcggactgggaggatccttcatctctcaccagaagcactttggcacacagggctacgacaccgagaaggccaagaagatggcccagggcttcgccaagaagggcgatcagacactggccgcccacatcctgaacgccctgctgaccacatggaatgtgatgcaggagctggagtttcctctgaacgatatcgagcggagactgctgtgcctgggcatcaccctgcacgactatgataagcactgtcacgcacaggacatggcagcaccagagccagataacatccaggagatcatcaatatctgcctggagctgggcaagaggctgaatttcgacgagttttgggccgactggcgcgattacatcgccgagatcagctatctggcccagaacacacacggcaagcagcacaccaatctgatcagctccaactggtccaatgccggctaccccttcacaatcaaggagcggaagctggatcaccctctgagacacctgctgacctttggcgacgtggccgtgcacctgtctagccctcacgatctggtgtcctctaccatgggcgacaggctgcgcgatctgctgaacaggctgggcatcgagaagcggttcgtgtaccaccacctgcgggacaccacaggcatcctgagcaacgccatccacaatgtgatcctgagaacagtgcagaagctggactggaagccactgctgttctttgcccagggcgtgatctatttcgccccacaggataccgagatccccgagagaaatgagatcaagcagatcgtgtggcagggcatctctcaggagctgggcaagaagatgagcgccggcgacgtgggctttaagagggatggcaagggcctgaaggtgtccccacagacatctgagctgctggcagcagcagacatcgtgcgcatcctgccccaggtcatctccgtgaaggtgaacaatgccaagtctcccgccacccctaagcggctggagaagctggagctgggcgatgccgagagagagaagctgtacgaggtggccgacctgcggtgcgatagactggccgagctgctgggcctggtgcagaaggagatcttcctgctgcctgagcccttcatcgagtgggtgctgaaggacctggagctgacatctgtgatcatgccagaggagacccaggtgcagagcggaggcgtgaactacggctggtatcgggtggccgcccactacgtggccaatcacgccacctgggacctggaggagttccaggagtttctgcagggcttcggcgatagactggccacatgggccgaggaggagggctatttcgccgagcaccagtctcctacccggcagatctttgaggactacctggatagatatctggagatccagggctgggagagcgaccaccaggcctttatccaggagctggagaactacgtgaatgccaagaccaagaagtccaagcagccaatctgttctctgagctccggcgagttccccagcgaggaccagatggattccgtggtgctgtttaagccccagcagtattccaacaagaatcctctgggaggaggacagatcaagaggggaatcagcaagatctggtccctggagatgctgctgagacaggccttctggagcgtgccctccggcaagttcgaggaccagcagcctatctttatctacctgtatcctgcctacgtgtatgccccacaggtggtggaggccatcagggagctggtgtacggcatcgccagcgtgaacctgtgggacgtgcgcaagcactgggtgaacaataagatggatctgacatctctgaagagcctgccttggctgaacgaggaggtggaggccggcacaaatgcccagctgaagtacaccaaggaggacctgccattcctggccaccgtgtataccacaaccagggagaagacagacaccgatgcctgggtgaagccagcctttctggccctgctgctgccatacctgctgggagtgaaggcaatcgcaacccgctctatggtgcccctgtatcggagcgaccaggatttcagagagtccatccacctggatggagtggcaggattttggtccctgctgggaatccctacagacctgagggtggaggatatcaccccagccctgaataagctgctggccatctataccctgcacctggcagcaaggtctagcccacctaaggcaaggtggcaggacctgcccaagacagtgcaggaagtgatgaccgatgtgctgaacgtgttcgcactggcagagcagggactgaggagggagaagagggacagaccttacgagtccgaggtgacagagtattggcagttcgccgagctgttttctcagggcaatatcgtgatgacagagaagctgaagctgaccaagcgcctggtggaggagtaccggcggttctaccaggtggagctgtctaagaagcctagcacccacgccatcctgctgccactgagcaaggccctggagcagatcctgtccgtgccagacgattgggatgaggaggagctgatcctgcagggatccggacagctgcaggccgccctggacaggcaggaggtgtacacacgccccatcatcaaggataagtctgtggcctatgagaccaggcagctgcaggagctggaggcaatccagatcttcatgacaacctgcgtgagggacctgtttggcgagatgtgcaagggcgatcgcgccatcctgcaggagcagaggaaccgcatcaagagcggcgccgagttcgcctacagactgctggccctggaggcccagcagaaccagaattaa

SEQ ID NO: 135 - protein, 329 aa, Synechocystis sp. PCC6803
MLDSLKSQFQPSFPRLASGHYVHFLMLRHSQSFPVFQTDGVLNTTRTQAGLLEKTDQLSRLVMFKRKQTTPERLAGRELLRNLGLTSADKSAKNLCEYNGEGSCKQCPDCILYGFAIGDSGSERSKVYSDSAFSLGAYEQSHRSFTFNAPFEGGTMSEAGVMRSAINELDHILPEVTFPTVESLRDATYEGFIYVLGNLLRTKRYGAQESRTGTMKNHLVGIVFADGEIFSNLHLTQALYDQMGGELNKPISELCETAATVAQDLLNKEPVRKSELIFGAHLDTLLQEVNDIYQNDAELTKLLGSLYQQTQDYATEFGALSGGKKKAKS

SEQ ID NO: 136 - DNA, 1050 bp, Artificial sequence
  Synthetic
atgctggacagcctgaagtcccagttccagccaagctttcctaggctggcatccggacactacgtgcactttctgatgctgcggcacagccagtccttccccgtgtttcagaccgacggcgtgctgaacaccacaaggacccaggcaggactgctggagaagacagatcagctgtccagactggtcatgttcaagaggaagcagaccacacctgagaggctggcaggaagggagctgctgaggaatctgggcctgacatccgccgacaagtctgccaagaacctgtgcgagtacaatggcgagggcagctgcaagcagtgtccagactgcatcctgtatggcttcgccatcggcgattctggcagcgagagaagcaaggtgtactccgattctgccttttccctgggcgcctatgagcagtctcacaggagcttcacctttaacgcccccttcgagggcggcacaatgtctgaggccggcgtgatgcgcagcgccatcaatgagctggaccacatcctgccagaggtgaccttccccacagtggagtccctgcgggatgccacctacgagggctttatctatgtgctgggcaacctgctgcggacaaagagatacggcgcccaggagtctagaaccggcacaatgaagaaccacctggtgggcatcgtgttcgccgacggcgagatctttagcaatctgcacctgacccaggccctgtatgatcagatgggcggcgagctgaacaagcctatctccgagctgtgcgagaccgcagcaacagtggcacaggacctgctgaataaggagccagtgaggaagtctgagctgatctttggcgcccacctggataccctgctgcaggaggtgaacgacatctatcagaatgatgccgagctgacaaagctgctgggcagcctgtaccagcagacccaggattatgcaacagagttcggcgccctgtccggaggcaagaagaaggccaagtctggctctgtgggctatccttacgacgtgccagactacgcccctaagaaaaagcgcaaagtgtga

SEQ ID NO: 137 - protein, 254 aa, Synechocystis sp. PCC6803
MTKIYRCKLTLHDNVFFASREMGILYETEKYFHNWALSYAFFKGTIIPHPYGLVGQNAQTPAYLDRDREQNLLHLNDSGIYVFPAQPIHWSYQINTFKAAQSAYYGRSVQFGGKGATKNYPINYGRAKELAVGSEFLTYIVSQKELDLPVWIRLGKWSSKIRVEVEAIAPDQIKTASGVYVCNHPLNPLDCPANQQILLYNRVVMPPSSLFSQSQLQGDYWQIDRNTFLPQGFHYGATTAIAQDSPQLSLLDTN

SEQ ID NO: 138 - DNA, 831 bp, Artificial sequence
  Synthetic
atggtgcccaagaaaaagcggaaggtgtacccctacgacgtgcccgattatgccggctctgtgggaatgaccaagatctatcggtgcaagctgacactgcacgacaacgtgttctttgcctctagagagatgggcatcctgtacgagacagagaagtattttcacaattgggccctgtcctacgccttctttaagggcaccatcatcccacacccctatggactggtgggacagaacgcacagacaccagcatacctggacagggatagagagcagaacctgctgcacctgaatgatagcggcatctacgtgttccctgcccagccaatccactggtcctatcagatcaatacctttaaggccgcccagtctgcctactatggcaggagcgtgcagttcggaggcaagggagccacaaagaactaccctatcaattatggaagggcaaaggagctggcagtgggatccgagtttctgacctacatcgtgtctcagaaggagctggacctgcccgtgtggatcaggctgggcaagtggagctccaagatcagggtggaggtggaggcaatcgcaccagaccagatcaagaccgccagcggcgtgtacgtgtgcaaccaccccctgaatcctctggattgtcccgccaaccagcagatcctgctgtacaatcgggtggtcatgcccccttctagcctgttctcccagtctcagctgcagggcgactattggcagatcgatcggaacacattcctgccacagggctttcactacggagcaaccacagcaatcgcacaggacagcccacagctgtccctgctggataccaattga

SEQ ID NO: 139 - protein, 262 aa, Synechocystis sp. PCC6803
MFDDRYSLYSVVIELGAAKKGFPTGILGRALHSQVLEWLKIGEPSLAEELHQSQISPFSISPLIGKRRSKLTEEGDRFFFRISLLNGSLLQPLLKGLEQQDKQIVMLDKFAFRLCHIHILPGSHSLARASSYALTTQAPTSSKITLKFHSATSFKIDRNTIQPFPLGDSVFNSLLRRWNHFAPEELYFPSVSWQIPVAAFELKTYSVQLKKSEIGSEGWVTYLFPDQEQAKIASVLSQFAFFAGVGRKTSMGMGQVSVNNHG

SEQ ID NO: 140 - DNA, 855 bp, Artificial sequence
  Synthetic
atggtgcccaagaaaaagcggaaggtgtacccctacgacgtgcccgattatgccggctctgtgggaatgtttgacgatcggtactccctgtattctgtggtcatcgagctgggagcagccaagaagggattcccaaccggcatcctgggcagagccctgcactctcaggtgctggagtggctgaagatcggagagccatccctggcagaggagctgcaccagagccagatctccccattttccatctctcccctgatcggcaagcggagaagcaagctgacagaggagggcgaccggttctttttcagaatctccctgctgaacggctctctgctgcagcccctgctgaagggcctggagcagcaggacaagcagatcgtgatgctggataagtttgccttcaggctgtgccacatccacatcctgccaggaagccactccctggcaagggccagctcctacgccctgaccacacaggcccctacctctagcaagatcacactgaagtttcactctgccaccagcttcaagatcgacaggaatacaatccagccctttcctctgggcgatagcgtgttcaactccctgctgaggcgctggaatcactttgcccctgaggagctgtatttcccatctgtgagctggcagatccctgtggccgccttcgagctgaagacctactccgtgcagctgaagaagtctgagatcggcagcgagggctgggtgacatatctgtttccagatcaggagcaggccaagatcgcctccgtgctgtctcagttcgcctttttcgcaggagtgggaaggaagaccagcatgggcatgggccaggtgtccgtgaacaatcacggctga

SEQ ID NO: 141 - protein, 146 aa, Synechocystis sp. PCC6803
MTEKLKLTKRLVEEYRRFYQVELSKKPSTHAILLPLSKALEQILSVPDDWDEEELILQGSGQLQAALDRQEVYTRPIIKDKSVAYETRQLQELEAIQIFMTTCVRDLFGEMCKGDRAILQEQRNRIKSGAEFAYRLLALEAQQNQN

SEQ ID NO: 142 - DNA, 441 bp, Artificial sequence
  Synthetic
atgacagagaagctgaagctgaccaagcgcctggtggaggagtaccggcggttctaccaggtggagctgtctaagaagcctagcacccacgccatcctgctgccactgagcaaggccctggagcagatcctgtccgtgccagacgattgggatgaggaggagctgatcctgcagggatccggacagctgcaggccgccctggacaggcaggaggtgtacacacgccccatcatcaaggataagtctgtggcctatgagaccaggcagctgcaggagctggaggcaatccagatcttcatgacaacctgcgtgagggacctgtttggcgagatgtgcaagggcgatcgcgccatcctgcaggagcagaggaaccgcatcaagagcggcgccgagttcgcctacagactgctggccctggaggcccagcagaaccagaattaa

SEQ ID NO: 143 - protein, 722 aa, Synechocystis sp. PCC6803
MSLPKVGAMKVQVKPLYAKLNEGLGQCPLGCQSVCTVREQAPEMQPPDGCSCPLYDHQAQTYALTTAGDTDIIFNRVATGGGKSLGATLPALLPKVHPDFRVMGLYPTIELVEDQYRQQQQYHQMFGLNAEKRVDRLYGAELARRISEKDSNRFQELLKSIEQKPVIQTNPDIFHYITHFRYRDNARSQSELPMVMAKFPDLWVFDEFHIFGEHQMAAALNSLLLIRHATQSKRKFLFTSATPKTSFVESLKNSGFKIAEIQGEYSDRPQQGYRQILQNINLEFVHLKDTDTESWLLAQSQNLRDLLHQEKNGRGLIIVNSVAAAGHLCRNLSKILPDVEVREISGRCDRQERQQTQTYLQKSEKSVLVVATSAVDVGVDFKIHVLICESSDSATVIQRLGRLGRHGGFQQYQAYVLIPSQTQWVIASLKEELEEDSSIDRLELSTVIETAFNAPQEFKQYQKIWGELQAQGMLYQMVYGGSRSESKDNLAVTQDLRDRMIESLEKIYQKQINGFGRWWGLSNDAVGKATQEELLRFRGGSSLQSGIWDGDRLYSYDLLKVLTYATVEVIDREIFLEAAQKLNHGIEEFPEQYLTGYFKITKWLDQRLNFSLFSTHSHLASCQLMLLDRLQLKGHIQPELSRCLGRRKYLAYLVPINRNNPSSHWDISRKLRLSPLFGLYRLTDSGGNAYGCAFNHDALLLEALKWRLGDFCKQATSQSLIF

SEQ ID NO: 144 - DNA, 2229 bp, Artificial sequence
  Synthetic
atgagcctgccaaaagtgggcgccatgaaggtgcaggtgaagcccctgtacgccaagctgaacgagggactgggacagtgcccactgggatgtcagagcgtgtgcacagtgcgggagcaggcacctgagatgcagccacctgacggatgcagctgtccactgtacgatcaccaggcccagacatatgccctgaccacagccggcgacaccgatatcatctttaatagagtggcaacaggaggaggcaagagcctgggagccaccctgccagccctgctgccaaaggtgcaccctgacttcagagtgatgggcctgtaccctaccatcgagctggtggaggaccagtaccgccagcagcagcagtatcaccagatgtttggcctgaacgccgagaagagggtggataggctgtatggagcagagctggcaaggagaatctctgagaaggacagcaatcggttccaggagctgctgaagtccatcgagcagaagccagtgatccagacaaaccccgacatctttcactacatcacccacttccggtatagagataatgccagatctcagagcgagctgcccatggtcatggccaagtttcctgacctgtgggtgttcgatgagtttcacatcttcggagagcaccagatggcagccgccctgaactccctgctgctgatcaggcacgccacccagtctaagcgcaagttcctgtttaccagcgccacacccaagacctcctttgtggagtctctgaagaatagcggcttcaagatcgccgagatccagggcgagtactctgatcggcctcagcagggctatagacagatcctgcagaacatcaatctggagttcgtgcacctgaaggacaccgatacagagtcctggctgctggcccagtctcagaacctgcgggacctgctgcaccaggagaagaacggcagaggcctgatcatcgtgaattctgtggcagcagcaggacacctgtgcaggaatctgagcaagatcctgccagacgtggaggtgcgcgagatctccggccggtgcgatagacaggagaggcagcagacccagacatacctgcagaagagcgagaagtccgtgctggtggtggccacaagcgccgtggacgtgggcgtggatttcaagatccacgtgctgatctgtgagagctccgattccgccaccgtgatccagcgcctgggccggctgggcagacacggaggctttcagcagtaccaggcctatgtgctgatcccatcccagacccagtgggtcatcgcctctctgaaggaggagctggaggaggactctagcatcgataggctggagctgagcacagtgatcgagaccgcctttaacgccccccaggagttcaagcagtaccagaagatctggggagagctgcaggcacagggaatgctgtaccagatggtgtatggcggctccaggtctgagagcaaggataatctggccgtgacacaggacctgagggatcgcatgatcgagtccctggagaagatctatcagaagcagatcaacggctttggccgctggtggggcctgtctaatgacgcagtgggcaaggcaacccaggaggagctgctgcggttcagaggcggctcctctctgcagagcggcatctgggacggcgatcggctgtactcctatgatctgctgaaggtgctgacatacgccaccgtggaagtgatcgacagagagatcttcctggaggccgcccagaagctgaaccacggcatcgaggagtttcccgagcagtacctgacaggctatttcaagatcaccaagtggctggatcagaggctgaatttttctctgttcagcacccactcccacctggcctcttgtcagctgatgctgctggaccggctgcagctgaagggccacatccagcctgagctgagcagatgcctgggcaggcgcaagtacctggcctatctggtgcctatcaacaggaacaatccaagctcccactgggacatcagcaggaagctgcgcctgtccccactgtttggactgtacaggctgacagactccggaggaaacgcctatggctgtgccttcaatcacgatgcactgctgctggaggccctgaagtggaggctgggcgacttttgcaagcaggccacctcccagtctctgatcttcggcagcgtcggctacccctacgatgtgcctgattacgcccctaagaaaaagcggaaagtgtga

SEQ ID NO: 145 - DNA, 37 bp, Artificial sequence; identical to SEQ ID NO: 82
  Synthetic

SEQ ID NO: 146 - DNA, 35 bp, Artificial sequence
  Synthetic
cgtgaccgccgccgggatcactctcggcatggacg

SEQ ID NO: 147 - protein, 283 aa, Bacillus halodurans
MTILDHKIDFAVILSVTKANPNGDPLNGNRPRQNYDGHGEISDVAIKRKIRNRLLDMEEPIFVQSDDRKADSFKSLRDRADSNPELAKMLKAKNASVDEFAKIACQEWMDVRSFGQVFAFKGSNLSVGVRGPVSIHTATSIDPIDIVSTQITKSVNSVTGDKRSSDTMGMKHRVDFGVYVFKGSINTQLAEKTGFTNEDAEKIKRALITLFENDSSSARPDGSMEVHKVYWWEHSSKLGQYSSAKVHRSLKIESKTDTPKSFDDYAVELYELDGLGVEVIDGQ

SEQ ID NO: 148 - DNA, 912 bp, Artificial sequence
  Synthetic
atggtgcctaagaagaagagaaaggtgtacccatacgatgttccagattacgctggcagcggcaccatcctggaccacaagatcgacttcgccgtgatcctgagcgtgaccaaggccaatcctaacggcgaccctctgaacggcaacagacccagacagaactacgatggccacggcgagatcagcgacgtggccattaagcggaagatccggaaccggctgctggacatggaagaacccatcttcgtgcagagcgacgacagaaaggccgacagcttcaagagcctgagagacagagccgacagcaaccctgagctggccaagatgctgaaggccaagaatgccagcgtggacgagttcgccaagatcgcctgtcaagagtggatggacgtgcggagctttggccaggtgttcgccttcaagggcagcaatctgagcgtgggcgttagaggccctgtgtctattcacaccgccaccagcatcgaccccatcgacatcgtgtctacccagatcaccaagagcgtgaacagcgtgaccggcgacaagagaagcagcgacaccatgggcatgaagcacagagtggacttcggcgtgtacgtgttcaagggctccatcaacacccagctggccgagaaaaccggcttcaccaatgaggacgccgagaagatcaagcgggccctgatcaccctgttcgagaacgatagcagcagcgccagacctgacggcagcatggaagtgcacaaagtgtattggtgggagcacagcagcaagctgggccagtactctagcgccaaggtgcacagaagcctgaagatcgagagcaagaccgacacacccaagagcttcgacgactacgccgtggaactgtacgagctggatggcctgggcgtcgaagtgatcgatggacaataa

SEQ ID NO: 149 - protein, 236 aa, Bacillus halodurans
MRNEVQFELFGDYALFTDPLTKIGGEKLSYSVPTYQALKGIAESIYWKPTIVFVIDELRVMKPIQMESKGVRPIEYGGGNTLAHYTYLKDVHYQVKAHFEFNLHRPDLAFDRNEGKHYSILQRSLKAGGRRDIFLGARECQGYVAPCEFGSGDGFYDGQGKYHLGTMVHGFNYPDETGQHQLDVRLWSAVMENGYIQFPRPEDCPIVRPVKEMEPKIFNPDNVQSAEQLLHDLGGE

SEQ ID NO: 150 - DNA, 762 bp, Artificial sequence
  Synthetic
atgagaaacgaggtgcagttcgagctgttcggcgactacgccctgttcaccgatcctctgacaaagatcggcggcgagaagctgagctacagcgtgccaacatatcaggccctgaagggaatcgccgagagcatctactggaagcccaccatcgtgttcgtgatcgacgagctgagagtgatgaagcccatccagatggaaagcaaaggcgtgcggcccatcgagtacggcggaggaaatactctggcccactacacctacctgaaggacgtgcactaccaagtgaaggcccacttcgagttcaacctgcacagacccgacctggccttcgacagaaatgagggcaagcactacagcatcctgcagcggagtctgaaggctggcggcagaagagacatcttcctgggcgctagagaatgccagggctatgtggccccttgcgagtttggaagcggcgacggcttttatgacggccagggcaagtatcacctgggcacaatggtgcacggcttcaactaccccgatgagacaggacagcaccagctggatgttcggctttggagcgccgtgatggaaaacggctacattcagttccccagacctgaggactgccccattgtgcggcctgtgaaagagatggaacccaagatcttcaaccccgacaacgtgcagtctgccgagcagctcctgcatgatcttggcggagaatacccatacgatgttccagattacgctgtgcctaagaagaagagaaaggtgtaa

SEQ ID NO: 151 - protein, 627 aa, Bacillus halodurans
MSWLLHLYETYEANLDQVGKTVKKGEDREYTLLPISHTTQNAHIEVTLDEDGDFLRAKALTKESTLIPCTEEAASRSGSKVAPYPLHDKLSYVAGDFVKYGGKIKNQDDAPFDTYIKNLGEWANSPYATEKVKCIYTYLKKGRLIEDLVDAGVLKLDENQQLIEKWEKRYEELLGEKPAIFSSGATDQASAFVRFNVFHPESIDDVWKDKEMFDSFISFYNDKLGEEDICFVTGNRLPSTERHANKIRHAADKAKLISANDNSGFTFRGRFKTSREAVGISYEVSQKAHNALKWLIHRQSKSIDDRVFLVWSNDNSLVPNPDEDAVDIMKHANRELERDPDTGQIFAGEVKKAIGGYRSDLNYQPEVHILVLDSATTGRMAVLYYRSLNKELYLNRLEAWHDSCAWEHRYRRDEKEFISFYGAPATKDIAFAAYGPRASEKVIKDLMERMLPCIVDGRRVPKDIVRSAFQRASNPVSMERWEWEKTLSITCALIRKMHIEQKEEWGVPLDKSSTDRSYLFGRLLAVADVLERGALGKDETRATNAIRYMNSYSKNPGRTWKTIQESLQPYQAKLGTKATYLSKLVDEIGDQFEPGDFNNNPLTEQYLLGFYSQRRELYKKKEEETNQ

SEQ ID NO: 152 - DNA, 2004 bp, Artificial sequence
  Synthetic
atggttcccaagaagaagcggaaagtctggtcccatcctcagttcgagaaaggcggaggatctggcggtggttctggtggatctgcttggagccatccacaatttgaaaaaggcagcggcatgagctggctgctgcacctgtacgagacatacgaggccaacctggaccaagtgggcaagaccgtgaagaagggcgaagatagagagtacaccctgctgcctatcagccacaccacacagaacgcccacatcgaagtgaccctggacgaggacggcgacttcctgagagccaaggctctgaccaaagagagcacactgatcccttgcaccgaggaagccgccagcagatctggctctaaggtggccccttatcctctgcacgacaagctgtcttacgtggccggcgatttcgtgaagtacggcggcaagatcaagaaccaggacgacgcccctttcgacacctacatcaagaatctcggcgagtgggccaactctccctacgccacagagaaagtgaagtgcatctacacctacctgaagaaaggccggctgatcgaggacctggtggatgctggtgtcctgaagctggacgagaaccagcagctgattgagaagtgggagaagcgctacgaggaactgctgggagagaagcccgccatctttagctctggcgccacagatcaggccagcgccttcgtgcggttcaatgtgtttcaccccgagagcatcgacgacgtgtggaaggacaaagagatgttcgacagcttcatcagcttctacaacgataagctgggcgaagaggacatctgcttcgtgaccggcaacagactgcccagcacagagagacacgccaacaagattagacacgccgccgacaaggccaagctgatctccgccaatgacaacagcggcttcaccttccggggcagattcaagaccagcagagaagccgtgggcatcagctacgaggtgtcccagaaagcccacaacgccctgaagtggctgatccacagacagagcaagtctatcgacgaccgggtgttcctcgtgtggtccaacgacaatagcctggtgcctaatcctgacgaggatgccgtggacatcatgaagcacgccaatagagagctggaacgggaccctgataccggccagatttttgccggcgaagtgaagaaagccatcggcggctacagaagcgacctgaactatcagcccgaggtgcacatcctggtgctggattctgccaccaccggaagaatggctgtgctgtactaccgcagcctgaacaaagagctgtacctgaaccggctggaagcctggcacgattcttgtgcctgggagcacagataccggcgggacgagaaagagttcatctccttctacggcgctcccgccaccaaggatattgcctttgccgcctatggacccagagccagcgaaaaagtgatcaaggatctgatggaacggatgctgccctgcatcgtggacggcagaagagtgcctaaggacattgtgcggagcgccttccagagggccagcaatcctgtgtccatggaaagatgggagtgggaaaagaccctgagcatcacatgcgccctgatccggaagatgcacatcgagcagaaagaggaatggggcgtcccactggacaagagcagcaccgatagaagctacctgttcggcagactgctggccgtggctgacgttttggaaagaggcgccctgggcaaagacgagacaagagccacaaacgccatccggtacatgaacagctacagcaagaaccccggcagaacctggaaaaccatccaagagagcctgcagccttaccaggccaaactgggcaccaaggccacatacctgagcaagctggtggacgagatcggggaccagtttgagcccggcgactttaacaacaaccctctgaccgagcagtacctgctgggcttctacagccagcggcgggaactgtacaagaagaaagaagaggaaacgaaccagtaa

SEQ ID NO: 153 - protein, 121 aa, Bacillus halodurans
MPLDKSSTDRSYLFGRLLAVADVLERGALGKDETRATNAIRYMNSYSKNPGRTWKTIQESLQPYQAKLGTKATYLSKLVDEIGDQFEPGDFNNNPLTEQYLLGFYSQRRELYKKKEEETNQ

SEQ ID NO: 154 - DNA, 366 bp, Artificial sequence
  Synthetic
atgccactggacaagagcagcaccgatagaagctacctgttcggcagactgctggccgtggctgacgttttggaaagaggcgccctgggcaaagacgagacaagagccacaaacgccatccggtacatgaacagctacagcaagaaccccggcagaacctggaaaaccatccaagagagcctgcagccttaccaggccaaactgggcaccaaggccacatacctgagcaagctggtggacgagatcggggaccagtttgagcccggcgactttaacaacaaccctctgaccgagcagtacctgctgggcttctacagccagcggcgggaactgtacaagaagaaagaagaggaaacgaaccagtaa

SEQ ID NO: 155 - protein, 800 aa, Bacillus halodurans
MYIAHIREVDKVIQTLKEHLCGVQCLAETFGAKLRLQHVAGLAGLLHDLGKYTNEFKDYIYKAVFEPELAEKKRGQVDHSTAGGRLLYQMLHDRENSFHEKLLAEVVGNAIISHHSNLQDYISPTIESNFLTRVLEKELPEYESAVERFFQEVMTEAELARYVAKAVDEIKQFTDNSPTQSFFLTKYIFSCLIDADRTNTRMFDEQAREEEPTQPQQLFEHYHQQLLNHLASLKESDSAQKPINVLRSAMSEQCESFAMRPSGIYTLSIPTGGGKTLASLRYALKHAQEYNKQRIIYIVPFTTIIEQNAQEVRNILGDDENILEHHSNVVEDSENGDEQEDGVITKKERLRLARDNWDRPIIFTTLVQFLNVFYAKGNRNTRRLHNLSHSVLIFDEVQKVPTKCVSLFNEALNFLKEFAHCSILLCTATQPTLENVKHSLLKDRDGEIVQNLTEVSEAFKRVEILDKTDQPMTNERLAEWVRDEAPSWGSTLIILNTKKVVKDLYEKLEGGPLPVFHLSTSMCAAHRKDQLDEIRALLKEGTPFICVTTQLIEAGVDVSFKCVIRSLAGLDSIAQAAGRCNRHGEEQLQYVYVIDHAEETLSKLKEIEVGQEIAGNVLARFKKKAEKYEGNLLSQAAMREYFRYYYSKMDANLNYFVKEVDKDMTKLLMSHAVENSYVTYYQKNTGTHFPLLLNGSYKTAADHFRVIDQNTTSAIVPYGEGQDIIAQLNSGEWVDDLSKVLKKAQQYTVNLYSQEIDQLKKEGAIVMHLDGMVYELKESWYSHQYGVDFKGEGGMDFMSF

SEQ ID NO: 156 - DNA, 2520 bp, Artificial sequence
  Synthetic
atgtatatcgcccacatccgcgaggtggacaaagtgatccagacactgaaagaacacctgtgcggcgtgcagtgcctggccgaaacatttggagccaagctgagactgcagcacgtggcaggactggctggactgctgcatgatctgggcaagtacaccaacgagttcaaggactacatctacaaggccgtgttcgagcccgagctggccgagaaaaagagaggccaggtcgaccactctaccgctggtggcagactgctgtaccagatgctgcacgacagagagaacagcttccacgagaagctgctggccgaagtcgtgggcaatgccatcatcagccaccacagcaacctgcaggattacatcagccctacaatcgagagcaacttcctgaccagagtgctggaaaaagagctgcccgagtacgagagcgccgtggaacggttcttccaagaagtgatgaccgaggccgaactggccagatacgtggccaaagccgtggacgagatcaagcagttcaccgacaacagccctactcagtcattctttctgaccaagtacatcttcagctgcctgatcgacgccgaccggaccaacaccagaatgttcgatgagcaggccagagaggaagaacccacacagcctcagcagctgttcgagcactatcaccagcaactgctgaaccacctggccagcctgaaagagagcgacagcgcccagaaacctatcaacgtgctgagaagcgccatgagcgagcagtgcgagagctttgccatgaggcctagcggcatctacaccctgtctatcccaacaggcggcggaaagaccctggcttctctgagatatgccctgaagcacgcccaagagtacaacaagcagcggatcatctacatcgtgcccttcaccaccatcatcgagcagaacgctcaagaagtgcggaacatcctgggcgacgacgagaatatcctggaacaccactccaacgtggtggaagatagcgagaacggcgacgagcaagaggacggcgtgatcaccaagaaagagagactgagactggcccgggacaactgggacagacccatcatctttacaaccctggtgcagttcctgaacgtgttctacgccaagggcaacagaaacaccagacggctgcacaacctgagccacagcgtgctgatcttcgacgaggtgcagaaagtgcccaccaaatgcgtgtccctgttcaacgaggccctgaactttctgaaagagttcgcccactgcagcatcctgctgtgcactgccactcagcccacactggaaaacgtgaagcacagcctgctgaaggaccgcgacggcgagattgtgcagaacctgaccgaagtgtccgaggccttcaagagagtggaaatcctggacaagaccgaccagcctatgaccaacgaaagactggccgagtgggtccgagatgaggctccttcttggggctctaccctgatcatcctgaataccaagaaggtggtcaaggacctgtatgagaagctggaaggcggccctctgcctgtgtttcacctgagcacctctatgtgcgccgctcacagaaaggaccagctggatgagatcagagccctgctgaaagagggcacccctttcatctgcgtgaccacacagctgattgaggccggcgtggacgtgtcctttaagtgcgtgatcagaagcctggccggcctggattctattgcccaggctgccggaagatgcaacagacacggcgaagaacagctccagtacgtgtacgtgatcgaccacgccgaggaaaccctgagcaagctgaaagaaatcgaagtgggccaagagatcgccggcaatgtgctggcccggttcaagaagaaggccgagaagtacgagggcaacctgctgtctcaggccgccatgagagagtacttccggtactactacagcaagatggacgccaacctgaactacttcgtgaaagaagtcgacaaggacatgaccaagctgctgatgagccacgccgtcgagaactcctacgtgacctactaccagaagaacaccggcacacacttccctctgctgctgaacggcagctacaagacagccgccgaccacttcagagtgattgaccagaataccaccagcgccatcgtgccttatggcgaaggccaggacatcattgcccagctgaatagcggcgagtgggttgacgatctgagcaaggtgctgaagaaagcccagcagtacaccgtgaacctgtactcccaagagattgatcagctgaaaaaagagggcgccattgtgatgcacctggacggcatggtgtacgaactcaaagagagctggtactcccaccagtatggcgtggacttcaaaggcgaaggcggcatggacttcatgagcttcggcagcggctggtcccatcctcagttcgagaaaggcggaggatctggcggtggttctggtggatctgcttggagccatccacaatttgaaaaagtgcctaagaagaagagaaaggtgtaa

SEQ ID NO: 157 - DNA, 31 bp, Artificial sequence; identical to SEQ ID NO: 79
  Synthetic

SEQ ID NO: 158 - DNA, 36 bp, Artificial sequence
  Synthetic
gagggcgacaccctggtgaaccgcatcgagctgaag

SEQ ID NO: 159 - protein, 227 aa, Desulfovibrio vulgaris
MTHGAVKTYGIRLRVWGDYACFTRPEMKVERVSYDVMPPSAARGILEAIHWKPAIRWIVDRIHVLRPIVFDNVRRNEVSSKIPKPNPATAMRDRKPLYFLVDDGSNRQQRAATLLRNVDYVIEAHFELTDKAGAEDNAGKHLDIFRRRARAGQSFQQPCLGCREFPASFELLEGDVPLSCYAGEKRDLGYMLLDIDFERDMTPLFFKAVMEDGVITPPSRTSPEVRA

SEQ ID NO: 160 - DNA, 744 bp, Artificial sequence
  Synthetic
atgacacacggggccgtgaaaacctacggcatcagactgagagtgtggggcgactacgcctgcttcaccagacctgagatgaaggtggaacgggtgtcctacgacgtgatgcctccatctgccgccagaggaatcctggaagccatccactggaagcccgccatcagatggatcgtggacagaatccacgtgctgcggcccatcgtgttcgacaacgtgcggagaaatgaggtgtccagcaagatccccaagcctaatcctgccaccgccatgagagacagaaagcccctgtacttcctggtggacgacggcagcaacagacagcagagagctgccacactgctgcggaacgtggactatgtgatcgaggcccacttcgagctgaccgataaggctggcgccgaggataatgccggcaagcacctggacatcttccggcggagagctagagccggccagtcttttcagcagccttgcctgggctgcagagagttccctgcctctttcgaactgctggaaggcgacgtgcccctgtcttgttatgccggcgagaagcgcgatctgggctacatgctgctggacatcgacttcgagcgggacatgacccctctgttcttcaaggccgtgatggaagatggcgtgatcacccctcctagccggacatctcctgaagttcgagctggcagcgtcggctacccctacgatgtgcctgattacgcccctaagaaaaagcggaaagtgtga

SEQ ID NO: 161 - protein, 612 aa, Desulfovibrio vulgaris
MILQALHGYYQRMSADPDAGMPPYGTSMENISFALVLDAKGTLRGIEDLREQEGKKLRPRKMLVPIAEKKGNGIKPNFLWENTSYILGVDAKGKQERTDKCHAAFIAHIKAYCDTADQDLAAVLQFLEHGEKDLSAFPVSEEVIGSNIVFRIEGEPGFVHERPAARQAWANCLNRREQGLCGQCLITGERQKPIAQLHPSIKGGRDGVRGAQAVASIVSFNNTAFESYGKEQSINAPVSQEAAFSYVTALNYLLNPSNRQKVTIADATVVFWAERSSPAEDIFAGMFDPPSTTAKPESSNGTPPEDSEEGSQPDTARDDPHAAARMHDLLVAIRSGKRATDIMPDMDESVRFHVLGLSPNAARLSVRFWEVDTVGHMLDKVGRHYRELEIIPQFNNEQEFPSLSTLLRQTAVLNKTENISPVLAGGLFRAMLTGGPYPQSLLPAVLGRIRAEHARPEDKSRYRLEVVTYYRAALIKAYLIRNRKLEVPVSLDPARTDRPYLLGRLFAVLEKAQEDAVPGANATIKDRYLASASANPGQVFHMLLKNASNHTAKLRKDPERKGSAIHYEIMMQEIIDNISDFPVTMSSDEQGLFMIGYYHQRKALFTKKNKEN

SEQ ID NO: 162 - DNA, 1902 bp, Artificial sequence
  Synthetic
atggtgcccaagaaaaagcggaaggtgtacccctacgacgtgcccgattatgccggctctgtgggaattctgcaggccctgcacggctactaccagagaatgagcgccgatcctgacgccggcatgcctccttatggcaccagcatggaaaacatcagcttcgccctggtgctggacgccaagggaacactgagaggcatcgaggacctgagagagcaagagggcaagaagctgcggcccagaaagatgctggttcctatcgccgagaagaaaggcaacggaatcaagcccaacttcctgtgggagaacaccagctacatcctcggcgtggacgctaagggcaagcaagagcggaccgataagtgccacgccgcctttatcgcccacatcaaggcctactgcgacaccgccgatcaggatctggctgccgtgctgcagtttctggaacacggcgagaaggatctgagcgcctttcctgtgtccgaggaagtgatcggcagcaacatcgtgttccggatcgagggcgagcctggctttgtgcacgaaagacctgctgccagacaggcctgggccaactgcctgaatagaagagaacagggcctgtgcggccagtgcctgattacaggcgagagacagaagcctatcgctcagctgcaccccagcatcaaaggcggaagagatggcgttagaggcgcccaggctgtggccagcatcgtgtcctttaacaacaccgccttcgagagctacggcaaagagcagagcatcaacgcccctgtgtctcaagaggccgccttcagctatgtgacagccctgaactacctgctgaacccctccaaccggcagaaagtgacaatcgccgatgccaccgtggtgttttgggccgagagatctagccctgccgaggatatctttgccggcatgttcgaccctccaagcaccacagccaagccagagtccagcaatggcacccctcctgaggatagcgaggaaggctctcagcctgacaccgccagagatgatcctcatgccgccgctagaatgcacgatctgctggtggccatcagatctggcaaacgggccaccgacatcatgcccgacatggatgagagcgtgcggttccatgtgctgggactgtctccaaatgccgccagactgtccgtgcgcttctgggaagttgataccgtgggccacatgctggacaaagtgggcagacactacagagagctggaaatcatccctcagttcaacaacgagcaagagttccccagcctgagcaccctgctgagacagacagccgtgctgaacaagaccgagaacatcagcccagtgctggctggcggactgttcagagctatgcttaccggcggaccctatccacagtctctgctgcctgctgtgctgggcagaattagagccgaacacgccagaccagaggacaagagccggtacagactggaagtggtcacctactaccgggctgccctgattaaggcctacctgatcagaaaccggaagctggaagtgcccgtgtctctggatcctgccagaaccgacagaccttacctgctggggagactgttcgccgtgctggaaaaggcccaagaggatgctgtgcctggcgccaatgccaccatcaaggatagatacctggccagcgcctccgccaatcctggacaggttttccatatgctgctgaagaacgccagcaaccacaccgccaagctgagaaaggaccccgagagaaagggcagcgccatccactacgagatcatgatgcaagagatcatcgacaacatcagcgacttccccgtgaccatgagcagcgacgagcaggggctgttcatgatcggctactatcaccagcggaaagccctgttcaccaagaagaacaaagagaactag

SEQ ID NO: 163 - protein, 290 aa, Desulfovibrio vulgaris
MTAIANRYEFVLLFDVENGNPNGDPDAGNMPRIDPETGHGLVTDVCLKRKIRNHVALTKEGAERFNIYIQEKAILNETHERAYTACDLKPEPKKLPKKVEDAKRVTDWMCTNFYDIRTFGAVMTTEVNCGQVRGPVQMAFARSVEPVVPQEVSITRMAVTTKAEAEKQQGDNRTMGRKHIVPYGLYVAHGFISAPLAEKTGFSDEDLTLFWDALVNMFEHDRSAARGLMSSRKLIVFKHQNRLGNAPAHKLFDLVKVSRAEGSSGPARSFADYAVTVGQAPEGVEVKEML

SEQ ID NO: 164 - DNA, 933 bp, Artificial sequence
  Synthetic
atgaccgccattgccaacagatacgagttcgtgctgctgttcgacgtggaaaacggcaaccccaacggcgatcctgacgccggcaatatgcccagaatcgaccctgagacaggccacggcctggtcacagatgtgtgcctgaagcggaagatccggaaccacgtggccctgacaaaagagggcgccgagcggttcaacatctacatccaagagaaggccatcctgaacgagacacacgagagagcctacaccgcctgcgatctgaagcccgagcctaagaaactgcccaagaaggtcgaggacgccaagcgcgtgaccgattggatgtgcaccaacttctacgacatccggaccttcggcgccgtgatgaccaccgaagtgaattgtggacaagtgcggggacccgtgcagatggcctttgccagatctgtggaacccgtggtgccccaagaggtgtccatcacaagaatggccgtgaccacaaaggccgaggccgaaaaacagcagggcgacaacagaaccatgggcagaaagcacatcgtgccctacggcctgtatgtggcccacggctttatttctgcccctctggccgagaaaaccggcttctccgatgaggatctgaccctgttctgggacgccctggtcaacatgttcgagcacgatagatctgccgccagaggcctgatgagcagcagaaagctgatcgtgttcaagcaccagaaccggctgggcaatgcccctgctcacaagctgttcgatctggtcaaggtgtccagagccgagggcagttctggacctgccagaagctttgccgattacgccgtgacagttggacaggcccctgaaggcgtggaagtgaaagagatgctgggctctgtgggctatccttacgacgtgccagactacgcccctaagaaaaagcgcaaagtgtga

SEQ ID NO: 165 - protein, 124 aa, Desulfovibrio vulgaris
MSLDPARTDRPYLLGRLFAVLEKAQEDAVPGANATIKDRYLASASANPGQVFHMLLKNASNHTAKLRKDPERKGSAIHYEIMMQEIIDNISDFPVTMSSDEQGLFMIGYYHQRKALFTKKNKEN

SEQ ID NO: 166 - DNA, 375 bp, Artificial sequence
  Synthetic
atgtctctggatcctgccagaaccgacagaccttacctgctggggagactgttcgccgtgctggaaaaggcccaagaggatgctgtgcctggcgccaatgccaccatcaaggatagatacctggccagcgcctccgccaatcctggacaggttttccatatgctgctgaagaacgccagcaaccacaccgccaagctgagaaaggaccccgagagaaagggcagcgccatccactacgagatcatgatgcaagagatcatcgacaacatcagcgacttccccgtgaccatgagcagcgacgagcaggggctgttcatgatcggctactatcaccagcggaaagccctgttcaccaagaagaacaaagagaactaa

SEQ ID NO: 167 - protein, 745 aa, Desulfovibrio vulgaris
MADGGDEHASGHDNTLKNARYYAHSTPNPDKSDWQGLDAHLENVANLAATFAEAFGAREWGKAAGLLHDAGKATAQFTQRLEGRPVRVNHSICGARLAQEQGSTCGLLLSYAIAGHHGGLPDGGLQDGQLHHRLKHERLPADVSPPSVDIRPDVLKPPFTCRPEHPGFSLSFFTRMLFSCLTDADFLDTEAFCTPEKASARNGRSALGLVALRDALNTHLDTVERKALPSRVNDIRKTVLHDCRARASETPGLFSLTVPTGGGKTLSSMAFALDHAVTHGLRRVIYAIPFTSIIEQNAKVFSDVFGQDNVLEHHCNYRSKDEPEEQGYDKWRGLAAENWDAPVVVTTNVQFFESLFSNRPSRCRKLHNIARSVIVLDEAQAIPTEYLEPCLYALKELVGQYGCTVVLCTATQPAVDDASLPERVRLHHVREIIADPQRLYTDLKRTEVTLAGRLTDAALAARLDGHGQVLCIVGTKPQAQAVFSLLQEREGAFHLSTNMYPEHRRRVLGTIRQRLADRLPCRVVSTSLIEAGVDVDFPVVYRAMAGLDSIAQAAGRCNREGRLPEPGQVVVYEPEKPARMPWMQRCASRAQETLRTLPEADPLGLEAIRRYFGLIYDVQELDRKDIFKRLRGQVDRDMVFKFREIANDFRFIDDEGTALVIPTGPEVEDLVRRLRGCEFPRPVLRKLQQYSVTVRHRELEKLRSAGAVEMIGDAYPVLRNLAAYSEDMGLCVDSVEVWQPEGLVS

SEQ ID NO: 168 - DNA, 2319 bp, Artificial sequence
  Synthetic
atggctgatggcggagatgaacacgccagcggccacgacaacaccctgaagaacgccagatattacgcccacagcacccctaatcctgacaagagcgattggcagggcctcgacgcccacctggaaaatgtggctaatctggccgccaccttcgccgaagcctttggagctagagagtggggaaaagccgccggactgctgcacgatgccggaaaagctacagcccagttcacccagagactggaaggcagacccgtcagagtgaaccactctatctgtggcgccagactggcccaagagcagggctctacttgtggcctgctgctgagctatgccattgccggacatcatggcggactgccagatggtggactgcaggatggacagctgcaccacagactgaagcacgagagactgcccgccgatgtgtctcctcctagcgtggacatcagacccgacgtgctgaagcctcctttcacctgtcggcctgagcaccctggcttcagcctgagcttcttcacccggatgctgttcagctgcctgaccgacgccgatttcctggataccgaggccttctgcacccctgagaaagcctctgccagaaatgggagatctgccctgggactcgtggccctgagagatgccctgaacacacacctggacaccgtggaaagaaaagccctgcctagccgcgtgaacgacatcagaaagaccgtgctgcatgactgcagagccagagcctctgaaacccctggcctgttctctctgacagtgcctacaggcggcggaaagaccctgagcagcatggcctttgctctggaccacgccgtgacacacggactgagaagagtgatctacgctatccccttcaccagcatcatcgagcagaacgccaaggtgttcagcgacgtgttcggccaggacaacgtgctggaacaccactgcaactacagaagcaaggacgagcccgaggaacagggctacgataagtggcgaggactggccgctgagaactgggatgctcctgtggtcgtgaccaccaacgtgcagttcttcgagagcctgttcagcaacagacccagccggtgccggaagctgcacaatatcgccagatccgtgatcgtgctggacgaggcccaggccattcctaccgagtacctggaaccttgcctgtacgccctgaaagaactcgtgggccagtacggctgtaccgtggtgctgtgtacagccacacagcccgctgtggatgatgcctctctgcctgagagagtgcggctgcatcacgtgcgcgagatcattgccgatcctcagagactgtacaccgacctgaagcggaccgaagtgacactggctggcagactgacagatgccgctctggctgctagactggatggacatggccaggtgctgtgcatcgtgggcacaaaacctcaggcacaggccgtgttcagcctgctgcaagaaagagagggcgccttccacctgtccaccaacatgtaccccgaacacaggcggagagtgctgggcaccattagacagaggctggccgatagactgccctgcagagtggtgtctaccagcctgattgaagccggcgtggacgtggacttccccgtggtgtatagagctatggccggcctggattctatcgcccaggctgccggacggtgcaatagagagggtagactgcctgagcctggacaggtcgtggtgtacgagcctgagaagccagccagaatgccctggatgcagagatgtgccagcagagcccaagagacactgagaaccctgcctgaggctgatcctctcggactggaagccatcagacggtacttcggcctgatctacgacgtgcaagagctggaccggaaggacatcttcaagcggctgagaggccaggtggacagagacatggtgttcaagttcagagagatcgccaacgacttccggttcatcgacgatgagggcacagccctggtcatcccaacaggacctgaggtggaagatctcgtgcggagactgagaggctgcgagttccctagacctgtgctgcggaaactgcagcagtacagcgtgacagtgcggcacagagagctggaaaagctgagatctgctggcgccgtggaaatgatcggcgacgcttatcccgtgctgagaaacctggccgcctacagcgaagatatgggcctgtgtgtggatagcgtggaagtgtggcagcctgaaggcctggtttctctgggctctgtgggctacccctacgatgtgcctgattacgccggcagctaccctgagttccccaagaaaaagcggaaagtgtga

SEQ ID NO: 169 - DNA, 32 bp, Artificial sequence; identical to SEQ ID NO: 80
  Synthetic

SEQ ID NO: 170 - DNA, 35 bp, Artificial sequence
  Synthetic
agggtcagcttgccgtaggtggcatcgccctcgcc

(19) United States
(12) Patent Application Publication
(10) Pub. No.: US 2024/0229081 A1
(43) Pub. Date: Jul. 11, 2024

(54) CRISPR-CAS3 SYSTEMS FOR TARGETED GENOME ENGINEERING

(71) Applicants: THE REGENTS OF THE UNIVERSITY OF MICHIGAN, Ann Arbor, MI (US); CORNELL UNIVERSITY, Ithaca, NY (US)

(72) Inventors: Yan Zhang, Ann Arbor, MI (US); Zhonggang Hou, Ann Arbor, MI (US); Renke Tan, Ann Arbor, MI (US); Ailong Ke, Ithaca, NY (US)

(21) Appl. No.: 18/563,175

(22) PCT Filed: May 26, 2022
(86) PCT No.: PCT/US22/31091

§ 371 Date: Nov. 21, 2023

Related U.S. Application Data
(60) Provisional application No. 63/193,302, filed on May 26, 2021.

(51) Int. Cl. C12N 15/90; C12N 9/22; C12N 15/11

(52) U.S. Cl. CPC C12N 15/907; C12N 9/22; C12N 15/11; C12N 2310/20; C12N 2800/80

(57) ABSTRACT

The present disclosure provides systems and methods of altering a nucleic acid sequence, which comprise an engineered Type I CRISPR/Cas system comprising Cas3 and Cas11. Particularly. the system and methods described herein use compact engineered CRISPR-Cas3 systems (e.g., Type I-C, Type I-B, or Type I-D) for genetic manipulations (e.g., in eukaryotic cells).

### CROSS-REFERENCE TO RELATED APPLICATIONS

This application claims the benefit of U.S. Provisional Application No. 63/193,302, filed May 26, 2021, the contents of which is herein incorporated by reference in its entirety.

### STATEMENT REGARDING FEDERALLY-SPONSORED RESEARCH

This invention was made with Government support under contract numbers GM117268, GM137833, and GM118174 awarded by the National Institutes of Health. The Government has certain rights in the invention.

### FIELD

The present invention relates to systems and methods for altering nucleic acids. In particular, the present invention relates to engineered Type I CRISPR/Cas systems comprising Cas3 and Cas11 and methods for genome engineering in eukaryotic cells.

### INCORPORATION-BY-REFERENCE OF MATERIAL SUBMITTED ELECTRONICALLY

The text of the computer readable sequence listing filed herewith, titled “39551-601_SEQUENCE_LISTING_ST25”, created May 26, 2022, having a file size of 174,425 bytes, is hereby incorporated by reference in its entirety.

### BACKGROUND

CRISPR-Cas systems employ diverse RNA-guided nucleases to belp microbes fend off bacteriophages and other mobile genetic elements. Current genome editing technologies primarily use single effector enzymes such as Cas9 or Cas12 from Class II CRISPR systems, for programmable DNA sequence alterations. Cas9 or Cas12 is guided by its CRISPR RNA (crRNA) to find the complementary target site flanked by a short protospacer-adjacent motif (PAM), and then cleaves the DNA at precise locations. The highly prevalent Class I Type I CRISPR has only begun to be harnessed for eukaryotic genome engineering recently. Unlike Cas9, Type I CRISPR interference requires coordinated action of a multi-subunit ribonucleoprotein (RNP) complex Cascade that seeks out a PAM-flanked target site, and a helicase-nuclease enzyme Cas3 that is recruited to the resulting R-loop and processively shred the invader's DNA. Due to this unique feature, CRISPR-Cas3 holds great potential for numerous eukaryotic applications, such as targeted deletion of large chromosomal regions, interrogation of non-coding elements, removal of integrated viral genomes, as well as prokaryotic genome minimization, and removal of prophages, pathogenicity islands, or gene clusters, and the like.

Type I system is the most widespread and diversified type of CRISPR and is further classified into eight subtypes (I-A through I-F, I-Fv, and I-U) based on cas gene composition. Since 2019, Cascade-Cas3 has been repurposed to efficiently create targeted large chromosomal deletions of up to 30-100 kilobases (kb) in human cells. In addition, Cascade fusions with FokI nuclease or other effector domains have also enabled programmable transcription modulation in human cells, mammalian gene targeting, and gene activation in plants. These applications mainly focused on four different Type I-E CRISPR-Cas systems from Thermobifida fusca (Tfu), Escherichia coli (Eco), Pseudomonas aeruginosa (Pse), and Streptococcus thermophilus (Sth) that all prefer similar 5′-AAG or 5′-AA PAM sequences; although examples based on other subtypes also exist (e.g., Listeria monocytogenes I-B, Microcystis aeruginosa I-D, and Pseudomonas aeruginosa I-F). Genetic engineering by Type I-E Cascade-Cas3 requires 6 cas genes and a CRISPR array, totaling 7-8 kb in size which is 60-80% larger than the commonly used Streptococcus pyogenes Cas9. Such complexity and relatively large gene size could hinder in vivo delivery using viral vectors that have cargo size constraints. To date, the most streamlined CRISPR-Cas3 systems that belong to Type I-C have never been exploited for eukaryotic use, despite the recent adoption of Pseudomonas aeruginosa I-C system for targeted large deletion of up to 424 kb from bacterial genomes. Nonetheless, most Type I CRISPRs remain untapped for biotechnology.

### SUMMARY

Provided herein are systems for altering a target nucleic acid sequence comprising an engineered Clustered Regularly Interspaced Short Palindromic Repeats (CRISPR)-CRISPR associated (Cas) (CRISPR-Cas) system, and/or one or more nucleic acids encoding the engineered CRISPR-Cas system. The engineered CRISPR-Cas system comprises: Cas11; Cas3; two or more additional Cas proteins from a CRISPR-Associated Complex for Anti-viral Defense (Cascade) complex: and at least one guide RNA (gRNA), wherein each gRNA is configured to hybridize to a portion of a target nucleic acid sequence. In some embodiments, the two or more additional Cas proteins are selected from the group consisting of Cas5, Cas7, Cas6, and Cas8 or Cmx8. In some embodiments, the system further comprises at least one target nucleic acid.

In some embodiments, the one or more nucleic acids comprises one or more messenger RNAs. one or more vectors, or a combination thereof. In some embodiments, Cas11, Cas3, and the two or more additional Cas proteins are encoded by a single nucleic acid. In some embodiments, the two or more additional Cas proteins are encoded by different nucleic acids. In some embodiments, the guide RNA is encoded by a different nucleic acid than Cas 11, Cas3, the two or more additional Cas proteins, or a combination thereof. In some embodiments, the guide RNA, Cas 11, Cas3, and the two or more additional Cas proteins are encoded by a single nucleic acid. In some embodiments, at least one or all of Cas11. Cas3, and the two or more additional Cas proteins comprise a nuclear localization sequence or a tag.

In some embodiments, the engineered CRISPR-Cas system is derived from a Type I CRISPR-Cas system. In some embodiments, the Type I CRISPR-Cas system is Type I-B, Type I-C, or Type I-D system. In some embodiments, the system is derived from Neisseria lactamica.

In some embodiments, the system comprises Cas11, Cas3, Cas5, Cas6, Cas7, and Cmx8. In some embodiments, the system comprises Cas11, Cas3, Cas5, Cas6, Cas7, and Cas10. In some embodiments, the system comprises Cas11, Cas3, Cas5, Cas7, and Cas8.

In some embodiments, the at least one gRNA is encoded in a CRISPR RNA (crRNA) array. In some embodiments, the at least one gRNA comprises a non-naturally occurring gRNA.

In some embodiments, the system comprises two or more engineered CRISPR-Cas systems or one or more nucleic acids encoding two or more engineered (CRISPR-Cas) systems. In some embodiments, the two or more engineered CRISPR-Cas systems are derived from different subtypes of Type I CRISPR-Cas systems. In some embodiments, the two or more engineered CRISPR-Cas systems comprise two Type I CRISPR-Cas systems selected from the group consisting of: a Type I-B CRISPR-Cas system, a Type I-C CRISPR-Cas system, and a Type I-D CRISPR-Cas system.

Also provided herein are cells comprising the disclosed systems. In some embodiments, the cell is a eukaryotic cell.

Further provided are methods of altering a target nucleic acid sequence comprising contacting a target nucleic acid sequence with the disclosed systems or a composition thereof.

In some embodiments, altering a target nucleic acid sequence comprises deletion of the target nucleic acid sequence. In some embodiments, the deletion is unidirectional. In some embodiments, the deletion comprises from about 500 nucleotides to about 100,000 nucleotides (e.g., about 5,000 nucleotides to about 20,000 nucleotides).

In some embodiments, the target nucleic acid sequence encodes a gene product. In some embodiments, the target nucleic acid sequence is a genomic DNA sequence. In some embodiments, the target nucleic acid sequence is in a cell. In some embodiments, the cell is a eukaryotic cell (e.g., a mammalian cell, a human cell).

In some embodiments, contacting a target nucleic acid sequence comprises introducing the system into the cell. In some embodiments, introducing the system into the cell comprises administering the system to a subject (e.g., a human). In some embodiments, the administering comprises in vivo administration. In some embodiments, the administering comprises transplantation of ex vivo treated cells comprising the system.

Other aspects and embodiments of the disclosure will be apparent in light of the following detailed description and accompanying figures.

### BRIEF DESCRIPTION OF THE DRAWINGS

FIGS. 1A-1G show a compact CRISPR-Cas3 from N. lactamica conferred plasmid immunity in bacteria. FIG. 1A is a schematic of the miniature type I-C CRISPR-Cas locus from N. lactamica, with cas1, cas2 and cas4, cas7, cas8, cas5, and cas3. Black rectangles, CRISPR repeats; diamonds, CRISPR spacers. Cas genes are drawn to scale, while the CRISPR array is enlarged for clarity. FIG. 1B is an informatic prediction defining a 5′-TTC PAM. Potential natural targets for native spacers of N. lactamica ATCC 23970 were defined using CRISPRTarget, with up to 1 nt mismatch in spacer-target complementarity allowed and denoted in bold and red (SEQ ID NOs: 19-41). The target sequences and their 10-nt flanks on both 5′ (SEQ ID NOs: 1-18) and 3′ (SEQ ID NOs: 42-64) sides were aligned using Weblogo, and the resulting sequence logos were shown at the top. FIG. 1C is a schematic overview of the plasmid interference assay in E. coli. BL21-AI derivative strains harboring four plasmids encoding crispr, cas3, cascade genes, and a target-PAM sequence were cultured with or without induction of crispr-cas expression, serial diluted, and plated on LB plates with triple or quadruple antibiotics to track cell survival. Reduced colony count on quadruple antibiotics plate for the induced culture indicates a CRISPR interference phenotype. FIG. 1D is a representative image of an interference assay where isogenic E. coli strains were titered on a quadruple antibiotics plate in 10-fold serial dilutions. Under induced conditions, a matching target with 5′-TTC PAM led to drastic reduction in colony counts compared to the empty target control, indicative of robust CRISPR interference in vivo. FIG. 1E is a graph of the induction of crispr-cas expression which led to robust interference for three different targets flanked by a 5′ TTC PAM, but not for the controls containing either no target or a 5′AAG-flanked target. Depletion ratio was calculated as the colony-forming units (CFUs) from triple antibiotic control plate divided by CFUs from quadruple antibiotic test plate. Data are displayed as log scale plots of the mean depletion ratio±SEM, n=3. FIG. 1F is a schematic of the crispr-cas loci in isogenic mutant strains used in FIG. 1G. FIG. 1G is a graph of CRISPR Interference mediated by the Nla type I-C system utilizing the cas7, cas8, cas5, cas3 and crispr genes but not cas4. Data are quantified and shown as in FIGS. 1D and 1E.

FIGS. 2A-2H show N. lactamica CRISPR-Cas3 RNP achieved high-efficiency multiplexed genome editing in human cells. FIG. 2A is an SDS-PAGE of purified Nla Cas3 protein and Cascade RNP samples targeting different genes (GFP-G2, tdTomato (tdTm), and HPRT1&2). Star, an unexpected small peptide that is consistently co-purified with NlaCascade and further examined in FIG. 4. Cascade tdTomato (tdTm) was purified using a slightly different strategy, and therefore contains an extra band of ˜68 KDa corresponding to His-MBP-Cas5. FIG. 2B is a schematic of the hESC dual-reporter cells used in FIGS. 2C-2D, with protospacers for the EGFP− or tdTm− targeting Cascade and corresponding PAMs as indicated. FIG. 2C is a graph of Cascade RNP targeting either EGFP, tdTm or a control locus (Non-targeting (NT), e.g., HPRT1) electroporated into hESCs with or without purified Cas3. The gene editing efficiency was shown as the percentage of EGFP−/tdTm+ (white bar) or tdTm−/EGFP+ (shaded grey bar) cells in the total population. Data are shown as mean±SEM, n=3. FIG. 2D is representative flow cytometry plots from experiments in FIG. 2C, with percentages of EGFP− /tdTm+ or EGPF+/tdTm− cells shown on the top or to the right, respectively. FIG. 2E is a graph of robust multiplexed RNP editing in HAP1 dual reporter cells. Cascade RNP purified using each multi-spacer CRISPR array depicted at the bottom was electroporated into HAP1 reporter cells together with Cas3. The reporter gene editing efficiencies were shown as the percentage of GFP−/tdTm+ (black bar), tdTm−/GFP+ (light grey bar), and GFP−/tdTm− (dark grey bar) cells in the total population. Data are shown as mean±SEM, n=3. The green, red, purple, and yellow spacers represent Nla guides targeting EGFP, tdTm, HPRT, and CCR5 genes, respectively. FIG. 2F is a schematic of the HPRT1 locus in HAP1 cells, with protospacers for the two HPRT-targeting Cascades and corresponding PAMs as indicated. FIG. 2G is a chart of the HPRT1 editing efficiency measured by single clone 6-TG survival assay. The survival rate is the ratio between the average colony counts of 6-TG+ vs. 6-TG− conditions. FIG. 2H is a bar graph plotting the colony counts from FIG. 2G. Data are shown as mean±SEM, n=3.

FIGS. 3A-3E show NlaCRISPR-Cas3 generated targeted, large, and unidirectional DNA deletions. FIG. 3A is a schematic of the HPRT1 locus and annealing sites for PCR primers used in FIGS. 3B, 3D, and 3E. All positions indicated are relative to HPRT1 translation start site (+1). The dashed line marks the recognition site (3rd nt of the TTC PAM) for guide HPRT1-G1. Arrow, presumed direction of NlaCas3 translocation. FIGS. 3B, 3D, and 3E show the characterization of genomic lesions by long-range PCRs, using primers amplifying regions downstream (FIG. 3B) or upstream (FIG. 3D) of the CRISPR-targeted site, or regions spanning both directions (FIG. 3E). A spectrum of large, unidirectional deletions was detected in the PAM-proximal genomic region, from cells treated with Cas3 and Cascade HPRT-G1, but not the untreated control (no RNP) cells. PCR primers used are listed and their annealing sites depicted in FIG. 3A. Smaller-than-full-length amplicons indicate large genomic deletions. M, DNA size markers. FIG. 3C is a schematic of the deletion locations at the HPRT1 locus, revealed by TOPO cloning of pooled tiling PCRs from lanes 6-10 in FIG. 3B and Sanger sequencing. Black lines, deleted genomic regions.

FIGS. 4A-4H show Cas11, a hidden product from internal translation, facilitated robust RNP editing with NlaCRISPR-Cas3. FIG. 4A is schematics of five plasmids used in FIGS. 4B and 4C, to express NlaCRISPR-Cas3 components in human cells. Rectangles indicate EF1a promoter (EF1a), HA tag, NLS, bGH poly A signal (pA), and U6 promoter. The five crispr-cas plasmids from FIG. 5A were co-transfected into HAP1 reporter cells to evaluate genome editing efficiency. The editing rates were shown as the percentage of GFP-cells in FIG. 4B. G1 through G4, four different CRISPR guides targeting 5′-TTC flanked sites in EGFP; their sequences and locations are depicted in FIG. 12A. A SpyCas9 plasmid targeting EGFP was included as the positive control. FIG. 4C is representative flow cytometry plots of experiments in FIG. 4B, with percentages of EGFP-cells in the population shown on the top. FIG. 4D is schematics of the Nla cas8 and cas11 genes. Blue, the predicted RBS and internal translational start site for cas11: orange, the putative Cas11 protein: purple, mutations introduced to create the Δcas11 construct. The first six amino acids of the ˜14 kDa band obtained through Edman degradation were marked by the orange line. FIG. 4E is schematics of plasmids used for the expression and purification of Δcas11 and cas11-rescued versions of NlaCascade in FIGS. 4F-4G. FIG. 4F is SEC chromatograms of NlaCascade RNPs purified via an N-terminal His tag on Cas7. Elution profiles of wt, Δcas11, and cas11-rescued NlaCascade RNP samples are displayed as black, dashed gray, and orange lines. FIG. 4G is SDS-PAGE of purified NlaCascade from FIG. 4F. FIG. 4H is a graph of Cas11-rescued Cascade RNP mediating genome editing as the wt counterpart. Editing efficiencies were measured by flow cytometry and shown as the percentage of GFP negative cells in total population. Data in FIGS. 4B and 4H are shown as mean±SEM, n=3.

FIGS. 5A-5E show Cas11 enabled efficient plasmid- and mRNA-based editing by NlaCRISPR-Cas3. FIG. 5A is schematics of the six plasmids used in FIGS. 5B and 5C. A separate Nlacas11-encoding plasmid is included, the rest are as in FIG. 4A. FIG. 5B is a graph of the gene editing efficiencies for the crispr-cas plasmids from FIG. 5A transfected into HAP1 reporter cells. Gene editing efficiencies were evaluated and plotted as described in FIG. 4B. The equal ratio mix contains equal amounts of plasmids for each cascade subunit, whereas the optimized mix has more Cas8 and less Cas5. FIG. 5C is representative flow cytometry plots of experiments in FIG. 5B, with percentages of EGFP- cells in the population shown on the top. FIG. 5D is schematics of the cas mRNAs, pre-CRISPR RNA and pCR plasmid used. Green, GFP-targeting CRISPR spacer. FIG. 5E is gene editing efficiencies of mRNAs encoding NlaCascade components with or without Cas11 electroporated into HAP1 reporter cells, along with a GFP-targeting CRISPR in the form of pre-CRISPR transcript (RNA) or plasmid (DNA). Gene editing efficiencies were plotted as described in FIG. 4B. Data in FIGS. 5B and 5E are shown as mean±SEM, n=3.

FIGS. 6A-6E show Cas11 established diverse miniature CRISPR-Cas3 orthologs as gene editors. FIG. 6A is a phylogenetic tree of the large subunit gene cas8 or cas10, from selective type 1 CRISPR systems analyzed for editing in human cells. The Tfu and Eco I-E systems are included for comparison. FIGS. 6B-6E are schematics of the CRISPR-Cas3 loci (top) and gene editing efficiencies (bottom) of the Bha I-C (FIG. 6B), Dvu I-C (FIG. 6C), Syn I-D (FIG. 6D), and Syn I-B (FIG. 6E) systems, respectively. Editing rates were measured and shown as in FIG. 4B, except that for the Syn I-B system in FIG. 6E editing is measured as tdTm- cells in the population. Data in FIGS. 6B-6E are shown as mean±SEM, n=3 or 4.

FIGS. 7A-7E show CRISPR-Cas3 orthogonality in human cells. FIG. 7A is the PAM and repeat sequences of the CRISPR-Cas3 systems used, with the lengths of their spacers and repeats (Nla I-C repeat is SEQ ID NO: 78; Bha I-C repeat is SEQ ID NO: 79; Dvu I-C repeat is SEQ ID NO: 80; Syn I-B repeat is SEQ ID NO: 81; Syn I-D repeat is SEQ ID NO: 82) indicated. FIGS. 7B and 7D are graphs from mix-and-match experiments assaying Cas plasmids from three different type I systems paired with each other's CRISPR construct. Three distinct I-C editors are analyzed in FIG. 7B, while the Nla I-C, Syn I-D, and Syn I-B systems are tested in FIG. 7D, respectively. Gene editing efficiencies were evaluated in HAP1 reporter cells and plotted as described in FIG. 6. Data are shown as mean±SEM, n=3. FIGS. 7C and 7E are heatmaps of gene editing efficiencies reported in FIGS. 7B and 7D.

FIGS. 8A-8D show Cascade RNP and Cas3 protein titrations in human cell gene editing. FIG. 8A is a graph of RNP editing experiments in HAP1 reporter cells with 50 pmol NlaCas3 and increasing amount of GFP-targeting NlaCascade. Cascade amount electroporated was titrated from 4.5 pmol to 35 pmol. FIG. 8B is a graph of RNP editing in HAP1 reporter cells with 35 pmol GFP-targeting NlaCascade and increasing amount of Cas3. NlaCas3 protein electroporated was 0, 0.2, 0.8. 3.1, 12.5, and 50 pmol. The editing efficiencies in FIGS. 8A-8B were measured and shown as in FIG. 4B. FIGS. 8C-8D are representative flow cytometry plots from experiments in FIG. 8A and FIG. 8B, respectively, with percentages of EGFP- in the total population shown on the top.

FIGS. 9A-9C show NlaCascade-Cas3 RNP enabled gene targeting in multiple human cell lines, at the HPRT1 or CCR5 genomic sites. FIG. 9A is an SDS-PAGE of purified NlaCascade samples used for multiplexed editing in FIG. 2E and for CCR5 targeting. The spacer color scheme is as described in FIG. 2E. FIG. 9B, Top, is a schematic of HPRT1 locus. Big black arrows, annealing sites for two primers used in genomic PCR. All positions indicated are relative to HPRT1 translation start site (+1). The blue dashed line marks the recognition site (3rd nt of the TTC PAM) for guide HPRT1-G1. Blue hatched arrow, presumed direction of NlaCas3 translocation. FIG. 9B, Bottom, shows long-range PCR using genomic DNA extracted from various human cell types (HAP1, hESCs, HEK293T, and Hela) edited with Cas3 and HPRT1-targeting Cascade RNP. Smaller-than-full-length amplicons indicate large genomic deletions caused by HPRT1 targeting. M, DNA size markers. FIG. 9C, Left, is a schematic of CCR5 locus. Big black arrows, annealing sites for two primers used in genomic PCR. All positions indicated are relative to CCRS translation start site (+1). The blue dashed line marks the recognition site (3rd nt of the TTC PAM) for guide CCR5-G2. Blue batched arrow, presumed direction of NlaCas3 translocation. FIG. 9C, Right, is long-range PCR as described in FIG. 9A, using genomic DNA extracted from HAP1 cells edited with Cas3 and CCR5-targeting Cascade RNP. Smaller-than-full-length amplicons indicate large genomic deletions resulted from successful CCR5 targeting.

FIGS. 10A-10E show Nla CRISPR-Cas3 generated targeted, large unidirectional genomic deletions in hESC and HEK293T cells. FIG. 10A is a schematic of HPRT1 locus and annealing sites of PCR primers used in FIGS. 10B, 10D, and 10E. All positions indicated are relative to HPRT1 translation start site (+1). The blue dashed line marks the recognition site (3rd nt of the TTC PAM) for guide HPRT1-G1. Blue hatched arrow, presumed direction of NlaCas3 translocation. FIGS. 10B, 10D, and 10E show genomic lesion analysis via long-range PCRs, using primers amplifying regions downstream (FIG. 10B) or upstream (FIG. 10D) of the CRISPR-targeted HPRT site, or regions spanning both directions (FIG. 10E). The genomic DNA samples used as PCR template were extracted from hESCs and HEK293T cells. A spectrum of large, unidirectional deletions was detected in the PAM-proximal genomic region, from cells treated with Cas3 and Cascade HPRT-G1, but not the untreated control (no RNP) cells. Smaller-than-expected-full-length amplicons indicate large DNA deletions. The lack of full-length PCR product from the un-edited control is likely due to a GC-rich region in exon 1 (˜400 bp downstream of the target site) that prevents PCR amplification. Smaller-than-full-length amplicons indicate large genomic deletions. M, DNA size markers. FIG. 10C is a schematic of HPRT1 deletion locations, revealed by TOPO cloning of pooled tiling PCRs from lanes 6-10 in FIG. 10B and Sanger sequencing of randomly selected individual clones. Black lines, deleted genomic regions. Orange, green and the lack of dots on the right indicate deletion junctions—orange represents one deletion with a small insertion or partial inversion and green represents two deletions.

FIGS. 11A-11E show Nla CRISPR-Cas3 induced large deletions at the DNMT3b locus in hESCs. FIG. 11A is a schematic of DNMT3b-EGFP locus in hESC reporter cell line. Annealing sites of PCR primers used in FIGS. 11B-11E are indicated. All positions indicated are relative to EGFP translation start site (+1). The blue dashed line marks the recognition site (3rd nt of the TTC PAM) for guide EGFP-G2. Blue hatched arrow, presumed direction of NlaCas3 translocation. FIGS. 11B-11D show genomic lesion analysis via long-range PCRs, using primers amplifying regions downstream (FIG. 11B) or upstream (FIG. 11C) of the CRISPR-targeted GFP site, or regions spanning both directions (FIG. 11D). Genomic DNA used as PCR template was extracted from a hESC reporter line bearing EGFP and tdTm at the endogenous DNMT3b locus. A spectrum of large, unidirectional deletions was detected in the PAM-proximal region, from cells edited with Cas3 and Cascade GFP-G2, but not “no RNP” control cells. Smaller-than-expected-full-length amplicons indicate large DNA deletions. M, DNA size markers. Discontinuous lanes from the same gel are separated by the dashed grey line. FIG. 11E is a schematic of deletion locations revealed by TOPO cloning of pooled tiling PCRs from lanes 5-8 from FIG. 11B and 19-20 from FIG. 11D. Randomly selected individual clones are Sanger sequenced. Black lines, deleted genomic regions. Orange, green and the lack of dots on the right indicate deletion junctions—orange represents one deletion with a small insertion or partial inversion and green represents two deletions. Note the existence of three bidirectional deletion events from PCR of lanes 19-20.

FIGS. 12A-12F show Cas11 is the component facilitating efficient plasmid-based editing in human cells with Nla CRISPR-Cas3. FIG. 12A is schematics of the EGFP reporter and target sites for all NlaCascade RNP and SpyCas9. Sequences for protospacers are indicated in blue and corresponding PAMs in magenta. FIG. 12B is anti-HA western blot detecting expression of all canonical cas genes of Nla I-C CRISPR system (cas5, cas7, cas8 and cas3) after plasmid transfection into HAP1 cells. Bottom, GAPDH is probed as loading control. Molecular weight markers (kDa) are indicated. FIG. 12C shows that the Nla I-C CRISPR system indeed expresses a previously overlooked cas11 gene from within cas8. Plasmids expressing CRISPR and the cascade operon were co-transformed into E. coli BL21 (DE3), and the resulting strains were subject to western blot analysis. The pCascade plasmids have a Flag-tag at the C-terminus of cas8. Both Cas8 and Cas11 proteins were detected by anti-Flag western from the wt strain; whereas the Cas11 production was abolished by mutations introduced to the RBS and alternative translation start site in cas8. Molecular weight markers (kDa) are indicated. FIG. 12D shows the gene editing efficiencies for GFP-targeting guides 2, 3, and 4 from the Nla crispr-cas plasmids depicted in FIG. 5A were transfected into HAP1 reporter cells. The results were plotted as the percentage of EGFP-cells in the total population. Data are shown as mean±SEM, n=3. FIG. 12E is schematics of the pCascade polycistronic expression constructs tested in FIG. 12F. The NLS, HA tag and regulatory elements are as described in FIG. 4A. Each plasmid depicted in FIG. 12E was transfected into HAP1 reporter cells along with the Cas3- and CRISPR-encoding plasmids, and the gene editing efficiencies were assessed and shown in FIG. 12F as in FIG. 4B. Data are shown as mean±SEM, n=3.

FIGS. 13A-13C show target sequences and protein expression analyses for Dvu I-C, Syn I-D, and Syn I-B CRISPR systems. FIGS. 13A-13C, Top are schematics of the target sites used for the Dvu I-C (FIG. 13A), Syn I-D (FIG. 13B), and Syn I-B (FIG. 13C) CRISPR-Cas respectively, with protospacers for the reporter-targeting Cascade RNPs indicated in blue and corresponding PAMs in magenta. FIGS. 13A-13C, Bottom are anti-HA western blot detecting expression of all cas genes of the Dvu I-C (FIG. 13A), Syn I-D (FIG. 13B), and Syn I-B (FIG. 13C) systems, after transfecting the corresponding plasmids into HAP1 cells. GAPDH was probed as the loading control. Molecular weight markers (kDa) are indicated.

FIGS. 14A-14C show repeat specificity for CRISPR-Cas3 orthogonality in buman cells. FIG. 14A is a schematic of wild-type CRISPR constructs used for the Nla I-C, Syn I-D, and Syn I-B editors. Light grey, dark grey, and black rectangles indicate CRISPR repeats of the I-C, I-B, and I-D systems, respectively. Light green (EGFP-targeting), red (tdTm-targeting), and dark green (EGFP-targeting) diamonds indicate CRISPR spacers used for the I-C, I-B, and I-D editors. FIG. 14B shows mix-and-match experiments assaying Cas plasmids from the three distinct type I systems paired with wt or chimeric CRISPR constructs. The actual repeat and spacer analyzed in each test were indicated, with schematics of the entire CRISPR array included at the bottom; the three wt CRISPRs without repeat swap were boxed. Genome editing was evaluated in HAP1 reporter cells following plasmid transfection, and the efficiencies were plotted as described in FIG. 4B. Data are shown as mean±SEM, n=3. FIG. 14C is heatmaps of gene editing rates reported in FIG. 14B.

FIGS. 15A and 15B show comprehensive PAM profile determination for Nla type I-C CRISPR in bacteria and in extracts. FIG. 15A is the analysis of all 64 possible 5′-NNN PAM variants using E. coliplasmid interference assay as described in FIG. 1C. Induction of crispr-cas expression led to >100-fold interference for targets flanked by twelve different PAM variants. These 12 potentially functional PAMs in bacteria are TTC, CCC, CTA, CTC, CTT, TCA, TCC, TCT, TCG, TTA, TTT, TTG. Depletion ratios were calculated as the colony-forming units (CFUs) from the triple antibiotic control plate divided by CFUs from the quadruple-antibiotic test plate for the same sample. Data are displayed as log scale plots of the mean depletion ratio±SEM, n=3. Target seq used is NNN PAM+ AGGGTCAGCTTGCCGTAGGTGGCATCGCCCTCGCC (a GFP-targeting spacer; SEQ ID NO: 170). FIG. 15B is Krona plots of PAM profile for N. lactamica I-C CRISPR-Cas determined using PAM-DETECT, a cell-free transcription-translation systems (TXTL)-based assay, as described in Wimmer et al., Mol Cell. 2022 Mar 17:82(6): 1210-1224.e6. In this assay, Cascade is directed to bind to target DNA flanked by a library of potential PAM variants. Only functional PAMs bound by Cascade will lead to protection of target sequence from restriction enzyme digestion. The functional PAMs defined are listed, with frequencies of their occurrence in the final enriched library shown in parenthesis. The most robust PAM group includes TTC, CTC, TCC, TTT, TTG.

FIG. 16 shows the validation of top functional PAMs for Nla type I-C CRISPR system in buman cell gene editing. Top five PAMs (TTC, TCC, CTC, TTG, TTT) and selective negative control PAMs (AAG, TGT) defined in FIG. 15 were assayed for gene editing in a human cell GFP-reporter line. Mixture of CRISPR-Cas plasmids were co-transfected into HAP1-GFP reporter cells to evaluate genome-editing efficiency. The editing efficiencies are shown as the percentage of EGFP-negative cells. Data are shown as mean±SD. For each top five PAMs, five different target sites within GFP ORF were tested. For each negative control PAM, one GFP target site was included.

### DETAILED DESCRIPTION OF THE INVENTION

The present disclosure is directed to a Type I CRISPR system repurposed for eukaryotic genome manipulation and a framework to systematically implement divergent and compact CRISPR-Cas3 editors.

Type I CRISPRs from subtypes I-C, I-B, and I-D together encompass nearly a quarter of all native CRISPRs. Type I-C is the most streamlined, requiring only 4 cas genes (cas3-cas5-cas7-cas8) and l CRISPR for DNA targeting (total gene size ˜5-6 kb). Types I-B and I-D each require five cas genes (cas3-cas5-cas6-cas7-cas8 for I-B, and cas3-cas5-cas6-cas7-cas10 for I-D).

As described herein, a previously unannotated cas11 gene encoded by internal translation from within Nlacas8 which produces a small subunit of Cascade in bacteria was identified. The resulting ˜14 kDa NlaCas11 protein is a subunit of Cascade integral for stable Cascade complex formation. Supplying Cas11 using a separate mammalian expression cassette enabled robust plasmid- or mRNA-based based editing in mammalian cells. This strategy was applicable to establish divergent compact CRISPR-Cas3 editors across the 1-B, I-C and I-D subtypes and allowed orthogonal systems to be used in a single cell.

### 1. Definitions

To facilitate an understanding of the present technology, a number of terms and phrases are defined below. Additional definitions are set forth throughout the detailed description.

The terms “comprise(s),” “include(s),” “having.” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non- nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%. 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt′s solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C. (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tm of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46: 453 (1960) and Doty et al., Proc. Natl. Acad. Sci. USA, 46: 461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) may also be considered a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “variant” refers to the exhibition of qualities that have a pattern that deviates from what occurs in nature. In some embodiments, a variant may also be a mutant.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein binding protein). In the case of a protein domain-binding protein, it can bind to itself (to form homodimers, homotrimers, etc.) and/or it can bind to one or more molecules of a different protein or proteins.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms). Alternatively, DNA sequences encoding RNA (e.g., DNA-targeting RNA) that is not translated may also be considered recombinant. Thus, the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention but may be a naturally occurring amino acid sequence.

A “vector” or “expression vector” is a replicon, such as plasmid, phage, virus, or cosmid, to which another DNA segment, e.g., an “insert.” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults, juveniles (e.g., children), or infants. Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats: laboratory animals including rodents, such as rats, mice and guinea pigs, and the like. Examples of non-mammals include, but are not limited to, birds, fish, and the like. In one embodiment, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. CRISPR/Cas System for Altering a DNA Sequence

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Transcription of a CRISPR locus produces a “pre-crRNA,” which is processed to yield crRNAs containing spacer-repeat fragments that guide effector nuclease complexes to cleave dsDNA sequences complementary to the spacer. Several different types of CRISPR systems are known, (e.g., type I, type II, or type III), and classified based on the Cas protein type and the use of a proto-spacer-adjacent motif (PAM) for selection of proto-spacers in invading DNA.

Engineering CRISPR/Cas systems for use in eukaryotic cells typically involves reconstitution of the CRISPR/Cas complex. Typically, the RNA sequences necessary for CRISPR/Cas systems are referred to collectively as “guide RNA” (gRNA) or single guide RNA (sgRNA). Thus, the terms “guide RNA,” “single guide RNA,” and “synthetic guide RNA,” are used interchangeably herein and may refer to a nucleic acid sequence comprising a tracrRNA and a pre-crRNA array containing a guide sequence. The terms “guide sequence,” “guide,” and “spacer,” are used interchangeably herein and refer to the nucleotide sequence within a guide RNA that specifies the target site.

The system disclosed herein comprises an engineered Clustered Regularly Interspaced Short Palindromic Repeats (CRISPR)-CRISPR associated (Cas) (CRISPR-Cas) system, and/or one or more nucleic acids encoding the engineered CRISPR-Cas system, wherein the engineered CRISPR-Cas system comprises: (a) Cas11; (b) Cas3; (c) two or more additional Cas proteins from CRISPR-Associated Complex for Anti-viral Defense (Cascade) complex: and (d) at least one guide RNA (gRNA), wherein each gRNA is configured to hybridize to a portion of a target nucleic acid sequence.

The terms “Cascade (CRISPR-Associated Complex for Anti-viral Defense)” or “Cascade complex” as used herein, refer to a ribonucleoprotein complex comprised of multiple protein subunits (e.g., Cas proteins) used naturally in bacteria as a mechanism for nucleic acid-based immune defense. The Cascade complex recognizes nucleic acid targets via direct base-pairing to guide RNA contained in the complex. Acceptance of target recognition by Cascade results in a conformational change which, in E. coli and other bacteria, recruits a protein component referred to as Cas3. Cas3 may comprise a single protein unit which contains helicase and nuclease domains. After target validation by Cascade, Cas3 nicks the strand of DNA that is looped out by the R-loop formed by Cascade approximately 9-12 nucleotides inward from the PAM site. Cas3 then uses its helicase/nuclease activity to processively degrade substrate nucleic acids, moving in a 3′ to 5′ direction. In some embodiments, the two or more additional Cas proteins from the Cascade complex are selected from the group consisting of Cas5, Cas7, Cas6, and Cas8 or Cmx8.

The engineered CRISPR-Cas system may be derived from a CRISPR-Cas system of any type or subtype. In some embodiments, the engineered CRISPR-Cas system is derived from a Type I CRISPR-Cas system. Type I system is the most widespread and diversified type of CRISPR and is further classified into eight subtypes (1-A through I-F, I-Fv, and I-U) based on cas gene composition. For example, subtypes I-E and I-F lack the cas4 gene.

In some embodiments, the Type I CRISPR-Cas system is a Type I-C system. Elements or sequences from any suitable Type I-C CRISPR-Cas system may be used in the context of the disclosed methods. In some embodiments, the system comprises Cas11, Cas3, Cas5, Cas7, and Cas8.

In some embodiments, the Type I-C CRISPR-Cas system may be derived from CRISPR-Cas elements (e.g., Cascade-Cas3 proteins or variants thereof) from a Neisseria species (e.g., Neisseria lactamica). The genus Neisseria comprises many gram-negative β-proteobacteria that interact with eukaryotic hosts, but only two organisms, the gonococcus (Gc) and its close relative the meningococcus (Mc), are human pathogens, both of which colonize mucosal surfaces. Many non-pathogenic Neisseria species also colonize the human nasopharynx, and among them N. lactamica is the most widely studied commensal bacterium. In some embodiments, the CRISPR-Cas system used in the context of the present disclosure is derived from the Type I-C system of Neisseria lactamica (Nla), or variants thereof.

N. lactamica Type I-C proteins may comprise the wild-type amino acid sequence or variant having an amino acid sequence that is at least about 85% identical (e.g., about 86%, about 87%, about 88%, about 89%, about 90%, about 91%, about 92%, about 93%, about 94%, about 95%, about 96%, about 97%, about 98%, about 99%, or about 100%) to the amino acid sequence of any protein of the N. lactamica Type I-C proteins. The N. lactamica Type I-C proteins may be those as disclosed in International Patent Application No. PCT/US21/034165, incorporated herein by reference in its entirety.

In certain embodiments, the Cas3 protein is encoded by a nucleic acid sequence having at least 70% similarity to that of SEQ ID NO: 99 or SEQ ID NO: 100, the Cas5 protein is encoded by a nucleic acid sequence having at least 70% similarity to that of SEQ ID NO: 102 or SEQ ID NO: 103, the Cas8 protein is encoded by a nucleic acid sequence having at least 70% similarity to that of SEQ ID NO: 105 or SEQ ID NO: 106, the Cas7 protein is encoded by a nucleic acid sequence having at least 70% similarity to that of SEQ ID NO: 108 or SEQ ID NO: 109, and a Cas11 protein is encoded by the nucleic acid sequence having at least 70% similarity to that of SEQ ID NO: 111 or SEQ ID NO: 112.

In certain embodiments, the Cas3 protein is encoded by the nucleic acid sequence of SEQ ID NO: 99 or SEQ ID NO: 100, the Cas5 protein is encoded by the nucleic acid sequence of SEQ ID NO: 102 or SEQ ID NO: 103, the Cas8 protein is encoded by the nucleic acid sequence of SEQ ID NO: 105 or SEQ ID NO: 106, the Cas7 protein is encoded by the nucleic acid sequence of SEQ ID NO: 108 or SEQ ID NO: 109, and the Cas11 protein is encoded by the nucleic acid sequence of SEQ ID NO: 111 or SEQ ID NO: 112. However, the invention is not limited to these exemplary sequences. Indeed, genetic sequences can vary between different strains, and this natural scope of allelic variation is included within the scope of the invention.

In certain embodiments, the Cas3 protein comprises the amino acid sequence of SEQ ID NO: 101, the Cas5 protein comprises the amino acid sequence of SEQ ID NO: 104, the Cas8 protein comprises the amino acid sequence of SEQ ID NO: 107, the Cas7 protein comprises the amino acid sequence of SEQ ID NO: 110, and the Cas11 protein comprises the amino acid sequence of SEQ ID NO: 113. However, the invention is not limited to these exemplary sequences. For example, in certain embodiments, the Cas3 protein comprises an amino acid sequence having at least 70% similarity to that of SEQ ID NO: 101, the Cas5 protein comprises an amino acid sequence having at least 70% similarity to that of SEQ ID NO: 104, the Cas8 protein comprises an amino acid sequence having at least 70% similarity to that of SEQ ID NO: 107, the Cas7 protein comprises an amino acid sequence having at least 70% similarity to that of SEQ ID NO: 110, and the Cas11 protein comprises an amino acid sequence of SEQ ID NO: 113.

In some embodiments, the Type I-C CRISPR-Cas system is derived from CRISPR-Cas elements (e.g., Cascade-Cas3 proteins or variants thereof) from a Bacillus species (e.g., Bacillus halodurans (Bha)) system, or variants thereof. The genus Bacillus is a diverse group of spore-forming bacteria ubiquitous in the environment. Bacillus anthracis, the agent of anthrax, is the only obligate Bacillus pathogen in vertebrates. Bacillus larvae, B lentimorbus, B popilliae, B sphaericus, and B thuringiensis are pathogens of specific groups of insects. A number of other species, in particular B cereus, are occasional pathogens of humans and livestock, but the large majority of Bacillus species are harmless saprophytes. Thus, the vast majority of Bacillus are nonpathogenic, environmental organisms found in soil, air, dust, and debris. In some embodiments, the CRISPR-Cas system used in the context of the present disclosure is derived from the Type I-C system of Bacillus halodurans (Bha), or variants thereof.

Bacillus halodurans Type I-C proteins may comprise the wild-type amino acid sequence or variant having an amino acid sequence that is at least about 85% identical (e.g., about 86%, about 87%, about 88%, about 89%, about 90%, about 91%, about 92%, about 93%, about 94%, about 95%, about 96%, about 97%, about 98%, about 99%, or about 100%) to the amino acid sequence of any protein of the Bacillus halodurans Type I-C proteins.

In certain embodiments, the Cas3 protein is encoded by a nucleic acid sequence having at least 70% similarity to that of 156, the Cas5 protein is encoded by a nucleic acid sequence having at least 70% similarity to that of SEQ ID NO: 150, the Cas8 (Csd1) protein is encoded by a nucleic acid sequence having at least 70% similarity to that of SEQ ID NO: 152the Cas7 (Csd2) protein is encoded by a nucleic acid sequence having at least 70% similarity to that of SEQ ID NO: 148, and a Cas11 protein is encoded by the nucleic acid sequence having at least 70% similarity to that of SEQ ID NO: 154.

In certain embodiments, the Cas3 protein is encoded by the nucleic acid sequence of SEQ ID NO: 156, the Cas5 protein is encoded by the nucleic acid sequence of SEQ ID NO: 150, the Cas8 (Csd1) protein is encoded by the nucleic acid sequence of SEQ ID NO: 152, the Cas7 (Csd2) protein is encoded by the nucleic acid sequence of SEQ ID NO: 148, and the Cas11 protein is encoded by the nucleic acid sequence of SEQ ID NO: 154. However, the invention is not limited to these exemplary sequences. Indeed, genetic sequences can vary between different strains, and this natural scope of allelie variation is included within the scope of the invention.

In certain embodiments, the Cas3 protein comprises the amino acid sequence of SEQ ID NO: 155, the Cas5 protein comprises the amino acid sequence of SEQ ID NO: 149, the Cas8 (Csd1) protein comprises the amino acid sequence of SEQ ID NO: 151, the Cas7 (Csd2) protein comprises the amino acid sequence of SEQ ID NO: 147, and the Cas11 protein comprises the amino acid sequence of SEQ ID NO: 153. However, the invention is not limited to these exemplary sequences. For example, in certain embodiments, the Cas3 protein comprises an amino acid sequence having at least 70% similarity to that of SEQ ID NO: 155, the Cas5 protein comprises an amino acid sequence having at least 70% similarity to that of SEQ ID NO: 149, the Cas8 (Csd1) protein comprises an amino acid sequence having at least 70% similarity to that of SEQ ID NO: 151, the Cas7 (Csd2) protein comprises an amino acid sequence having at least 70% similarity to that of SEQ ID NO: 147, and the Cas11 protein comprises an amino acid sequence of SEQ ID NO: 153.

In some embodiments, the Type I-C CRISPR-Cas system may be derived from CRISPR-Cas elements (e.g., Cascade-Cas3 proteins or variants thereof) from a Desulfovibrio species (e.g., Desulfovibrio vulgaris (Dvu)) system, or variants thereof. Desulfovibrio is a genus of Gram-negative sulfate-reducing bacteria commonly found in aquatic environments. In some embodiments, the CRISPR-Cas system used in the context of the present disclosure is derived from the Type I-C system of Desulfovibrio vulgaris (Dvu), or variants thereof.

Desulfovibrio vulgaris Type I-C proteins may comprise the wild-type amino acid sequence or variant having an amino acid sequence that is at least about 85% identical (e.g., about 86%, about 87%, about 88%, about 89%, about 90%, about 91%, about 92%, about 93%, about 94%, about 95%, about 96%, about 97%, about 98%, about 99%, or about 100%) to the amino acid sequence of any protein of the Desulfovibrio vulgaris Type I-C proteins.

In certain embodiments, the Cas3 protein is encoded by a nucleic acid sequence having at least 70% similarity to that of 168, the Cas5 protein is encoded by a nucleic acid sequence having at least 70% similarity to that of SEQ ID NO: 160, the Cas8 protein is encoded by a nucleic acid sequence having at least 70% similarity to that of SEQ ID NO: 162, the Cas7 protein is encoded by a nucleic acid sequence having at least 70% similarity to that of SEQ ID NO: 164, and a Cas11 protein is encoded by the nucleic acid sequence having at least 70% similarity to that of SEQ ID NO: 166.

In certain embodiments, the Cas3 protein is encoded by the nucleic acid sequence of SEQ ID NO: 168, the Cas5 protein is encoded by the nucleic acid sequence of SEQ ID NO: 160, the Cas8 protein is encoded by the nucleic acid sequence of SEQ ID NO: 162, the Cas7 protein is encoded by the nucleic acid sequence of SEQ ID NO: 164, and the CasIl protein is encoded by the nucleic acid sequence of SEQ ID NO: 166. However, the invention is not limited to these exemplary sequences. Indeed, genetic sequences can vary between different strains, and this natural scope of allelic variation is included within the scope of the invention.

In certain embodiments, the Cas3 protein comprises the amino acid sequence of SEQ ID NO: 167, the Cas5 protein comprises the amino acid sequence of SEQ ID NO: 159, the Cas8 protein comprises the amino acid sequence of SEQ ID NO: 161, the Cas 7 protein comprises the amino acid sequence of SEQ ID NO: 163, and the Cas11 protein comprises the amino acid sequence of SEQ ID NO: 165.

However, the invention is not limited to these exemplary sequences. For example, in certain embodiments, the Cas3 protein comprises an amino acid sequence having at least 70% similarity to that of SEQ ID NO: 167, the Cas5 protein comprises an amino acid sequence having at least 70% similarity to that of SEQ ID NO: 159, the Cas8 protein comprises an amino acid sequence having at least 70% similarity to that of SEQ ID NO: 161, the Cas7 protein comprises an amino acid sequence having at least 70% similarity to that of SEQ ID NO: 163, and the Cas11 protein comprises an amino acid sequence of SEQ ID NO: 165.

In some embodiments, the Type I CRISPR-Cas system is a Type I-B system. Elements or sequences from any suitable type I-B CRISPR-Cas system may be used in the context of the disclosed methods. In some embodiments, the system comprises Cas11, Cas3, Cas5, Cas6, Cas7, and Cmx8.

In some embodiments, the Type I CRISPR-Cas system is a Type I-D system. Elements or sequences from any suitable type I-D CRISPR-Cas system may be used in the context of the disclosed methods. In some embodiments, the system comprises Cas11, Cas3, Cas5, Cas6, Cas7, and Cas10.

In some embodiments, the Type I-B or Type I-D CRISPR-Cas system is derived from the cyanobacteria Synechocystis (Syn). The primary strain of Synechocystis sp. is PCC6803. In some embodiments, the CRISPR-Cas system used in the context of the present disclosure is derived from the Type I system of Synechocystis sp. PCC6803, or variants thereof.

Synechocystis Type I CRISPR/Cas system proteins may comprise the wild-type amino acid sequence or variant having an amino acid sequence that is at least about 85% identical (e.g., about 86%, about 87%, about 88%, about 89%, about 90%, about 91%, about 92%, about 93%, about 94%, about 95%, about 96%, about 97%, about 98%, about 99%, or about 100%) to the amino acid sequence of any protein of the Synechocystis Type I CRISPR/Cas system proteins.

In certain embodiments, the Cas3 protein is encoded by a nucleic acid sequence having at least 70% similarity to that of 130, the Cas5 protein is encoded by a nucleic acid sequence having at least 70% similarity to that of SEQ ID NO: 126, the Cmx8 protein is encoded by a nucleic acid sequence having at least 70% similarity to that of SEQ ID NO: 122, the Cas6 protein is encoded by the nucleic acid sequence having at least 70% similarity to that of SEQ ID NO: 120, the Cas7 protein is encoded by a nucleic acid sequence having at least 70% similarity to that of SEQ ID NO: 123, and a Cas11 protein is encoded by the nucleic acid sequence having at least 70% similarity to that of SEQ ID NO: 128.

In certain embodiments, the Cas3 protein is encoded by the nucleic acid sequence of SEQ ID NO: 130, the Cas5 protein is encoded by the nucleic acid sequence of SEQ ID NO: 126, the Cmx8 protein is encoded by the nucleic acid sequence of SEQ ID NO: 122, the Cas6 protein is encoded by the nucleic acid sequence of SEQ ID NO: 120, the Cas7 protein is encoded by the nucleic acid sequence of SEQ ID NO: 123, and the Cas11 protein is encoded by the nucleic acid sequence of SEQ ID NO: 128. However, the invention is not limited to these exemplary sequences. Indeed, genetic sequences can vary between different strains, and this natural scope of allelic variation is included within the scope of the invention.

In certain embodiments, the Cas3 protein comprises the amino acid sequence of SEQ ID NO: 129, the Cas5 protein comprises the amino acid sequence of SEQ ID NO: 125, the Cmx8 protein comprises the amino acid sequence of SEQ ID NO: 121, the Cas6 protein comprises the amino acid sequence of SEQ ID NO: 119, the Cas7 protein comprises the amino acid sequence of SEQ ID NO: 124, and the Cas11 protein comprises the amino acid sequence of SEQ ID NO: 127.

However, the invention is not limited to these exemplary sequences. For example, in certain embodiments, the Cas3 protein comprises an amino acid sequence having at least 70% similarity to that of SEQ ID NO: 129, the Cas5 protein comprises an amino acid sequence having at least 70% similarity to that of SEQ ID NO: 125, the Cmx8 protein comprises an amino acid sequence having at least 70% similarity to that of SEQ ID NO: 121, the Cas6 protein comprises the amino acid sequence having at least 70% similarity to that of SEQ ID NO: 119, the Cas7 protein comprises an amino acid sequence having at least 70% similarity to that of SEQ ID NO: 124, and the CasIl protein comprises an amino acid sequence of SEQ ID NO: 127.

In certain embodiments, the Cas3 protein is encoded by a nucleic acid sequence having at least 70% similarity to that of 143, the Cas5 protein is encoded by a nucleic acid sequence having at least 70% similarity to that of SEQ ID NO: 138, the Cas6 protein is encoded by the nucleic acid sequence having at least 70% similarity to that of SEQ ID NO: 140, the Cas7 protein is encoded by a nucleic acid sequence having at least 70% similarity to that of SEQ ID NO: 136, the Cas 10 protein is encoded by a nucleic acid sequence having at least 70% similarity to that of SEQ ID NO: 134, and a Cas11 protein is encoded by the nucleic acid sequence having at least 70% similarity to that of SEQ ID NO: 141.

In certain embodiments, the Cas3 protein is encoded by the nucleic acid sequence of SEQ ID NO: 143, the Cas5 protein is encoded by the nucleic acid sequence of SEQ ID NO: 138, the Cas6 protein is encoded by the nucleic acid sequence of SEQ ID NO: 140, the Cas7 protein is encoded by the nucleic acid sequence of SEQ ID NO: 136, the Cas10 protein is encoded by the nucleic acid sequence of SEQ ID NO: 134, and the Cas11 protein is encoded by the nucleic acid sequence of SEQ ID NO: 141. However, the invention is not limited to these exemplary sequences. Indeed, genetic sequences can vary between different strains, and this natural scope of allelic variation is included within the scope of the invention.

In certain embodiments, the Cas3 protein comprises the amino acid sequence of SEQ ID NO: 144, the Cas5 protein comprises the amino acid sequence of SEQ ID NO: 137, the Cas6 protein comprises the amino acid sequence of SEQ ID NO: 139, the Cas7 protein comprises the amino acid sequence of SEQ ID NO: 135, the Cas10 protein comprises the amino acid sequence of SEQ ID NO: 133, and the Cas11 protein comprises the amino acid sequence of SEQ ID NO: 142.

However, the invention is not limited to these exemplary sequences. For example, in certain embodiments, the Cas3 protein comprises an amino acid sequence having at least 70% similarity to that of SEQ ID NO: 144, the Cas5 protein comprises an amino acid sequence having at least 70% similarity to that of SEQ ID NO: 137, the Cas6 protein comprises the amino acid sequence having at least 70% similarity to that of SEQ ID NO: 139, the Cas7 protein comprises an amino acid sequence having at least 70% similarity to that of SEQ ID NO: 135, the Cas10 protein comprises the amino acid sequence of SEQ ID NO: 133, and the Cas11 protein comprises an amino acid sequence of SEQ ID NO: 142.

Any of the proteins described herein may comprise one or more amino acid substitutions as compared to the corresponding wild-type protein. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH2 can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The one or more nucleic acids encoding the engineered CRISPR-Cas system may be any nucleic acid including DNA, RNA, or combinations thereof. In some embodiments, the one or more nucleic acids comprise one or more messenger RNAs, one or more vectors, or any combination thereof. For example, Cas11 may be encoded by a vector, whereas the two or more additional Cas proteins may be encoded by one or more messenger RNA.

In some embodiments, Cas11, Cas3, and the Cascade complex components are encoded by a single nucleic acid (e.g., a single vector). In some embodiments, Cas11, Cas3, and the Cascade complex components are encoded by different nucleic acids (e.g., multiple mRNAs or two or more vectors). In some embodiments, any combination of Cas11, Cas3, and the Cascade complex components are encoded on the same nucleic acid. For example, Cas11 and Cas3 may be encoded on the same vector, whereas the Cascade complex components may be encoded on a separate vector. Alternatively, Cas 11 may be encoded on a first vector, Cas3 may be encoded on a second vector, and the Cascade complex components may be encoded on a third vector.

In certain embodiments, engineering the system for use in eukaryotic cells may involve codon-optimization or other modification (e.g., to include an appropriate nuclear localization signal (NLS) or purification tag). It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons. Furthermore, in some embodiments, engineering the CRISPR-Cas system involves incorporating elements of the native CRISPR array into the disclosed system.

The system and the nucleic acid disclosed herein may comprise at least one guide RNA (gRNA). wherein each gRNA is configured to hybridize to a target nucleic acid sequence. The gRNA may be a crRNA or a crRNA/tracrRNA (e.g., single guide RNA, sgRNA) fusion. The terms “gRNA” and “guide RNA” refer to any nucleic acid comprising a sequence that determines the binding specificity of the CRISPR-Cas complex. In instances in which the system comprises two or more guide RNAs, each guide RNA may hybridize to a different target nucleic acid sequence.

The at least one gRNA may be encoded on the same or different nucleic acid as any of Cas11, Cas3, and the Cascade complex components. For example, a single vector may encode any or all of the at least one gRNA, Cas11, Cas3, and the Cascade complex components.

The terms “target DNA sequence,” “target nucleic acid,” “target sequence,” and “target site” are used interchangeably herein to refer to a polynucleotide (nucleic acid, gene, chromosome, genome, etc.) to which a guide sequence (e.g., a guide RNA) is designed to have complementarity, wherein hybridization between the target sequence and a guide sequence promotes the formation of a CRISPR/Cas complex, provided sufficient conditions for binding exist. The target sequence and guide sequence need not exhibit complete complementarity, provided that there is sufficient complementarity to cause hybridization and promote formation of a CRISPR complex. In some embodiments the system further comprises at least one target nucleic acid.

A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncomplementary strand” or “non-complementary strand.”

The target nucleic acid sequence may include a protospacer adjacent motif (PAM). A PAM can be 1, 2, 3, 4, 5, 6, 7, 8, 9, 10 or more nucleotides in length. In certain embodiments, a PAM is between 2-6 nucleotides in length. In some embodiments, the PAM is 3 nucleotides in length. The PAM may be “adjacent to” the target nucleic acid sequence in that it typically immediately precedes the target sequence. In some embodiments, the PAM is 5′ of the target site.

PAM sequences are often specific to the particular Cas endonuclease being used in the CRISPR/Cas complex and the species from which it was derived. For example, Type I-C CRISPR-Cas3 elements typically are active in a host cell genome which comprises a protospacer adjacent motif (PAM) comprising the nucleic acid sequence 5′-TTC-3′ or 5′-TTT-3′ located adjacent to the target genomic DNA sequence. PAM sequences and methods of determining PAM sequences for specific Cas proteins are known in the art. The gRNA or portion thereof that hybridizes to a target nucleic acid sequence (e.g., the guide sequence) may be between any length.

The guide sequence of the gRNA does not need to be completely complementary to the target site. In some embodiments, the guide sequence of the gRNA is at least 50%, 55%, 60%, 65%, 70%, 75%, 80%, 85%, 90%, 95%, 96%, 97%, 98%, 999%, or at least 100% complementary to the target site. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target site (e.g., the last 5, 6, 7, 8, 9, or 10 nucleotides of the 3′ end of the target site). “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence.

To facilitate gRNA design, many computational tools have been developed (See Prykhozbij et al. (PLOS ONE, 10(3): (2015)); Zhu et al. (PLOS ONE, 9(9) (2014)); Xiao et al. (Bioinformatics. Jan 21 (2014)): Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s): including but not limited to, Genscript Interactive CRISPR gRNA Design Tool, WU-CRISPR, and Broad Institute GPP sgRNA Designer.

In addition to the guide sequence, in some embodiments, a gRNA may also comprise a scaffold sequence (e.g., tracrRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, at least one gRNA is within a crRNA array. A crRNA array comprises multiple guide RNAs (sgRNA) derived from the fusion of CRISPR RNA (crRNA) and trans-activating crRNA (tracrRNA) expressed a single transcript, which after processing by a nuclease are cleaved into separate gRNAs. The crRNA array may contain multiple repeats separated by unique spacers. For example, an engineered crRNA array may comprise contains two repeats and one spacer, or three repeats and two identical spacers. An exemplary crRNA array-repeat amino acid sequence may comprise SEQ ID NO: 114, SEQ ID NO: 131, SEQ ID NO: 145, SEQ ID NO: 157 or SEQ ID NO: 169.

One or all of the at least one gRNAs may be a non-naturally occurring gRNA.

In some embodiments, the system comprises two or more engineered CRISPR-Cas systems or one or more nucleic acids encoding two or more engineered (CRISPR-Cas) systems. Desirably, the two or more engineered CRISPR-Cas systems are derived from different subtypes of Type I CRISPR-Cas systems. Desirably, the two or more engineered CRISPR-Cas systems are orthogonal, which means that each CRISPR-Cas system only functions with its own cognate components (e.g., Cas proteins, PAM sequences, and crRNA (gRNA, spacer, and repeat sequences)).

In some embodiments, the two or more engineered CRISPR-Cas systems comprise two Type I CRISPR-Cas systems selected from the group consisting of a Type I-B CRISPR-Cas system, a Type I-C CRISPR-Cas system, and a Type I-D CRISPR-Cas system. The two or more engineered CRISPR-Cas systems may be selected from a N. lactamica Type I-C derived system, a Synechocystis Type I-D derived system, a Synechocystis Type I-B system, a Bacillus Type I-C derived system and a Desulfovibrio, Type I-C derived system.

In some embodiments, the system is a cell-free system.

The vector(s) comprising the nucleic acid sequences encoding the at least one gRNA, Cas11, Cas3, and the two or more additional Cas proteins for the system(s) can be introduced into a cell that is capable of expressing the polypeptide encoded thereby, including any suitable prokaryotic or eukaryotic cell.

Conventional viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present system and/or components to the cells, tissues and/or a subject. Viral vectors include, for example, retroviral, lentiviral, adenoviral, adeno-associated and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71.

Drug selection strategies may be adopted for positively selecting for cells comprising the nucleic acid sequences encoding the present system or components thereof.

The present disclosure also provides for DNA segments encoding the proteins and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

To construct cells that express the present system, expression vectors for stable or transient expression of the present system may be constructed via conventional methods and introduced into cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL., Cold Spring Harbor Laboratory, Cold Spring Harbor Laboratory Press, Cold Spring Harbor, N.Y., incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), Hl (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells: enhancer/promoter sequences from the immediate early gene of human CMV for high levels of transcription: transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication: internal ribosome binding sites (IRESes), versatile multiple cloning sites; T7 and SP6 RNA promoters for in vitro transcription of sense and antisense RNA: a “suicide switch” or “suicide gene” which when triggered causes cells carrying the vector to die (e.g., HSV thymidine kinase, an inducible caspase such as iCasp9), and reporter gene for assessing expression.

When introduced into a cell, the vectors may be maintained as an autonomously replicating sequence or extrachromosomal element or may be integrated into host DNA.

The present system or components thereof may be delivered to a cell by any suitable means. In certain embodiments, the system is delivered in vivo. In other embodiments, the system is delivered to isolated/cultured cells in vitro or ex vivo to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan 1;459(1-2):70-83), incorporated herein by reference.

In other embodiments, various components of the system may be introduced into a host cell as a ribonucleoprotein (RNP) complex. The term “ribonucleoprotein complex,” as used herein, refers to a complex of ribonucleic acid and RNA-binding protein(s). In the context of CRISPR-Cas systems, an RNP complex typically comprises Cas protein(s) (e.g., Cas5, Cas7, and Cas8) in complex with a gRNA. RNPs may be assembled in vitro and can be delivered directly to cells using standard electroporation, cationic lipids, gold nanoparticles, or other transfection techniques (see, e.g., Kim et al., Genome Res., 24: 1012-1019 (2014): Zuris et al., Nat. Biotechnol., 33: 73-80 (2015); and Mout et al., ACS Nano., 11: 2452-2458 (2017)).

As such, the disclosure provides an isolated cell comprising the system, the vector(s), nucleic acid(s), or system disclosed herein. The disclosure also provides populations of cells comprising the present systems.

Preferred cells are those that can be easily and reliably grown, have reasonably fast growth rates, have well characterized expression systems, and can be transformed or transfected easily and efficiently, including both eukaryotic and prokaryotic cells. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993), incorporated herein by reference.

Desirably, the cell is a mammalian cell, and in some embodiments, the cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-I (ATCC No. CRL1650) and COS-7 cell lines (ATCC No. CRL1651), as well as the CV-I cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines.

Methods for selecting suitable mammalian cells and methods for transformation, culture,

amplification, screening, and purification of cells are known in the art.

The system may further comprise components in addition to those listed, including, but not limited to: sequence tags, protein markers or marker proteins, spacers, capture sequences, and the like.

### 3. Methods of Altering a Target Nucleic Acid

The disclosure also provides a method of altering a target nucleic acid sequence. The phrase “altering a DNA sequence,” as used herein, refers to modifying at least one physical feature of a DNA sequence of interest. DNA alterations include, for example, single or double strand DNA breaks, deletion, or insertion of one or more nucleotides, and other modifications that affect the structural integrity or nucleotide sequence of the DNA sequence.

The methods comprise contacting a target nucleic acid sequence with a system disclosed herein or a composition comprising the system.

In one embodiment, the method introduces a single strand or double strand break in the target DNA sequence. In this respect, the disclosed systems may direct cleavage of one or both strands of a target DNA sequence, such as within the target genomic DNA sequence and/or within the complement of the target sequence.

In some embodiments, altering a DNA sequence comprises a deletion. The deletion may be upstream or downstream of the PAM binding side, so called unidirectional deletions. The deletion may encompass sequences on either side of the PAM binding site, a bidirectional deletion. In some embodiments, the system introduces unidirectional DNA deletions. In some embodiments, the system introduces bidirectional DNA deletions. In some embodiments, the system introduces a deletion without prominent off-target activity.

The deletion of the DNA sequence may be of any size. For example, in some embodiments the deletion of the DNA sequence comprises from about 500 nucleotides to about 100,000 nucleotides (e.g., about 1,000, 5,000, 10,000, or 50,000 nucleotides, or a range defined by any two of the foregoing values). In other embodiments, the deletion of the DNA sequence comprises from about 5,000 nucleotides to about 20,000 nucleotides (e.g., about 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, or 19,500 nucleotides, or a range defined by any two of the foregoing values).

In some embodiments, the contacting a target nucleic acid sequence comprises introducing the system into the cell. As described above the system may be introduced into eukaryotic or prokaryotic cells by methods known in the art. In some embodiments, the cell is a mammalian cell. In some embodiments, the cell is a human cell.

In some embodiments, introducing the system into a cell comprises administering the system to a subject. In some embodiments, the subject is human. The administer may comprise in vivo administration. In alternative embodiments, a vector is contacted with a cell in vitro or ex vivo and the treated cell, containing the system, is transplanted into a subject.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product.” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

The disclosed method may alter a target DNA sequence in a host cell so as to modulate expression of the target DNA sequence, e.g., expression of the target DNA sequence is increased, decreased, or completely eliminated (e.g., via deletion of a gene). In one embodiment, the disclosed system cleaves a target DNA sequence of the host cell to produce double strand DNA breaks. The double strand breaks can be repaired by the host cell by either non-homologous end joining (NHEJ) or homologous recombination. In NHEJ, the double-strand breaks are repaired by direct ligation of the break ends to one another. In homologous recombination repair, a donor nucleic acid molecule comprising a second DNA sequence with homology to the cleaved target DNA sequence is used as a template for repair of the cleaved target DNA sequence, resulting in the transfer of genetic information from the donor nucleic acid molecule to the target DNA. As a result, new nucleic acid material is inserted/copied into the DNA break site. The modifications of the target sequence due to NHEJ and/or homologous recombination repair may lead to, for example, gene correction, gene replacement, gene tagging, transgene insertion, nucleotide deletion, gene disruption, gene mutation, gene knock-down, etc.

In some embodiments, the systems and methods described herein may be used to correct one or more defects or mutations in a gene (referred to as “gene correction”). In such cases, the target sequence encodes a defective version of a gene, and the disclose system further comprises a donor nucleic acid molecule which encodes a wild-type or corrected version of the gene. Thus, in other words, the target sequence is a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, a-1 antitrypsin, cystic fibrosis transmembrane conductance regulator (CFTR), B-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB). neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping. SNPs, and Microarray Data, Nature Education 1(1): 192 (2008): Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects.

In another embodiment, the method of altering a target sequence can be used to delete nucleic acids from a target sequence in a host cell by cleaving the target sequence and allowing the bost cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

The disclosure further provides kits containing one or more reagents or other components useful, necessary, or sufficient for practicing any of the methods described herein. For example, kits may include CRISPR reagents (Cas proteins, guide RNAs, vectors, compositions, etc.), transfection or administration reagents, negative and positive control samples (e.g., cells, template DNA), cells, containers housing one or more components (e.g., microcentrifuge tubes, boxes), detectable labels, detection and analysis instruments, software, instructions, and the like.

Any element of any suitable CRISPR/Cas gene editing system known in the art can be employed in the systems and methods described herein, as appropriate. CRISPR/Cas gene editing technology is described in detail in, for example, U.S. Pat. Nos. 8,546,553, 8,697,359; 8,771,945; 8,795,965; 8,865,406; 8,871,445; 8,889,356; 8,889,418; 8,895,308; 8,9066,616; 8,932,814; 8,945,839; 8,993,233; 8,999,641: 9,115,348; 9,149,049; 9,493,844; 9,567,603: 9,637,739; 9,663,782; 9,404,098; 9,885,026; 9,951,342; 10,087,431: 10,227,610; 10,266,850; 10,601,748; 10,604,771; and 10,760,064; and U.S. Patent Application Publication Nos. US2010/0076057; US2014/0113376; US2015/0050699; US2015/0031134; US2014/0357530; US2014/0349400; US2014/0315985; US2014/0310830; US2014/0310828; US2014/0309487; US2014/0294773; US2014/0287938; US2014/0273230; US2014/0242699: US2014/0242664; US2014/0212869; US2014/0201857; US2014/0199767; US2014/0189896; US2014/0186919; US2014/0186843; and US2014/0179770, each incorporated herein by reference.

The following examples further illustrate the invention but should not be construed as in any way limiting its scope.

### EXAMPLES

### Materials and Methods

Informatic prediction of the protospacer adjacent motif (PAM) The native CRISPR array of Neisseria lactamica ATCC 23970 strain contains 30 spacers. Natural target sequences were bioinformatically searched for all the spacers using CRISPRTarget, allowing for up to 1 nt mismatch in the spacer-target complementarity. 28 unique targets were identified. For these targets, the spacer-matched sequences were extracted together with 10 nt flanking regions on both 5′- and 3′-sides and aligned using WebLogo. A conserved 5′ flanking TTC motif was deduced.

CRISPR DNA interference assay in E. coli The Nla I-C cas3, cascade, and erRNA plasmids were co-transformed into BL21-AI cells. The resulting strain was made competent using the Mix and Go kit (Zymo), and then transformed with a target-containing pCDFI plasmid, resulting in an E. coli strain harboring all four plasmids. For interference assay, a single colony of this final E. coli strain was inoculated into 2 mL LB culture with four antibiotics (kanamycin, carbenicillin, spectinomycin, and chloramphenicol, selective for all plasmids) and grown to OD600 0.3 at 37° C. The culture was then pelleted, resuspended in LB with three antibiotics (Kan, Carb, and Cm), and then split in two halves. One was induced with 0.2% L-arabinose and 1 mM IPTG. Both the induced and un-induced cultures were grown for an additional 3 hours at 37° C. Cultures were then serially 10-fold diluted and plated onto LB plates containing quadruple vs. triple antibiotics (lacking spectinomycin). The ratio of colony forming units between the two plates represents the efficiency of CRISPR interference.

Plasmid transfection The HAP1 reporter cells were transfected using Lipofectamine 3000 reagent (ThermoFisher) according to the manufacturer's instructions. The reporter cells were seeded one day before transfection at 1×10⁵ cells per well of a 24-well plate. For each transfection, 500 ng plasmid was used (For Nla I-C system: 45 ng of Cas3, 22.5 ng of Cas5, 67.5 ng of Cas7, 270 ng of Cas8, 45 ng of Cas11 and 50 ng of CRISPR; For Bha I-C system: 67.5 ng of Csd2, 22.5 ng of Cas5, 270 ng of Csd1, 45 ng of Cas3, 45 ng of Cas11 and 50 ng of CRISPR: For Dvu 1-C system: 90 ng of Cas5, 90 ng of Cas7, 90 ng of Cas8, 90 ng of Cas3, 90 ng of Cas11 and 50 ng of CRISPR; For Syn I-D system: 180 ng of Cas5, 76.5 ng of Cas6, 15 ng of Cas7, 76.5 ng of Cas3, 76.5 ng of Cas10, 22.5 ng of Cas11 and 50 ng of CRISPR; For Syn I-B system, 75 ng of Cas3, 75 ng of Cas5, 75 ng of Cas6, 75 ng of Cas7, 75 ng of Cmx8, 75 ng of Cas11 and 50 ng of CRISPR) along with 1 μL of P3000 reagent and 1.5 μL of Lipofectamine 3000 reagent. Cells were analyzed using flow cytometry 4-5 days after transfection.

Protein purification of Nla-NLS-Cascade Two methods were designed to purify Nla-Cascade complex. The first method used MBP affinity purification with an MBP tagged Cas5 protein followed by size exclusion chromatography. The second method used Ni affinity purification with a His tagged Cas7 protein followed by size exclusion chromatography.

Method 1: The two plasmids expressing 6xHis-MBP-cas5-cas8c-cas7-NLS and CRISPR were co-transformed into BL21(DE3) cells. The resulting strain was then inoculated into 10 mL of LB with 50 μg/mL of kanamycin and 20 μg/ml of chloramphenicol, and grown overnight at 37° C. This overnight culture was then used to inoculate a 1 L of LB containing 50 μg/mL. kanamycin, 20 μg/mL chloramphenicol and 0.2% glucose. The big culture was cooled to 18° C. when it reached OD600 ˜0.6 and induced with 1 mM IPTG for 18 hr at 18° C. Cells were then pelleted and resuspended in 20 mM HEPES pH 7.5 and 500 mM NaCl, and then lysed with sonication. MBP-tagged protein was bound to amylose beads (NEB) and eluted with buffer containing 20 mM HEPES pH7.5, 500 mM NaCl, and 10 mM maltose. Eluted proteins were incubated with TEV protease overnight to cleave off the His-MBP tag, concentrated, and then further purified on a sephacryl S300 column. Cascade containing fractions were pooled, dialyzed into 20 mM HEPES pH7.5, 150 mM NaCl, concentrated, filter sterilized, aliquoted, and frozen in liquid nitrogen.

Method 2: The two plasmids expressing cas5-cas8c-cas7-NLS-6xHis and CRISPR were co-transformed into BL21(DE3) cells. The resulting strain was then inoculated into 10 mL of LB with 50 μg/mL of kanamycin and 20 μg/ml of chloramphenicol, and grown overnight at 37° C. This overnight culture was then used to inoculate a 1 L LB containing 50 μg/mL kanamycin, 20 μg/mL chloramphenicol. The big culture was cooled to 18° C. when it reached OD600 ˜0.6 and induced with 1 mM IPTG for 18 hr at 18° C. Cells were then pelleted and resuspended in 30 mM HEPES pH 7.5, 500 mM NaCl and 0.5 mM TCEP, and then lysed with sonication. His-tagged protein was bound to Ni-NTA resin (Qiagen) and eluted with buffer containing 30 mM HEPES pH7.5, 500 mM NaCl, and 300 mM imidazole. Eluted proteins were concentrated, and then further purified on a sephacryl S300 column using 30 mM HEPES pH 7.5, 150 mM NaCl and 0.5 mM DTT as elution buffer. Cascade containing fractions were pooled, concentrated, filter sterilized, aliquoted, and frozen in liquid nitrogen.

Purification of NlaCas3 The plasmid expressing Nla cas3-NLS-6xHis was transformed into BL21(DE3) cells. The resulting strain was then inoculated into 10 ml of LB with 50 μg/mL of kanamycin and grown overnight at 37° C. This overnight culture was then used to inoculate a 1 L LB containing 50 μg/mL kanamycin. The big culture was cooled to 18° C. when it reached OD600 ˜0.6 and induced with 1 mM IPTG for 18 hr at 18° C. Cells were then pelleted and resuspended in 30 mM HEPES pH 7.5, 500 mM NaCl and 0.5 mM TCEP, and then lysed with sonication. His-tagged protein was bound to Ni-NTA resin (Qiagen) and eluted with buffer containing 30 mM HEPES pH7.5, 500 mM NaCl, and 300 mM imidazole. Eluted proteins were concentrated, and then further purified on a sepbacryl S300 column using 30 mM HEPES pH 7.5, 150 mM NaCl and 0.5 mM DTT as elution buffer. Cas3 containing fractions were pooled, concentrated, filter sterilized, aliquoted, and frozen in liquid nitrogen.

RNP electroporation All cells were electroporated using Neon Transfection system (ThermoFisher) according to the manufacturer's instruction. Briefly, cells were individualized with TrypLE Express (Gibco), washed once with culturing media and resuspended in Neon buffer R to a concentration of 2×10⁷ cells/mL. 36 pmol of NLS-NlaCascade with or without 50 pmol of NLS-NlaCas3 protein were mixed with approximately 10⁵ cells in buffer R in a total volume of 10 μL. Each mixture was then electroporated with a 10 μL Neon tip (HAP1: 1575V 10 ms 3 pulses; hESC: 1100V 20 ms 2 pulses; 293T: 1150V 20 ms 2 pulses; Hela: 1005V 35 ms 2 pulses.) and plated in 24-well tissue culture plates containing 500 μL appropriate culturing media. Cells were analyzed 4-5 days after electroporation.

Generation of mRNA through in vitro transcription mRNAs used for electroporation were generated by in vitro transcription using mMessage mMachine T7 Ultra kit (ThermoFisher) following the manufacture's protocol. Templates for in vitro transcription were generated via PCR amplification using human codon optimized Nla cas genes as templates.

mRNA delivery into HAP1 reporter cells by electroporation The HAP1 reporter cells were electroporated using Neon Transfection system (ThermoFisher) according to the manufacturer's instruction. Briefly, the cells were individualized with TrypLE Express (Gibco), washed once with IMDM, 10% FBS and resuspended in Neon buffer R to a concentration of 4×10⁷ cells/mL. Approximately 2×10⁵ cells were mixed with 50 ng of Nla cas3 mRNA, 120 ng of Nla cas5 mRNA, 120 ng of Nla cas7 mRNA, 140 ng of Nla cas8 mRNA, 120 ng of Nla cas11 mRNA and 200 ng of CRISPR plasmid (or 2 μg of CRISPR RNA) in buffer R in a total volume of 10 μL. Each mixture was then electroporated with a 10 μL Neon tip (1575V, 10 ms, 3 pulses) and plated in 24-well tissue culture plates containing 500 μL IMDM, 10% FBS. Cells were analyzed by flow cytometry 4-5 days after electroporation.

DNA lesion analysis by long-range PCR and cloning Genomic DNAs of edited cells were isolated using Gentra Puregene Cell Kit (Qiagen) per manufacturer's instruction. Long-range PCRs were done using Q5 DNA Polymerase (NEB). Products were resolved on 1% agarose gel stained by SYBR Safe (Invitrogen) and visualized with ChemiDoc MP imager (Biorad).

To define lesion junctions, the PCR reactions were purified using QIAquick PCR Purification Kit (Qiagen) and cloned into pCR-BluntII-TOPO vector (Invitrogen). Colony PCR with M13 forward and reverse primers were carried out from the resulting colonies. Positive clones were randomly selected for Sanger sequencing (Eurofin). Deletion junctions were identified by aligning the sequencing results to the reference WT sequence using Snapgene.

Flow Cytometry and Analysis Cells were individualized with TrypLE Express (Gibco) 4-5 days after RNP electroporation, or plasmid transfection, resuspended in IMDM+ 10% FBS (for HAP1 cells) or DMEM/F12+10% FBS (for hES cells), and then kept on ice until analysis. Cells were analyzed on LSR Fortessa (BD) using a 488 nm laser for EGFP and a 561 nm laser for tdTomato. Flow cytometry data were analyzed with FlowJo.

6-TG Selection Assay HAP1 cells were individualized by TrypLE Express 2 days after RNP electroporation and then seeded in 6-well plate at a density of 200 cells/well. Two days after cell seeding, 6-TG (6-Thioguanine, Sigma) were added to each well at a final concentration of 15 μM. Media containing 6-TG was changed every 2 days. 6 days after 6-TG treatment, cells were fixed with ice-cold 90% methanol for 30 min, washed once with 1×PBS, stained with 0.5% crystal violet at RT for 5 min and destained with water. The plates are then air-dried at RT overnight and imaged by ChemiDoc MP imaging system (BioRad). The surviving colony numbers were then counted by OpenCFU(Geissmann, 2013).

Western Blot Cells were lysed directly on plate using 100 μl lysis buffer (45 μl of PBS, 50 μl of 2×Laemmli buffer, 5 μl of 1 M DTT, 0.4 μl benzonase) per well of a 24-well plate 2 days after transfection. The cell lysate was then incubated at 95° C. for 5 min, separated on a 12% SDS polyacrylamide gel and transferred to a PVDF membrane. The membrane was then blocked in blocking buffer (3% non-fat milk in TBST) at RT for 40 min and probed with appropriate primary antibodies followed by HRP conjugated secondary antibodies. After incubation with ECL Western Blot Detection Reagent (GE Healthcare), the membrane was imaged using ChemiDoc MP Imaging system (BioRad).

### Example 1

### A novel compact CRISPR-Cas3 from N. lactamica confers robust CRISPR Interference In Vivo

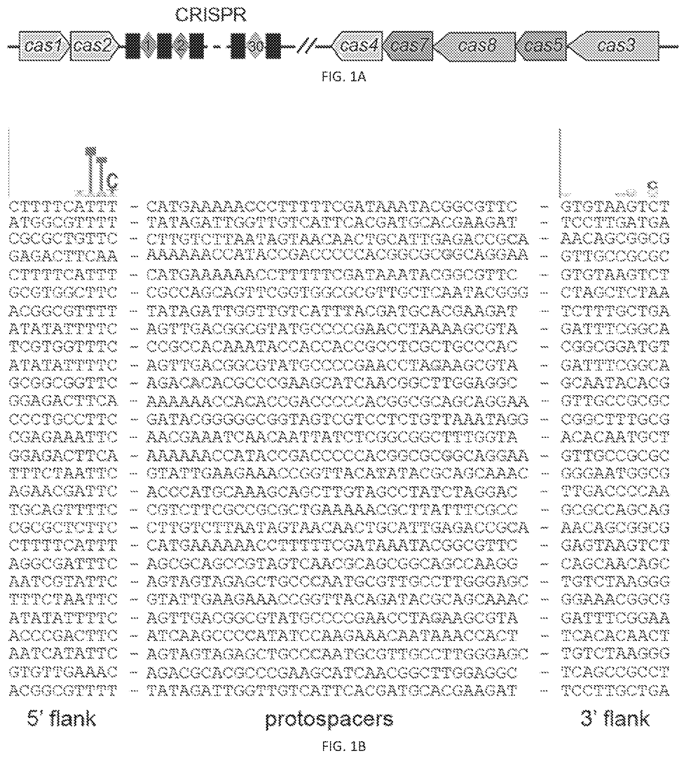

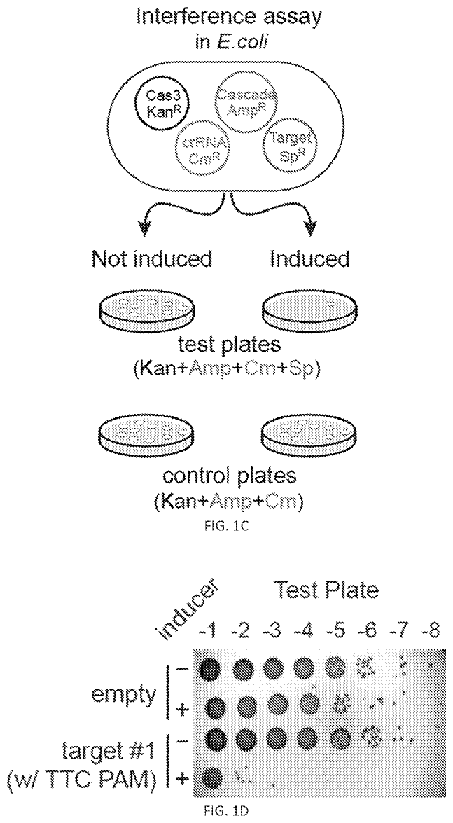

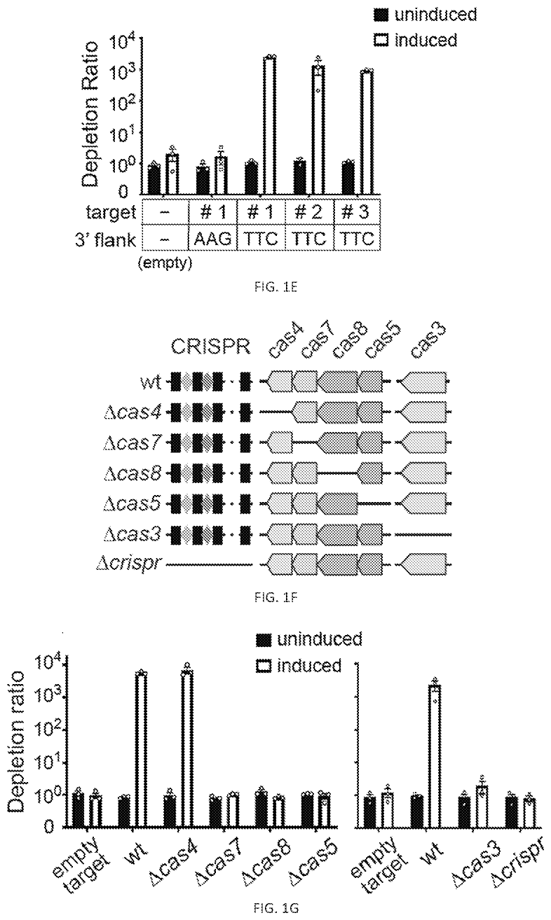

In search for a novel and compact Type I CRISPR, the genomes of Neisseria spp. were examined and a previously uncharacterized Type I-C CRISPR-Cas from N. lactamica strain ATCC 23970 was identified. It consists of a CRISPR array and seven cas genes, including the spacer acquisition genes cas1, cas2, and cas4, the nuclease-helicase gene cas3, and the set of genes (cas5, cas8 and cas7) encoding protein subunits of Cascade (FIG. 1A). The native CRISPR array contains thirty spacers 34-35 bp in length, sandwiched between 32-bp repeats. The PAM sequences were defined informatically, by first looking for potential natural targets of all the natural spacers using CRISPRTarget, allowing for up to 1 nt mismatch in the spacer-target complementarity. A total of 28 unique targets were found. When these protospacers sequences were aligned, along with their 10 bp flanking regions immediately upstream and downstream, a strong 5′-TTC PAM motif was revealed (FIG. 1B).

The functionality of this Nla I-C CRISPR system was tested by conducting a plasmid interference assay using E. coli as a surrogate host (FIG. (C). The cas5-8-7-4 operon was cloned into pBAD vector under the control of an arabinose inducible promoter, cas3 into pET28b under a T7 promoter, the native CRISPR into pACYC under a T7 promoter and the potential target sequences into pCDF1. BL21-AI derivative strains harboring all four plasmids were built and the induced culture was plated on quadruple antibiotics LB plates to track cell survival. Induction of crispr-cas expression led to ˜1,000-fold reduction in colony counts, if the target plasmid contained a 5′-TTC PAM followed by sequence complementary to any of the first three native CRISPR spacers, but not when an empty target plasmid was used as negative control (FIGS. 1D-E). This result indicated a robust plasmid interference phenotype in vivo. A control target for spacer 1 with a 5′-AAG motif failed to elicit interference, suggesting that a functional PAM is a prerequisite for Nla I-C system to mount successful CRISPR defense (FIG. 1E).

To determine the other components facilitating the interference, a series of deletion mutants, each lacking a different crispr-cas gene, were analyzed (FIG. 1F). Interference was completely abrogated by internal deletion of cas7, cas8, or cas5, but not the cas4 gene, from the pCascade plasmid; strains lacking cas3 or the CRISPR array were also defective for interference (FIG. 1G). Collectively, the results showed that DNA targeting by Nla I-C CRISPR utilized a matching spacer-target pair, a functional PAM, cas3 and all Cascade subunit genes, whereas the putative spacer acquisition genes cas1, cas2 and cas4 were dispensable.

### Example 2

### NlaCascade-Cas3 RNP Achieves High-Efficiency Multiplexed Genome Engineering in Human Cells

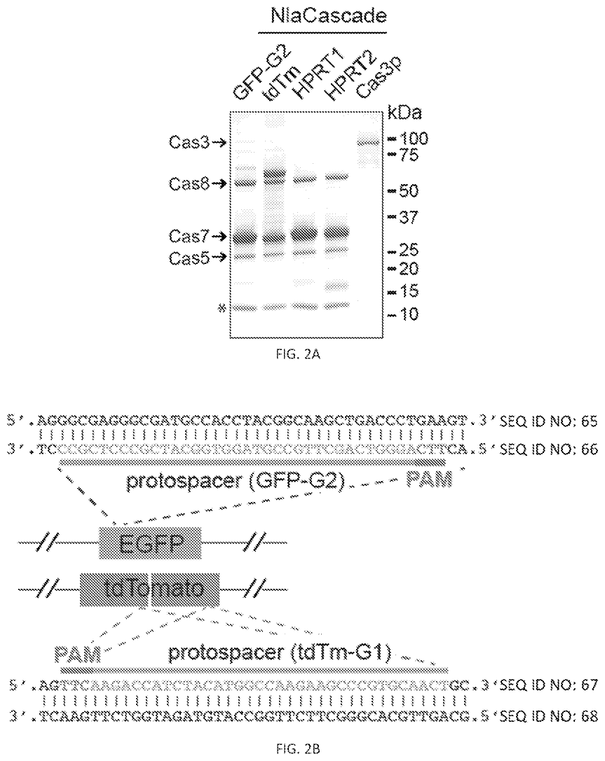

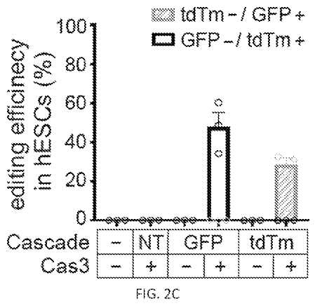

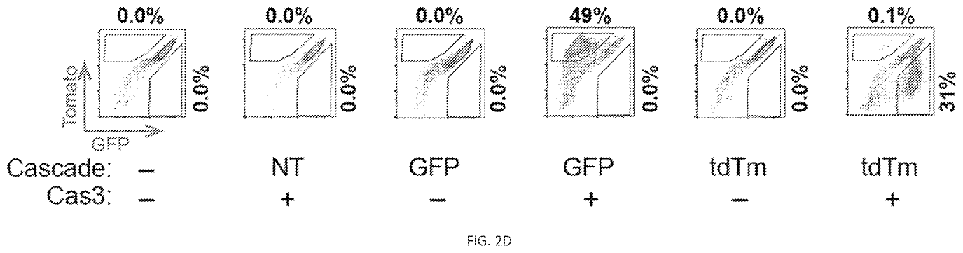

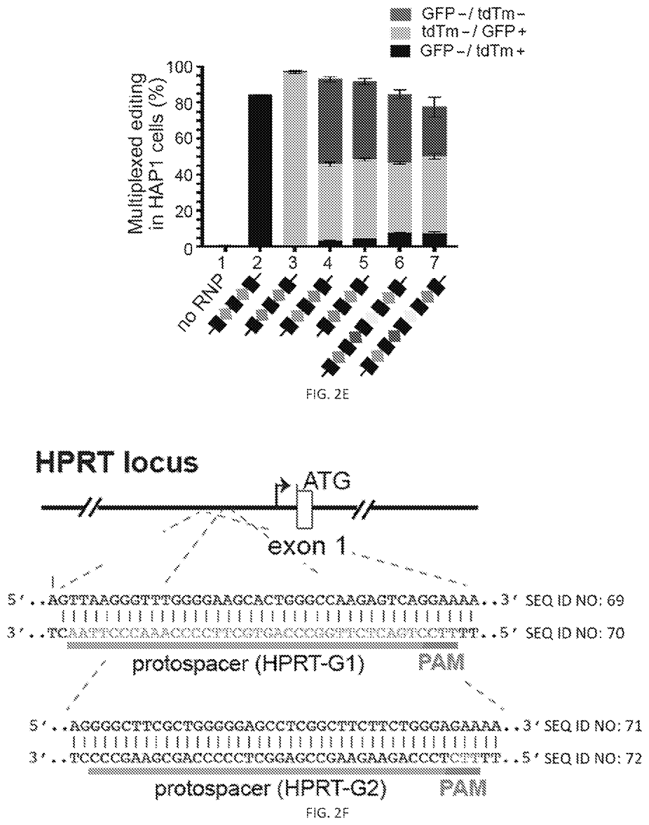

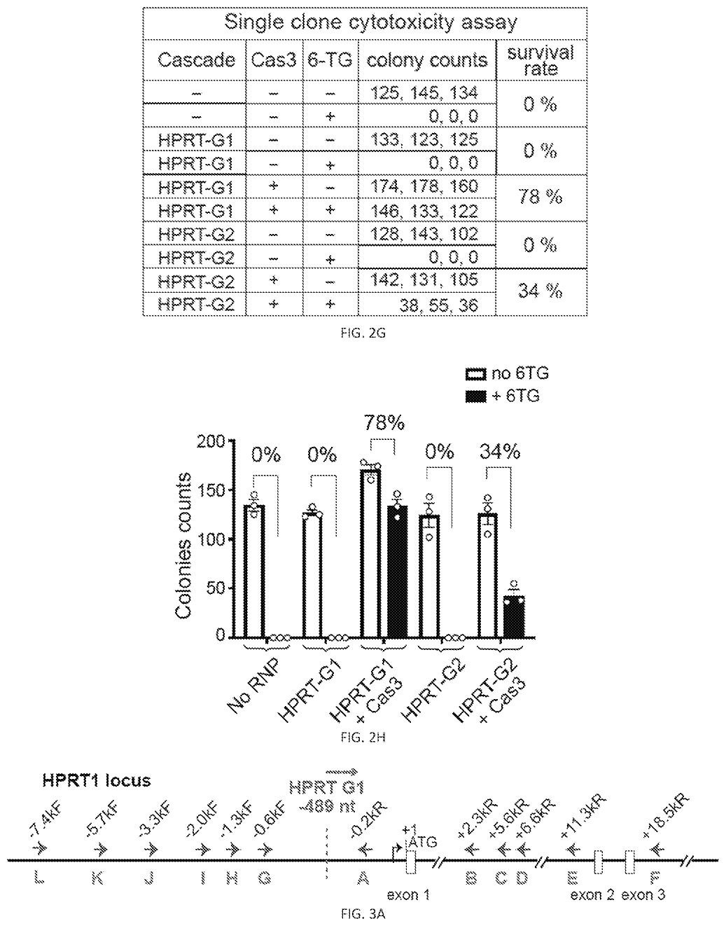

RNP-based genome editing was tested by purifying recombinant Cas3 and Cascade separately from E. coli (FIG. 2A), delivering them into various human cell lines via electroporation, and monitoring genome editing efficiency by flow cytometry. Initial editing experiments were carried out in a human embryonic stem cell (hESC) dual reporter line, with two CRISPR guides designed to target 5′-TTC-flanked sites in the EGFP or tdTomato (tdTm) genes respectively (FIG. 2B). The corresponding Cascade complexes containing nuclear localization signal (NLS) sequences on the C-termini of all Cas7 subunits were purified via nickel affinity pulldown and size exclusion chromatography (SEC), and then tested with or without purified NLS-Cas3. Roughly 50% and 30% editing rates were observed for EGFP and tdTm, respectively, when the cognate Cascade was used in conjunction with Cas3 (FIGS. 2C-D). Negative controls lacking Cas3, or containing a Cascade targeting either the other non-corresponding reporter gene or an endogenous genomic locus (non-targeting, NT) all failed to produce a signal above the untreated background (FIGS. 2C-D). The 30-50% editing efficiency obtained in bESC was quite impressive, given that prior work utilizing T. fusca Type I-E RNP only gave up to 13% editing in the same reporter cell.

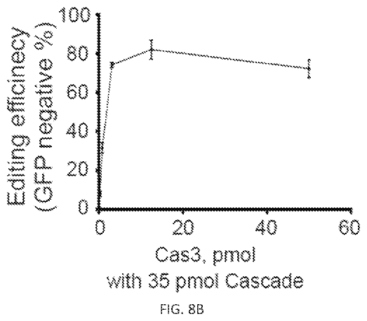

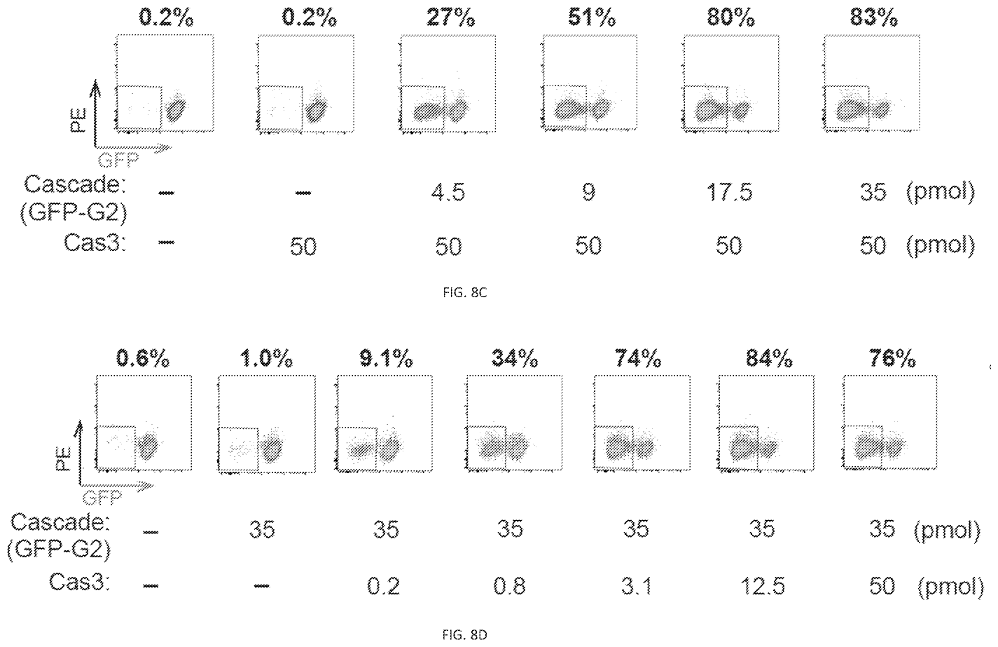

Parallel experiments were performed in a HAP1 reporter cell line using the same EGFP-targeting Cascade, and dose-dependent editing of up to 83% was obtained. As the amount of Cascade used went up from 4.5 to 35 pmol, editing efficiency gradually increased from 27% to 83% (FIGS. 8A and 8C). In contrast, for Cas3 titration editing jumped to and plateaued at ˜76% with as little as 3 pmol Cas3 delivered (FIGS. 8B and 8D). These results implied that genome editing with the current Nla CRISPR-Cas3 RNP platform was limited by the assembly or target searching activity of Cascade, but not the DNA degradation activity of Cas3.

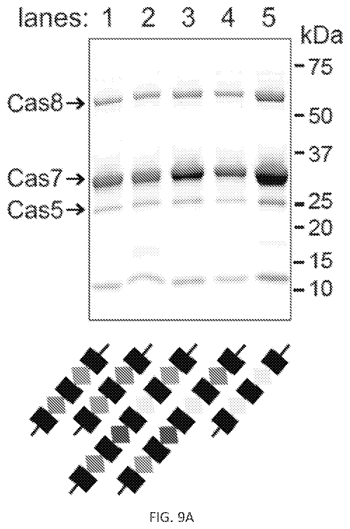

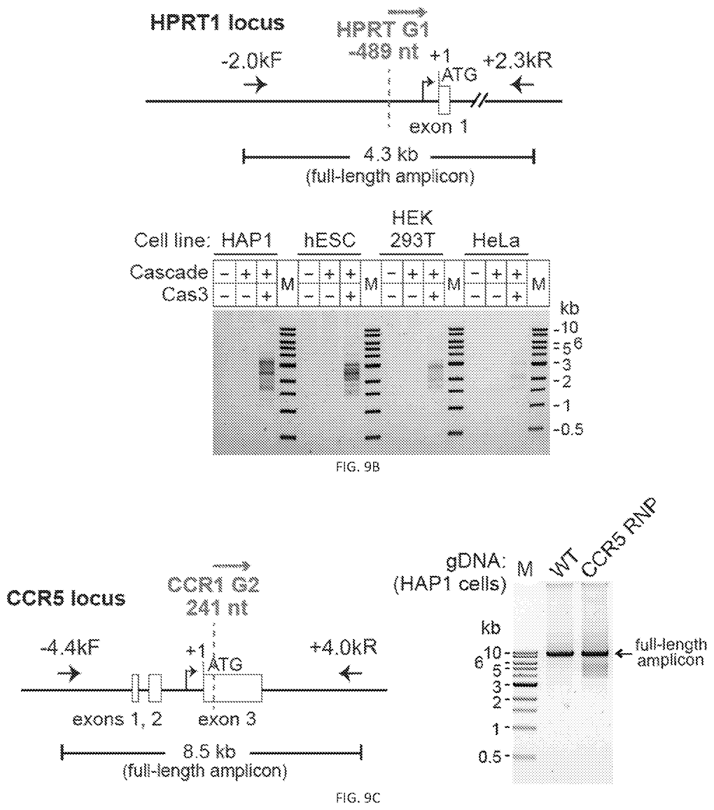

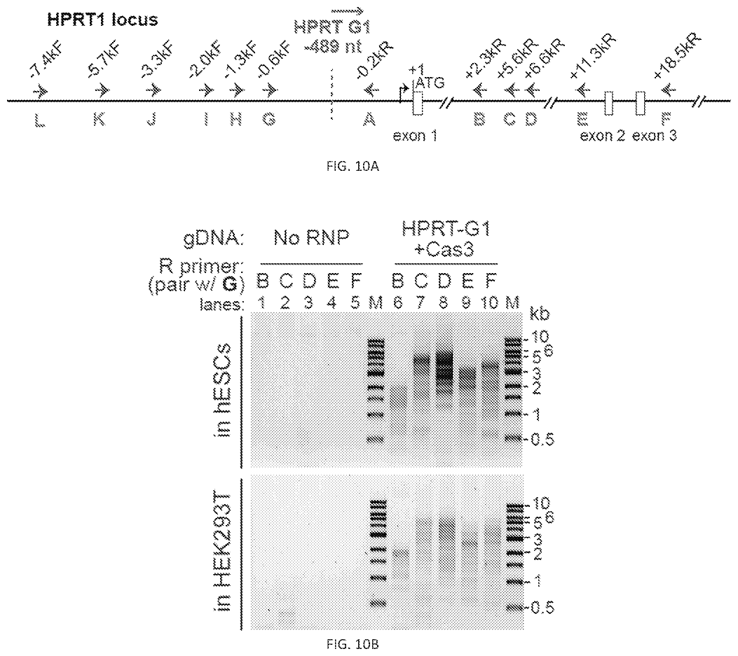

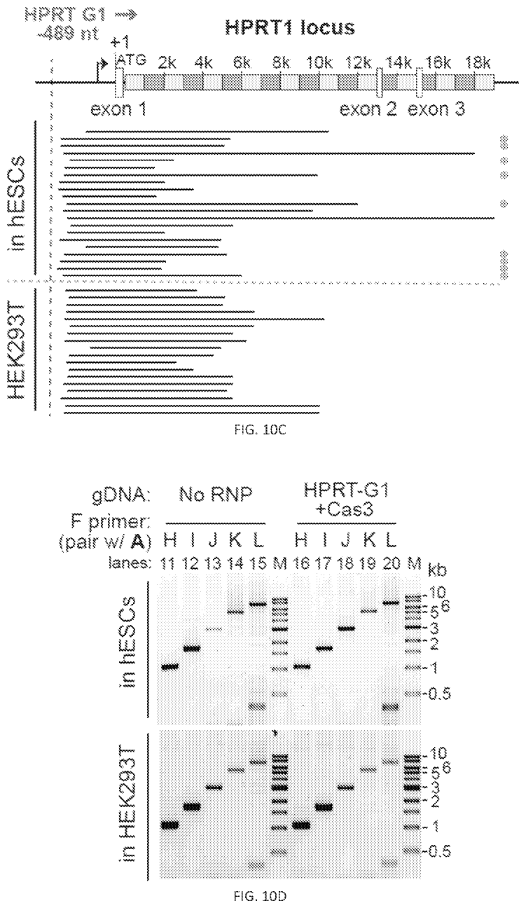

The CRISPR array of a Type I system is transcribed into a multi-unit primary transcript, which is then processed into individual mature crRNAs loaded in Cascade. The multi-spacer CRISPR cassette therefore offers a unique opportunity to co-express numerous guide RNAs and purify a collection of corresponding Cascade RNPs at once from E. coli. To explore this, two versions of the CRISPR in R-S-R-S-R configuration were created, each contained three repeats and two distinct intervening spacers at different relative positions (FIG. 2E, samples 4-5). When each resulting Cascade prep was electroporated with Cas3 into HAP1 reporter cell, concurrent disruption of both EGFP and tdTm fluorescence was observed in majority of the cells (FIG. 2E, 47% and 43% for the two arrays, respectively, and FIG. 9A), indicative of efficient multiplexed editing. Importantly, flow cytometry showed that multiplexing indeed occurred in individual human cell, not just on a population level. As controls, Cascade RNPs purified using arrays with two identical spacers targeting one reporter led to >85% editing of just the cognate fluorescent gene but not the other non-cognate one (FIG. 2E, samples 2-3).

To demonstrate facile programmability and broad applicability, the NlaCRISPR-Cas3 RNP was applied to target various endogenous genes in different human cell lines. The HPRT1 locus of the near-haploid HAP1 cells was used because its editing rate can be readily assessed using a single clone cytotoxicity assay measuring resistance to 6-thioguanine (6-TG) mediated cell killing. Cascade RNP targeting the promoter region 489 bp or 274 bp upstream of the ATG start codon of HPRT1 gene was electroporated into wild-type (wt) HAP1 cells and led to Cas3-dependent editing of 78% and 34%, respectively (FIGS. 2F-2H). The same HPRT-G1 guide also caused robust DNA targeting in bESCs, HEK293T, and HeLa cells, as evidenced by the smaller-than-wt products in the long-range genomic PCR analysis (FIG. 9B). Moreover, Cascade was successfully reprogrammed to edit another endogenous gene CCR5 in HAP1 cells (FIG. 9C). Altogether, the data established Nla CRISPR-Cas3 as a compact type I-C system repurposed for high-efficiency genome engineering in human cells.

### Example 3

### Nla CRISPR-Cas3 Creates a Spectrum of Large, Unidirectional Genomic Deletions

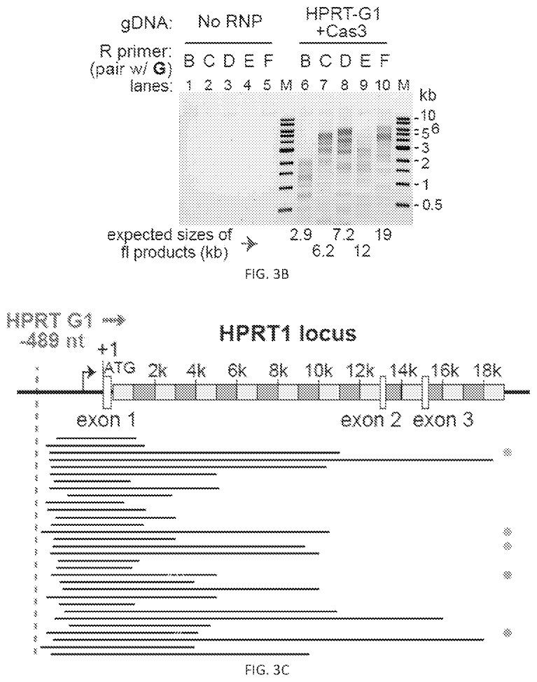

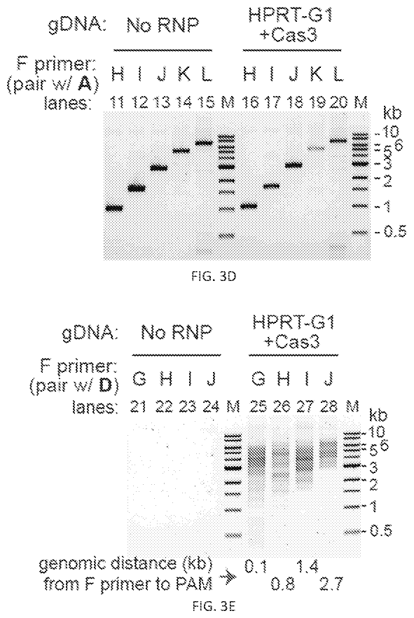

Type I-E CRISPR generates targeted unidirectional large deletions towards the PAM-proximal direction in human. Intriguingly, it was recently shown that the Pae I-C CRISPR forms bidirectional large deletions in various bacteria hosts. Without making presumption about the directionality or size range of the NlaCas3-induced lesions, three different sets of PCRs were performed using genomic DNA extracted from HAP1 cells edited by Cascade-HPRT-G1 and Cas3 from FIG. 2H. First, to specifically amplify regions downstream of CRISPR-programmed site, a fixed forward primer G annealing 100 bp upstream of target site was used, and it was paired with tiling reverse primers B through F about 2.8-19 kb downstream of target (FIG. 3A). Each PCR amplification gave rise to a collection of bands of varying sizes but all smaller than the corresponding full-length product (FIG. 3B, lanes 6-10), indicative of heterogenous large deletions firing downstream in the PAM-proximal direction. The control genomic DNA from untreated cells failed to give any product (FIG. 3B, lanes 1-5), likely due to a GC-rich region in exon 1 that prevented PCR amplification.

To precisely define the boundaries of these NlaCas3-mediated deletions, the PCR products from lanes 6-10 of FIG. 3B were pooled, TOPO-cloned, and 34 independent clones were randomly selected for Sanger sequencing. A total of 31 unique lesions was identified, and the overall pattern was similar to that exhibited by Tfu type I-E Cascade-Cas3. The onset of deletions was not uniformly at the presumed R-loop but instead was clustered in a window ˜15-150 nt downstream, while the deletion endpoints were distributed across the ˜20 kb PAM-proximal genomic region analyzed (FIG. 3C), highlighting the heterogenous nature of the large deletions caused by NlaCas3. Furthermore, vast majority of the resulting chromosomal junctions have the 5′ and 3′ sequences flanking the deletion rejoined seamlessly, presumably by end-joining DNA repair pathways in human cells (FIG. 3C).

Then, the converse PCR experiment was conducted to 210 amplify regions upstream of the CRISPR-targeted site, using a fixed reverse primer A annealing 0.25 kb downstream of the target, in conjunction with serial forward primers H through L about 0.8-6.4 kb upstream of target (FIG. 3A). No obvious large deletions were detected, as the anticipated full-length bands from both edited and untreated cells were observed (FIG. 3D). This observation suggested that there are very few, if any, NlaCas3-induced deletions firing upstream towards the PAM-distal direction. Collectively, the Nla I-C and Tfu I-E systems likely use similar mechanisms for processive DNA degradation by Cas3 and subsequent DNA repair by endogenous machinery of human cells.

In the last set of long-range PCRs, serial forward primers G through J were paired with a common reverse primer D annealing 7.1 kb downstream of target (FIG. 3A), and a spectrum of amplicons containing large deletions were detected (FIG. 3E, lanes 25-28). Of note, the size of the smallest amplicon in each reaction was larger than the genomic distance between CRISPR target site and the annealing position of the forward primer used, implying that very few bidirectional large deletions existed that span both PAM-proximal and PAM-distal regions of the target.

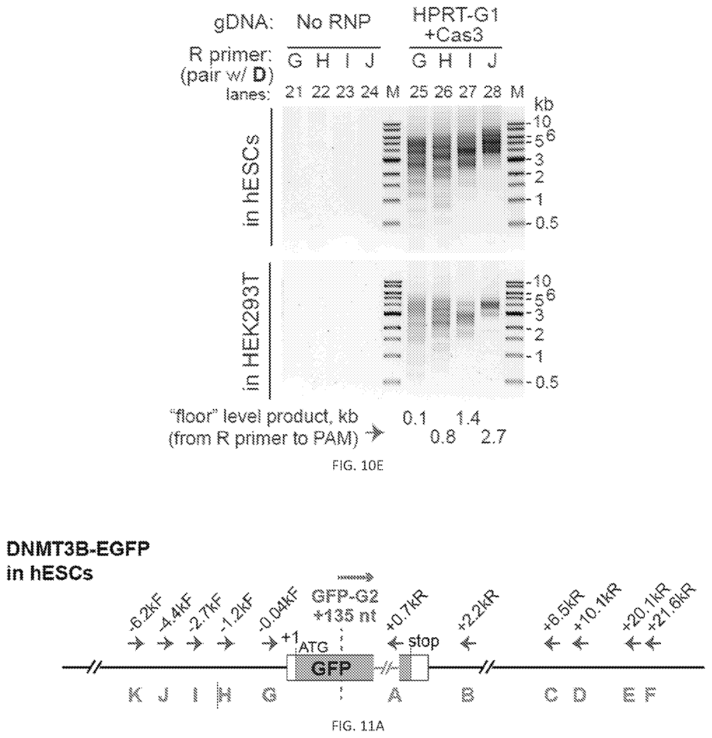

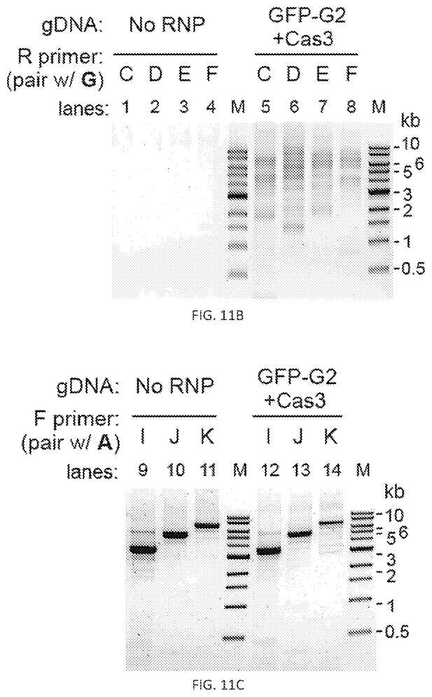

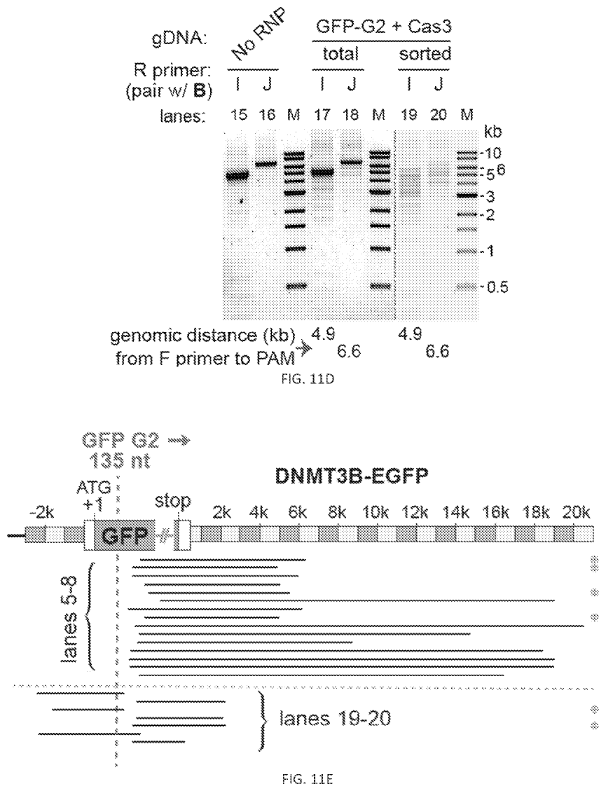

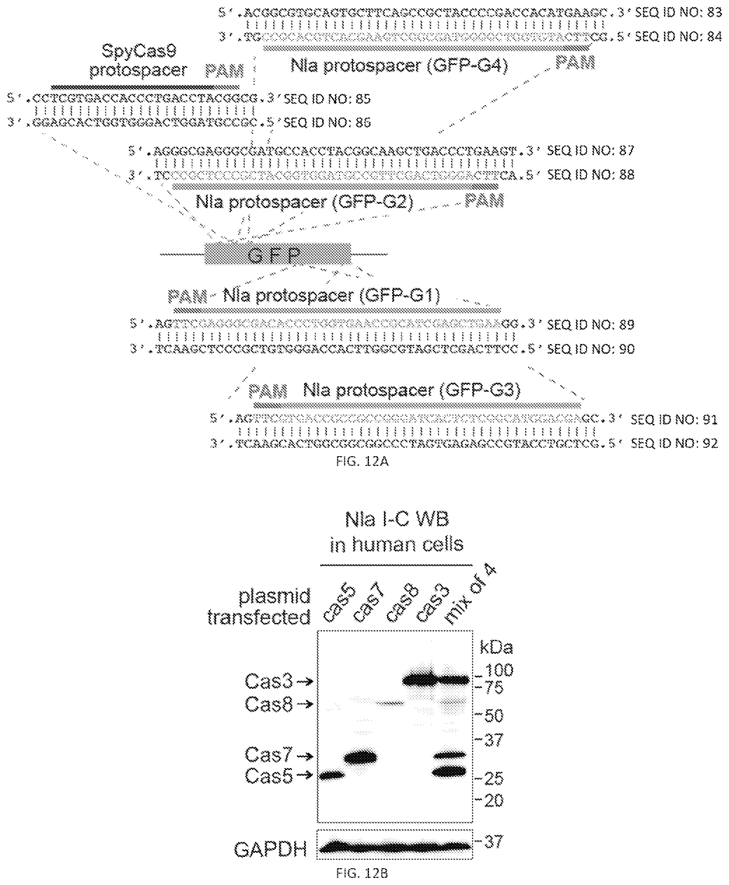

In addition, similar long-range PCR results were observed for RNP editing experiments performed on the same HPRT1 target in hESCs and HEK293T cells (FIG. 10), as well as on the DNMT3b-GFP target in hESCs (FIG. 11), although in the latter experiment a few bidirectional lesions were also detected (FIGS. 11D-E). Taken together, Nla CRISPR-Cas3 created a spectrum of large, unidirectional deletions originating from the target site in human cells.

### Example 4

### Nla CRISPR-Cas3 Encodes a “hidden” cas11 Gene by Alternative Translation Initiation

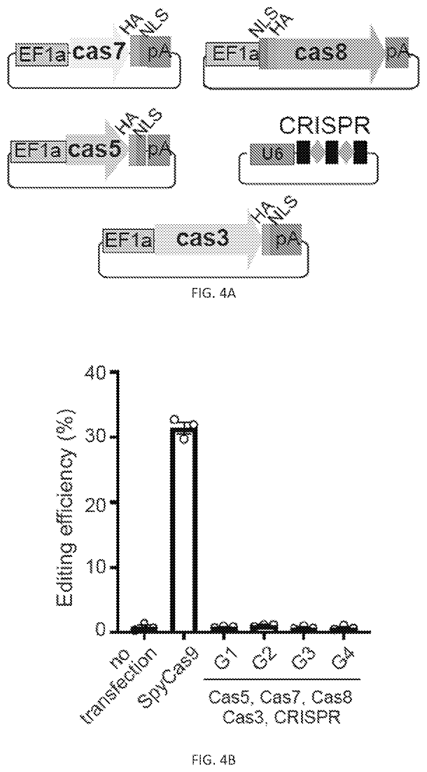

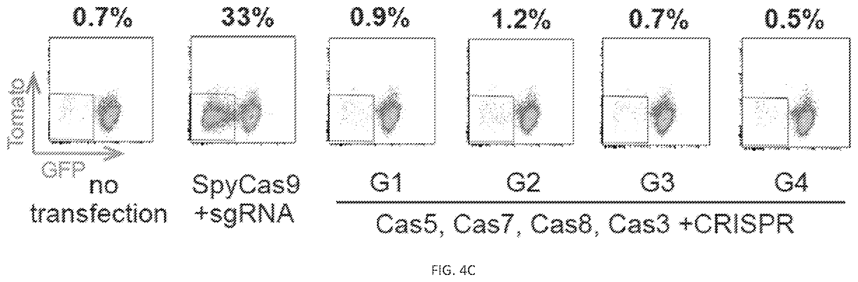

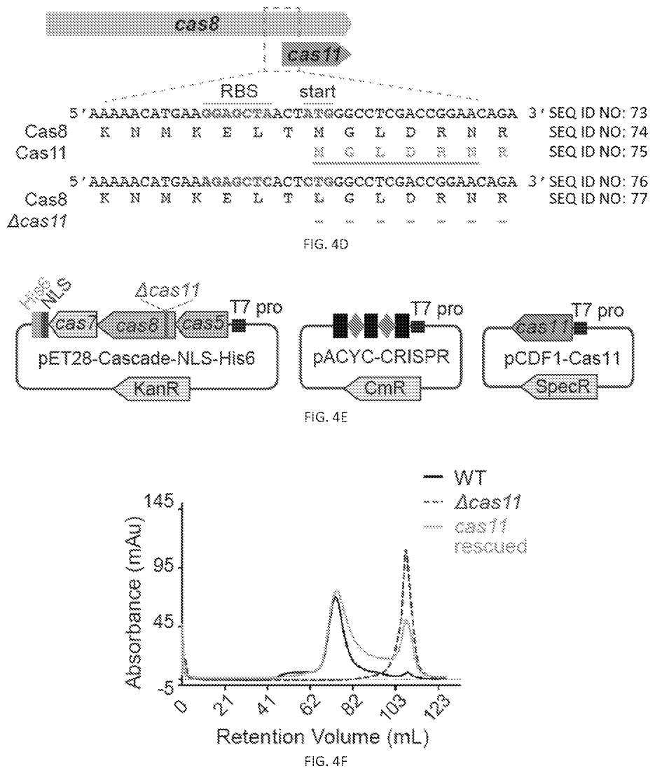

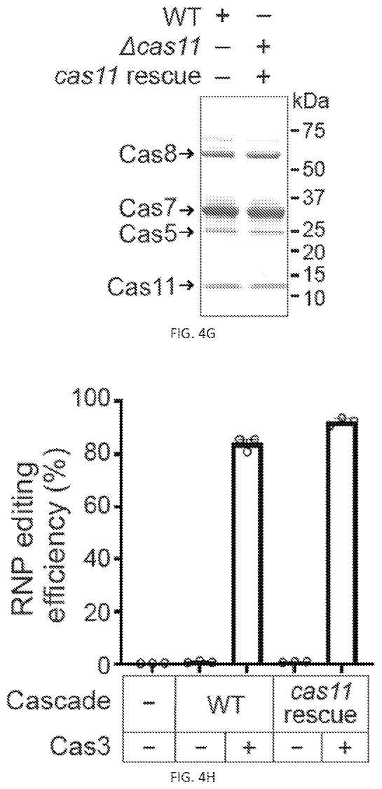

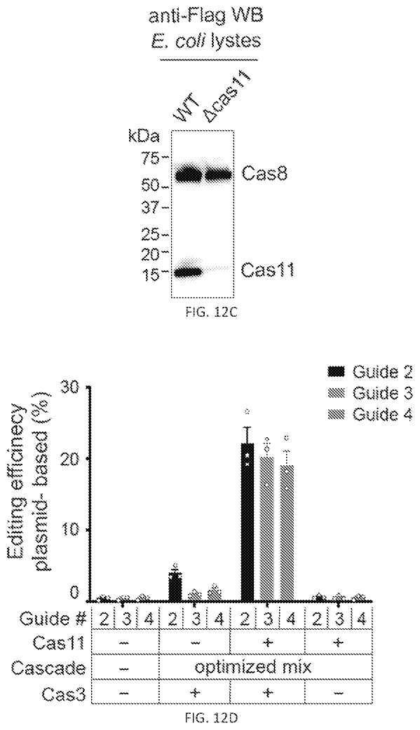

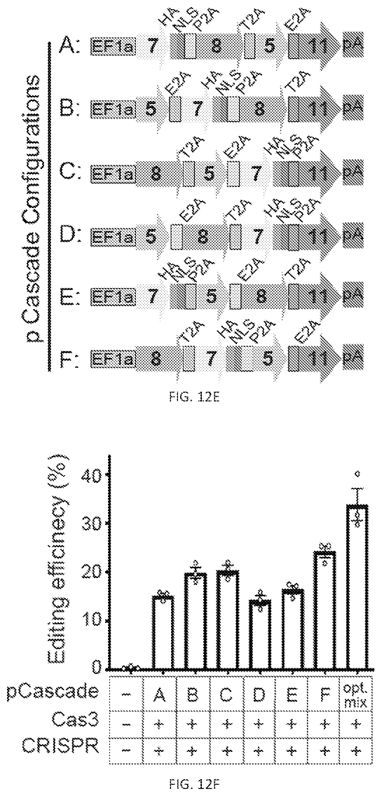

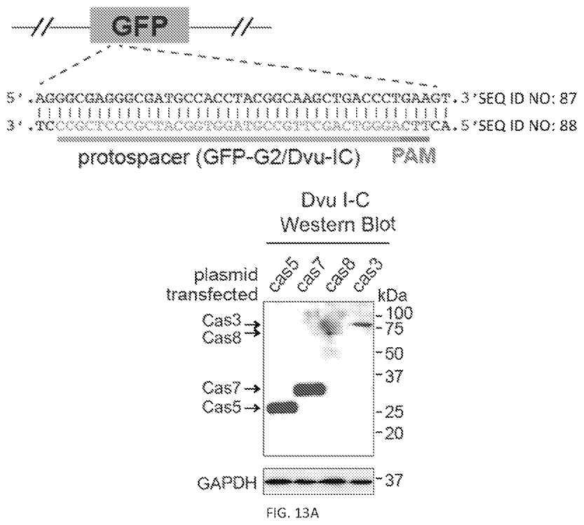

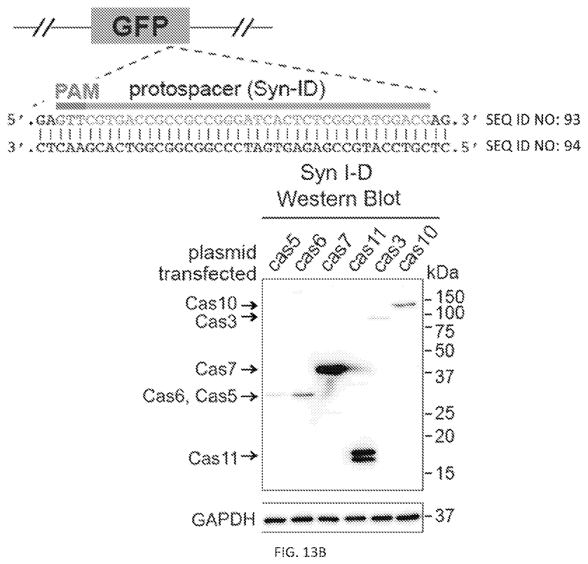

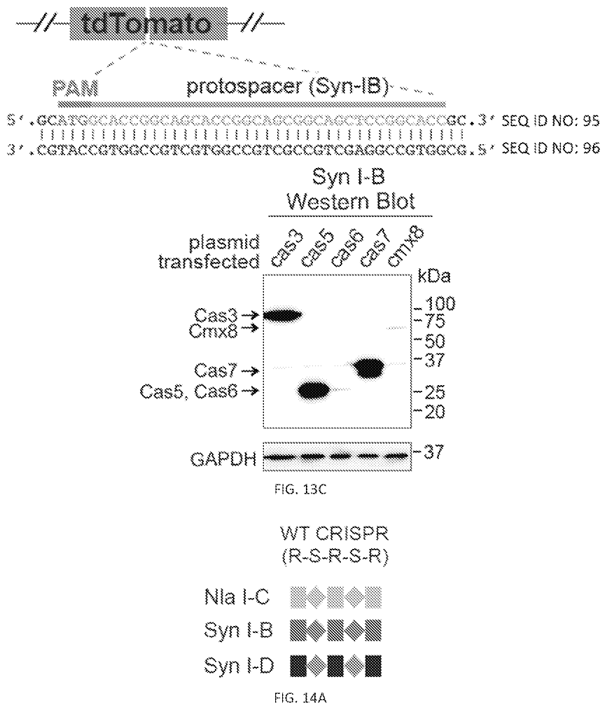

The reprogramming, expression, and purification of Cascade-Cas3 could be laborious or even technically challenging for certain Type I CRISPR systems. A large plasmid-based gene editing platform was designed to facilitate applications involving a large number of individual guide RNAs. All four annotated Nla cas genes were human codon optimized, fused with a NLS, and separately cloned into a mammalian expression vector under control of EF1a promoter and bGH polyA signal (FIG. 4A). A fifth plasmid expressing a mini-CRISPR targeting GFP was co-transfected along with all four cas plasmids into HAP1 reporter cells, and the genome editing activity was evaluated by flow cytometry. A total of four different guides targeting 5′-TTC-flanked sequences in GFP were tested (FIG. 12A), but disappointingly none yielded a positive signal while the SpyCas9 control gave 33% editing (FIGS. 4B-C). The failure in getting the Nla I-C plasmids to edit was not due to the lack of Cas protein expression in human cells, as shown by western blot (FIG. 12B).

To better understand the discrepancy between plasmid- and RNP-editing results, the SDS-PAGE of NlaCasacde purifications was revisited. An unexpected ˜14 kDa protein band consistently showed up in all the purifications (FIG. 2A, marked by star). The N terminus of this extra protein band was sequenced through Edman degradation, and mapped to residue M666 of NlaCas8 protein (FIG. 4D). McBride et al. (Molecular cell (2020) 80, 971-979 e977) uncovered a previously unannotated gene cas11 that is encoded via internal translation from within the large subunit gene cas8 of certain types I-D, I-C, and I-B CRISPR systems. This finding prompted speculation that the 14 kDa peptide in the Cascade prep is the cas11 homolog for Nla I-C system. In silico prediction using the same algorithm identified a high-confidence translation start site at residue M666 of NlaCas8. Further inspection of the sequence immediately upstream of M666 revealed a putative ribosome binding site (RBS) (FIG. 4D). Importantly, internal translation was in-frame with Cas8 and the expected product is 14.7 kDa, consistent with SDS-PAGE result in FIG. 2A. Moreover, when mutations were introduced into the predicted RBS and internal start codon on the Cascade expression vector, Cas11 production in E. coli culture was ablated (FIG. 12C). Collectively, Nla type I-C CRISPR indeed possessed a previously overlooked internal open reading frame (ORF) as Cas11 homolog.

Attempts to purify a cas11 null version of NlaCascade failed, owning to the lack of stable Cascade complex formation during SEC (Δcas11, FIGS. 4D-F). Cas11 complementation from a separate E. coliexpression plasmid restored Cascade formation, resulting in a Cas11 rescue version of NlaCascade that was as effective in directing genome editing as the wt counterpart (both with >80% efficiencies, FIGS. 4D-H). Taken together, NlaCas11 is an integral part of the target recognition module Cascade for genome engineering with Nla CRISPR-Cas3. This is in contrast with the Synechocystis (Syn) Type I-D system where Δcas11 did not prevent Cas5, Cas7 and Cas8 from assembling into a stable complex, although with severely impaired DNA binding capacity. Thus, Cas11 orthologs from different Type I systems can play distinct roles in the assembly or DNA binding of Cascade.

### Example 5

### Cas11 Implements Plasmid- and mRNA-Based Genome Editing with Nla CRISPR-Cas3

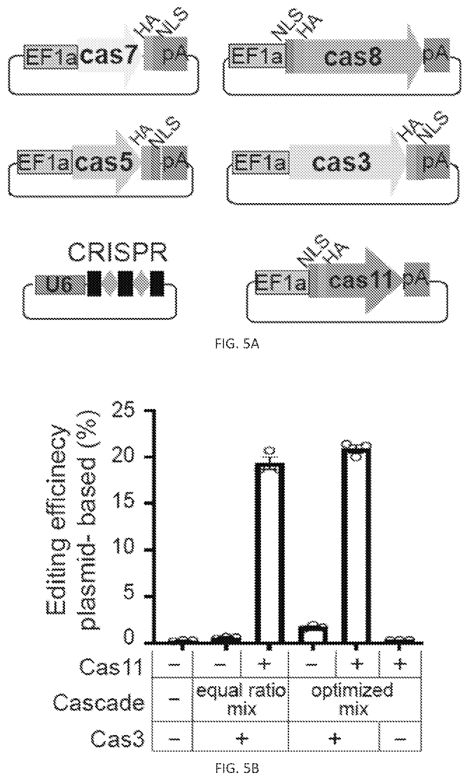

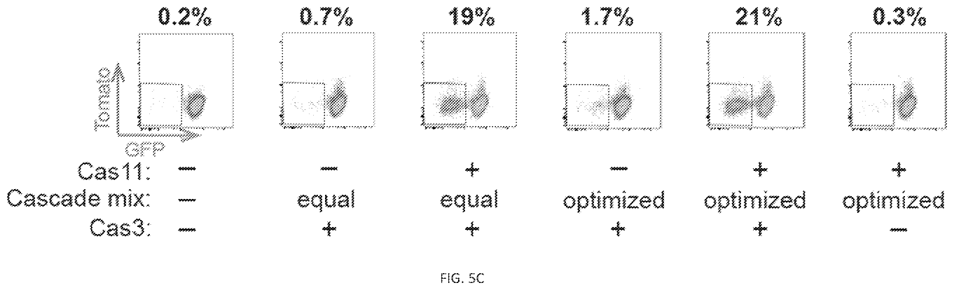

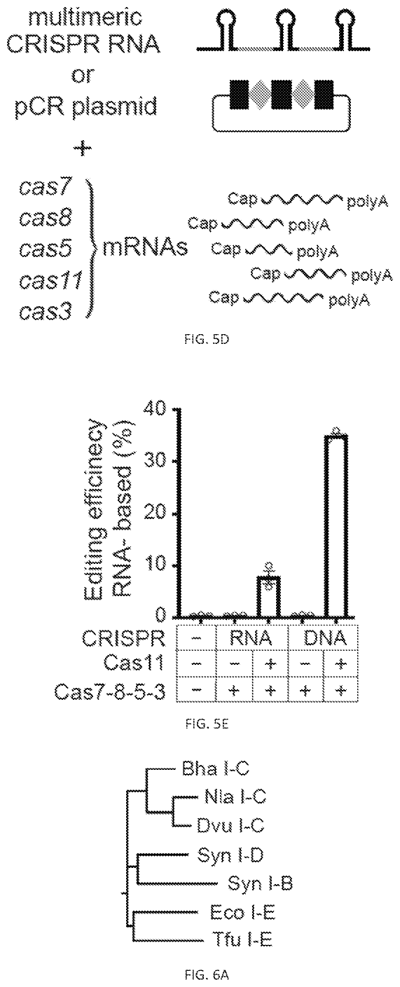

Because prokaryotic and eukaryotic translation machineries operate by distinct mechanisms, the internal prokaryotic promoter embedded within cas8 may not direct Cas11 translation in eukaryotes. Therefore, to establish plasmid-based editing, a separate mammalian expression cassette driving NlaCas11 from a EF1a promoter and Kozak sequence was utilized. A Cas11 vector expressing the Nlacas11 transgene with a N-terminal NLS and a HA tag was transfected into HAP1 reporter cells along with other crispr-cas vectors (FIG. 5A). Remarkably, a mixture of equal amounts of all plasmids exhibited 19% editing while the control mixture lacking pCas11 led to minimal editing (FIGS. 5B-C), demonstrating that Cas11 facilitated robust editing. To optimize editing efficiency, increasing amounts of Cas8 plasmid was used, because Cas8 is the least expressed component in human cells (FIG. 12C). However, the resulting “optimized” plasmid mixture only modestly enhanced the editing rate to 21% (FIGS. 5B-C). The other three GFP-targeting guides gave robust 19-22% editing, but only when Cas11 was included in the optimized plasmid mix (FIG. 12D). Of note, the optimized control lacking pCas11 displayed low but noticeable level of editing around 1-4%, implying that without NlaCas11, the processes of Cascade assembly, Cas3 recruitment and DNA targeting can still occur in human cells, although to a much lesser extent (FIG. 12D).

To streamline applications that would benefit from reduced number of plasmids, all Cascade subunit genes including cas11 were combined into a polycistronic cassette driven from a single EF1a promoter, connected with 2A peptides. The NLS sequences were eliminated from cas8, cas5 and cas11 but not cas7. A panel of such Cascade constructs were created, varying the relative positions of each cas gene in the polycistronic cassette (FIG. 12E). Co-transfection of these Cascade constructs with a pair of Cas3 and CRISPR plasmids resulted in 14-24% editing, with the 8-7-5-11 configuration being the most efficient (FIG. 12F). This set of experiments provided a simplified approach to reconstitute the Nla CRISPR-Cas3 DNA targeting activity in human cells with far fewer plasmids than originally used, while again validating the importance of Cas11.

As RNP- and plasmid-mediated editing was achieved, a third format of delivery via electroporation of messenger RNA (mRNA) was explored. Using in vitro transcribed, 5′ capped and 3′ poly A-tailed mRNAs for cas5, cas7, cas8, cas11, and cas3 genes, along with an in vitro transcribed multimeric pre-CRISPR transcript, ˜8% editing in HAP1 cells was obtained (FIGS. 5F-G). This precursor crRNA which corresponds to the unprocessed transcript derived from CRISPR, was used because its presumed longer half-life in human cells over short mature crRNAs may favor Cascade assembly. Switching from this pre-crRNA to a plasmid-borne CRISPR array substantially boosted the editing efficiency from 8% to 35% (FIGS. 5F-G), likely due to prolonged existence of plasmid versus RNA in the cellular environment. Nonetheless, cas11 mRNA facilitated this RNA-based editing platform, regardless of the form of CRISPR supplied.

### Example 6

### Cas11 Establishes Diverse Miniature CRISPR-Cas3 Orthologs for Gene Editing

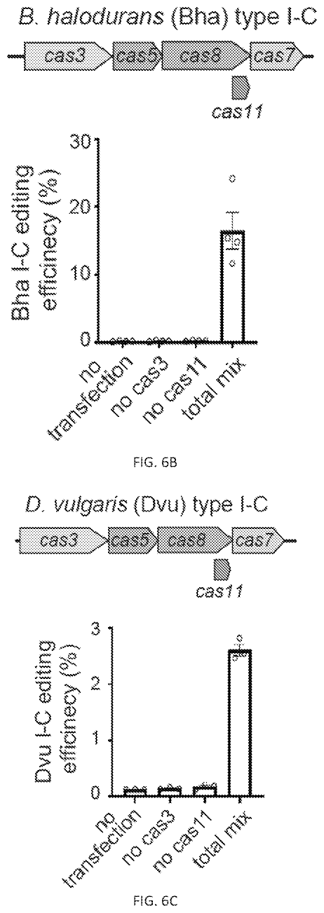

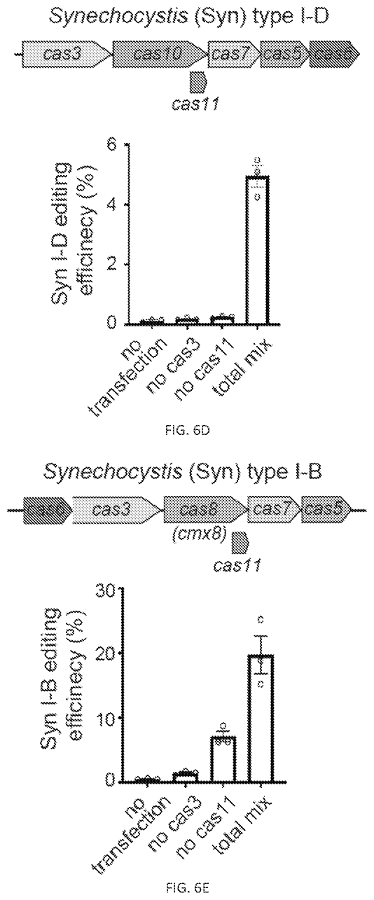

Internal translation of Cas11 in microbes is a conserved phenomenon across many compact CRISPR-Cas3 systems from the I-B, I-C, and I-D subtypes that together encompass nearly a quarter of all native CRISPRs. To test if not having a separately encoded Cas11 limited the utility of diverse miniature CRISPR-Cas3 in eukaryotes, selective orthologs from other species were used (FIG. 6A). In particular, two I-C systems from Bacillus halodurans (Bha) and Desulfovibrio vulgaris (Dvu), both of which have been characterized biochemically and encode their own internal Cas11, were used. For each system, six plasmids expressing a targeting CRISPR and all five cas genes including the predicted non-canonical cas11 were co-transfected into HAP1 cells and displayed 17% and 2.5% editing for Bha and Dvu, respectively (FIGS. 6B-C). Importantly, for both I-C orthologs, Cas11 facilitated effective genome editing (FIGS. 6B-C).

Similar analysis was extended to the I-D and I-B CRISPRs from cyanobacteria Synechocystis (Syn). The Syn I-D system contains five previously annotated cas genes, cas3, cas5, cas6, cas7 and cas10, plus the non-conventional cas11 embedded within cas10. Approximately 5% editing was observed when a Cas11 plasmid was included in the plasmid mixture, but no editing when Cas11 was left out (FIG. 6D). In addition, a putative I-B system from Synechocystis sp. strain PCC 6714 that has not been characterized before was also tested. In-silico prediction identified the potential RBS and alternative start codon for cas11 embedded within the large subunit gene cmx8 (FIG. 6E). Co-expression of the five annotated cas genes cas6, cas3, cmx8, cas7, and cas5 readily induced ˜7% editing, while further addition of the Cas11 plasmid drastically boosted the editing rate to 20% (FIG. 6E). This suggests that although Cas11 is not a prerequisite for Syn I-B CRISPR to achieve Cascade assembly and DNA targeting, its presence can substantially elevate editing efficiency in human cells. Altogether, supplying the hidden cas1l is a broadly applicable approach to exploit divergent miniature CRISPR-Cas3 systems for mammalian genome engineering. Of note, the Syn I-D and I-B editors recognize 5′-GTT and 5′-ATG PAMs respectively, both motifs are different from the 5′-TTC PAM utilized by I-C and the 5′-AAG PAM for I-E editors.

### Example 7

### CRISPR-Cas3 Orthogonality in Human Cells

A myriad of Cas9-based tools has been developed to achieve targeted activities including gene modification, transcription regulation, chromosomal loci imaging, and epigenetic control, and the like. However, any individual Cas9 tool can only mediate one activity at a time in any given cell. Multiple Cas9 proteins can be used concurrently to mediate independent tasks, such as transcription control and gene editing, at different target sites in the same cell. This relies on the orthogonal nature of the Cas9s used, which means that each Cas9 only functions with its own cognate sgRNA. The new set of CRISPR-Cas3 editors established herein opens the possibility for orthogonal Type I applications. However, little is known about the orthogonality barriers separating divergent CRISPR-Cas3 systems, prompting us to examine if their crRNAs are cross-functional in human genome engineering.

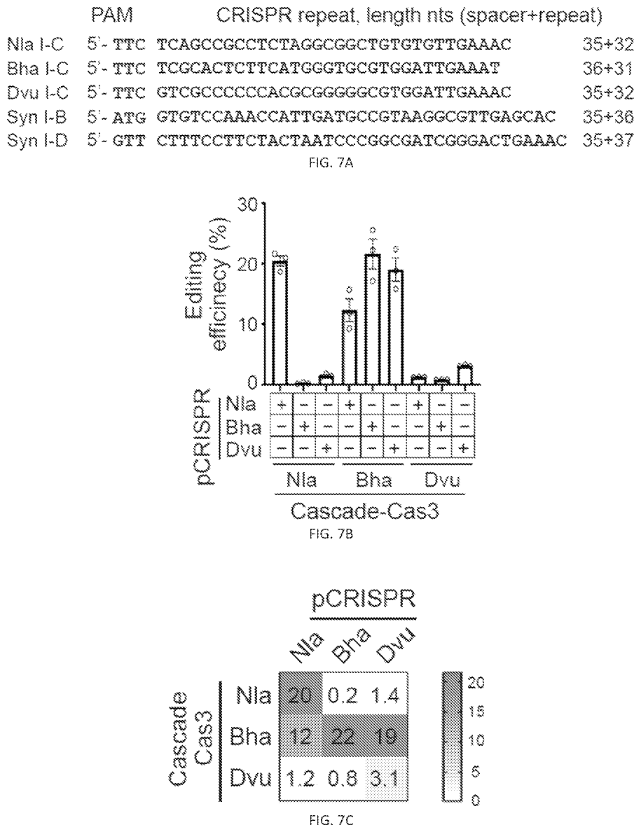

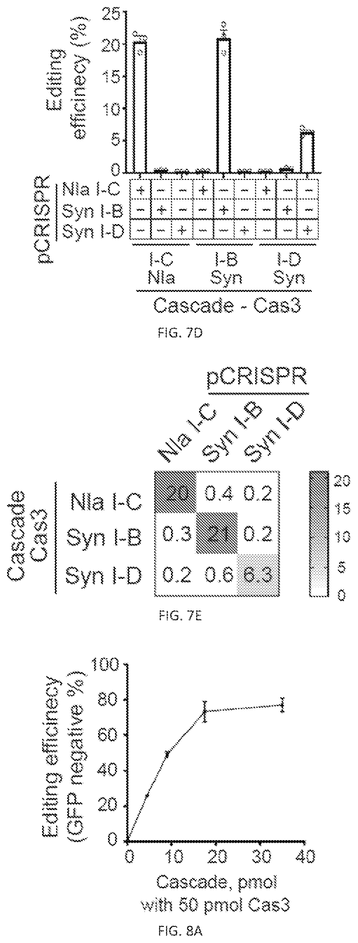

First, a mix-and-match experiment was conducted among the three I-C editors, by assaying each set of the I-C cas genes in conjunction with every I-C CRISPR plasmid. The Nla and Dvu Cas proteins displayed a clear preference for their own respective CRISPR from the same species, while also showing low but noticeable cross-reactivity with each other's CRISPR but no editing at all with Bha CRISPR (FIGS. 7B-C). Unexpectedly, the Bha Cas proteins exhibited robust editing with all three I-C CRISPRs analyzed (12-22% editing, FIGS. 7B-C), revealing high degree of tolerance for crRNAs from other I-C orthologs. Since these three I-C editors lack complete orthogonal barrier in crRNA usage and recognize identical 5′-TTC PAM, they may not be the ideal choice for simultaneous and independent applications.

Next, a similar mix-and-match test was performed among the I-C, I-D, and I-B editors. Each set of the Cas proteins from Nla I-C, Syn I-B, or Syn I-D system functioned exclusively with its own respective CRISPR but not with CRISPRs from the other species, demonstrating true orthogonality (FIGS. 7D-E). For instance, the entire Syn I-B CRISPR-Cas system displayed a robust editing efficiency of 21%; but when its CRISPR was switched to that of the Nla I-C or Syn I-D system, no editing above background was observed, indicating that no CRISPR interchangeability was allowed. The same trend also held true for the Nla I-C and Syn 1-D Cas proteins (FIGS. 7D-E). Such clear orthogonality barrier separating disparate compact CRISPR-Cas3 editors is instrumental for further harnessing them as orthogonal tools for simultaneous and independent Type I applications.

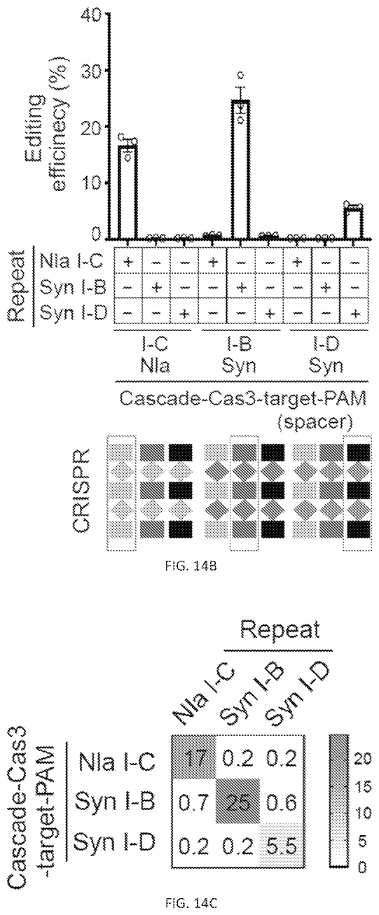

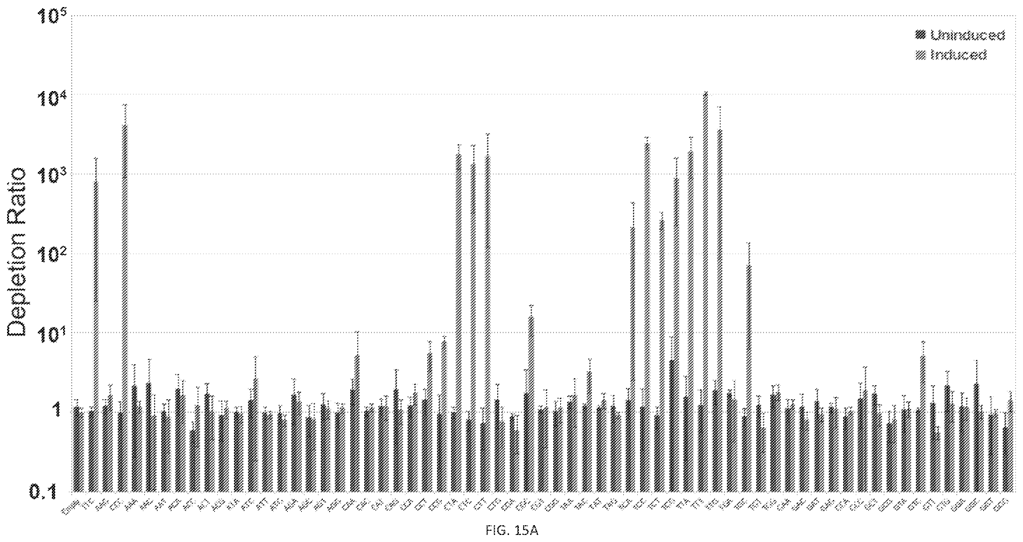

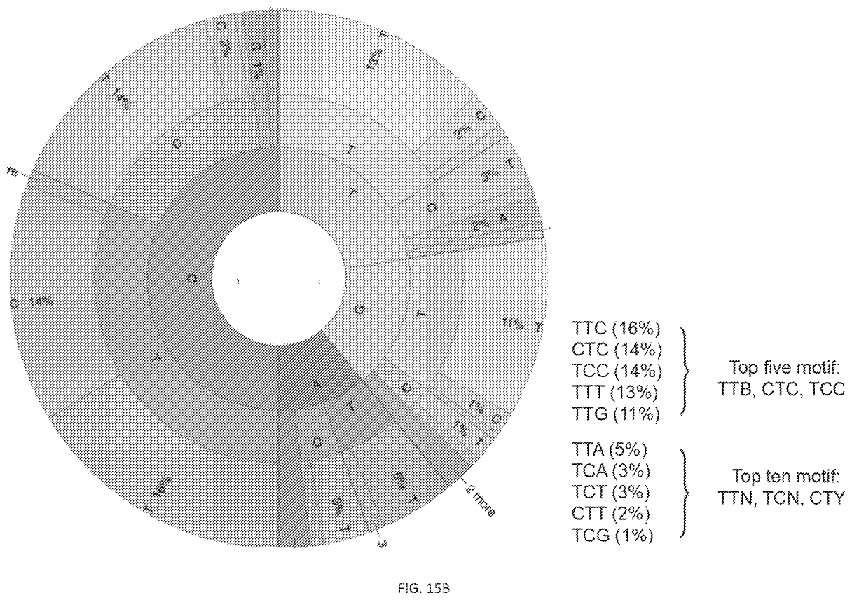

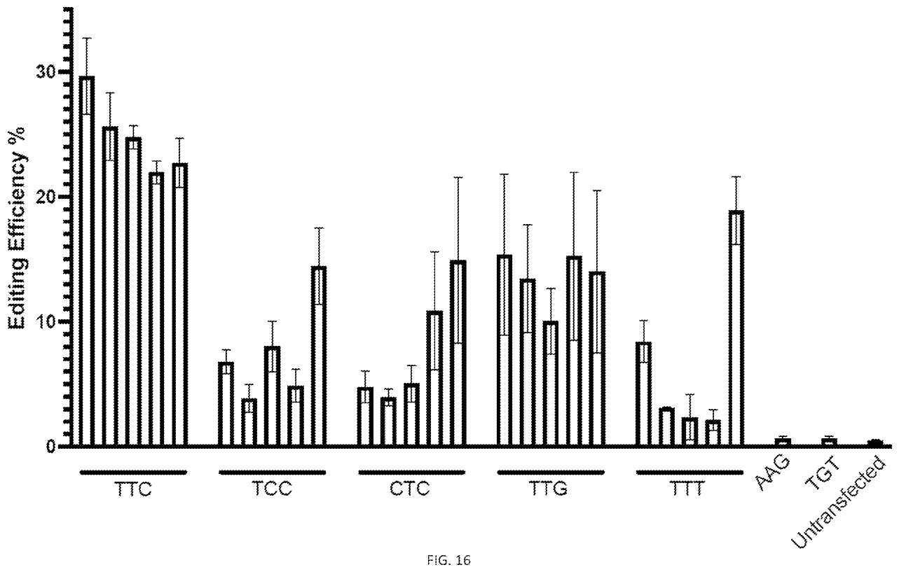

To disentangle the contributions of PAM and crRNA to this orthogonal barrier, chimeric CRISPR constructs in which the original guide/spacer sequence remains unchanged but the repeats are swapped among I-C, I-B and I-D systems were assayed. For example, in the first set of tests the Nla I-C Cas proteins would be directed by its guide to target identical protospacer and 5′-TTC PAM sequence, yet editing only occurred when the respective repeat from the same species was utilized (FIGS. 14A-C). Similarly, Cas proteins from the Syn I-B or I-D system would be guided by their spacers to the respective target site but can only enable editing when paired with the respective repeat (FIGS. 14A-C). These results highlight that the specificity of each Cas protein set for its repeat suffices for orthogonality in genome editing applications.

Sequences:

Nla-cas3 (SEQ ID NO: 99)
GTGAATTTCGACTATATAGCCCACGCTCGCCAAGACTCATCAAAAAATTGGCATTCCCATCC
CCTGCAAAAACATCTACAAAAAGTCGCCCAACTCGCCAAGCGTTTTGCAGGGCGTTATGGGT
CGTTGTTTGCCGAATATGCGGGGCTTTTGCACGATTTGGGGAAATTTCAGGAATCTTTTCAGA
AATATATCCGTAATGCATCCGGCTTTGAAAAAGAAAATGCCCATTTGGAAGATGTCGAATCT
ACCAAGTTGCGCAAAATTCCGCATTCCACTGCCGGTGCCAAATATGCGGTAGAACGTCTAAA
TCCATTTTTCGGGCATTTGCTGGCATATTTGATTGCCGGGCATCATGCTGGGCTGGCAGATTG
GTATGACAAAGGCAGCCTGAAACGCCGTCTGCAACAGGCGGATGACGAGTTGGCAGCGTCT
TTGTCGGGCTTTGTGGAAAGTAGTTTGCCCGAAGATTTTTTCCCGTTATCAGATGATGACTTG
ATGCGGGATTTTTTTGCGTTTTGGGAAGACGGGGCAAAGCTGGAAGAATTGCATATTTGGAT
GCGTTTTCTCTTTTCCTGCTTGGTGGATGCCGATTTTTTGGATACCGAAGCCTTTATGAACGG
CTATGCCGATGCAGATACTGCGCAGGCTGCCGGATTGCGCCCAAAATTTCCCGGTTTGGATG
AGTTACACCGGCGATATGAGCAATATATGGCGCAACTTTCAGAAAAAGCAGATAAAAATTC
ATCTTTAAACCAAGAACGCCACGCCATTTTGCAGCAATGTTTTTCTGCCGCAGAAACGGACC
GTACTTTGTTTTCTTTAACCGTGCCGACCGGTGGCGGTAAAACTTTGGCGAGCTTGGGCTTCG
CTTTGAAGCACGCGCTGAAATTTGGCAAAAAACGTATTATCTATGCTATTCCTTTCACCAGTA
TTATCGAGCAGAATGCCAATGTTTTCCGCAATGCATTAGGCGATGATGTGGTTTTAGAACAC
CACAGCAATTTGGAAGTGAAAGAAGATAAGGAAACAGCGAAAACTCGTCTTGCTACGGAAA
ATTGGGACGCGCCGCTGATTGTTACTACCAATGTGCAACTGTTTGAAAGCCTGTTTGCGGCG
AAAACCAGCCGTTGCCGCAAGATTCACAATATTGCCGACAGCGTGGTGATTTTGGATGAAGC
CCAGCAGCTTCCGCGCGATTTCCAAAAACCGATTACCGACATGATGCGGGTGCTGGCGCGTG
ATTACGGCGTTACCTTTGTGCTGTGCACGGCAACCCAACCGGAGCTTGGCAAAAATATCGAC
GCATTCGGTCGCACTATTTTGGAAGGGCTACCAGATGTGCGCGAAATTGTGGCAGACAAAAT
TGCCTTATCGGAAAAACTGCGCCGCGTCCGCATCAAAATGCCGCCGCCAAACGGCGAAACG
CAAAGCTGGCAGAAAATTGCCGATGAAATAGCCGCGCGCCCGTGTGTTTTGGCAGTGGTCAA
TACGCGAAAACACGCCCAAAAACTCTTTGCCGCCCTGCCTTCTAACGGAATCAAGCTACATT
TATCTGCCAATATGTGCGCCACACACTGCAGCGAAGTGATTGCGTTGGTTCGCCGATATTTG
GCACTGTATCGCGCAGGCAGCCTGCACAAGCCCTTGTGGCTGGTCAGCACGCAGTTGATTGA
AGCAGGCGTGGATTTGGATTTCCCTTGCGTGTATCGGGCGATGGCAGGGCTGGACAGCATTG
CCCAGGCGGCGGGACGGTGCAACCGTGAAGGTAAACTGCCGCAGTTGGGCGAAGTAGTCGT
ATTCCGCGCCGAAGAAGGCGCGCCCAGCGGCAGCCTGAAACAGGGGCAGGACATTACCGAA
GAGATGCTGAAAGCAGGGCTGCTTGATGACCCGCTTTCCCCGTTGGCATTTGCCGAATATTT
CCGCCGATTCAACGGCAAAGGTGATGTGGACAAACACGGTATCACAACGCTTTTGACGGCA
GAAGCATCAAATGAAAATCCGCTGGCAATTAAATTCCGCACAGCTGCCGAACGTTTCCACCT
GATTGATAACCAAGGCGTGGCACTCATTGTGCCGTTTATCCCGTTGGCTCATTGGGAAAAAG
ACGGCAGTCCGCAAATCGTCGAAGCAAACGAGCTGGACGATTTTTTCAGACGACATCTAGAT
GGTGTTGAAGTTTCAGAATGGCAGGATATTTTGGACAAACAACGCTTTCCGCAGCCGCCAGA
CAACTCCTTTGGGCAAACCGATCAACCACTGCTGCCCGAGCCGTTTGAAAGCTGGTTCGGTC
TGTTGGAAAGCGACCCGCTCAAACACAAATGGGTTTACCGCAAGCTGCAACGCTACACGATT
ACTGTGTACGAACACGAACTGAAAAAGTTGCCTGAACATGCCGTTTTTTCAAGAGCGGGATT
GCTCGTGTTAGATAAGGGCTATTACAAAGCCGTGCTTGGCGCGGATTTTGACGATGCGGCTT
GGCTACCTGAAAATTCGGTTTTATGA
human codon optimized Nla-cas3 with NLS and HA tag (SEQ ID NO: 100)
ATGAACTTCGACTATATCGCCCACGCCAGACAGGACAGCAGCAAGAACTGGCACTCTCACCC
TCTGCAGAAACATCTGCAGAAGGTGGCCCAGCTGGCCAAGAGATTTGCCGGCAGATACGGC
AGCCTGTTCGCCGAATATGCCGGCCTGCTGCACGATCTGGGCAAGTTCCAAGAGAGCTTCCA
GAAGTACATCCGGAACGCCAGCGGCTTCGAGAAAGAGAATGCCCACCTGGAAGATGTGGAA
AGCACCAAGCTGCGGAAGATCCCTCACTCTACAGCCGGCGCTAAGTACGCCGTGGAAAGAC
TGAACCCCTTCTTCGGCCATCTGCTGGCCTATCTGATTGCCGGACATCATGCCGGACTGGCCG
ATTGGTACGATAAGGGCAGCCTGAAGCGGAGACTGCAGCAAGCCGATGATGAACTGGCCGC
CTCTCTGTCCGGCTTCGTGGAATCTTCTCTGCCCGAGGACTTCTTCCCTCTGTCCGACGACGA
CCTGATGAGAGACTTCTTCGCCTTCTGGGAGGACGGCGCCAAGCTGGAAGAACTGCACATCT
GGATGCGGTTTCTGTTCAGCTGCCTGGTGGACGCCGACTTCCTGGATACCGAGGCCTTCATG
AACGGCTACGCCGATGCCGATACAGCCCAAGCTGCTGGACTGAGGCCTAAGTTCCCTGGCCT
GGATGAGCTGCATCGGAGATACGAGCAGTACATGGCTCAGCTGTCCGAGAAGGCCGACAAG
AACAGCTCCCTGAATCAAGAGCGGCACGCCATCCTGCAGCAGTGCTTTTCTGCCGCCGAGAC
AGACAGAACCCTGTTCAGCCTGACAGTGCCTACAGGCGGCGGAAAAACTCTGGCCTCTCTGG
GCTTTGCCCTGAAGCACGCCCTGAAGTTCGGCAAGAAGCGGATCATCTACGCCATTCCTTTC
ACCAGCATCATCGAGCAGAACGCCAACGTGTTCAGAAACGCCCTGGGCGACGATGTGGTGC
TGGAACACCACAGCAACCTGGAAGTGAAAGAGGACAAAGAGACAGCCAAGACCAGACTGG
CCACCGAGAATTGGGATGCCCCTCTGATCGTGACCACCAACGTGCAGCTGTTCGAGAGCCTG
TTTGCCGCCAAGACCTCCAGATGCAGAAAGATCCACAATATCGCCGACAGCGTGGTCATCCT
GGACGAAGCTCAGCAGCTGCCCCGGGACTTCCAGAAACCTATCACCGATATGATGCGCGTGC
TGGCCAGAGACTACGGCGTGACCTTTGTGCTGTGTACCGCCACACAGCCTGAGCTGGGCAAG
AACATCGATGCCTTCGGCCGGACCATCCTGGAAGGATTGCCTGACGTGCGGGAAATCGTGGC
CGATAAGATCGCCCTGAGCGAGAAGCTGAGAAGAGTGCGGATCAAGATGCCTCCTCCAAAC
GGCGAGACACAGAGCTGGCAGAAGATCGCCGACGAGATCGCCGCTAGACCATGTGTGCTGG
CCGTGGTCAACACCAGAAAACACGCCCAGAAGCTGTTCGCTGCCCTGCCTAGCAATGGCATC
AAGCTGCACCTGAGCGCCAACATGTGCGCCACACACTGCTCTGAAGTGATCGCCCTCGTGCG
GAGATATCTGGCCCTGTACAGAGCCGGAAGCCTGCACAAACCTCTGTGGCTGGTGTCTACCC
AGCTGATTGAAGCTGGCGTGGACCTGGACTTCCCCTGTGTGTATAGAGCCATGGCCGGCCTG
GATTCTATTGCCCAAGCAGCCGGACGGTGCAACAGAGAGGGAAAACTGCCTCAGCTGGGCG
AAGTGGTGGTGTTCAGAGCTGAAGAAGGCGCCCCTAGCGGCTCTCTGAAGCAAGGCCAGGA
TATCACCGAGGAAATGCTGAAGGCCGGACTGCTGGACGACCCTTTGTCTCCTCTGGCCTTCG
CCGAGTACTTCAGACGGTTCAATGGCAAGGGCGACGTGGACAAGCACGGCATCACAACACT
GCTGACAGCCGAGGCCAGCAACGAGAATCCACTGGCCATCAAGTTCCGGACCGCCGCTGAG
AGATTCCACCTGATCGATAATCAGGGCGTCGCACTGATCGTGCCCTTCATTCCTCTGGCTCAC
TGGGAGAAAGACGGCAGCCCTCAGATCGTGGAAGCCAACGAGCTGGACGATTTCTTCAGGC
GGCACCTGGACGGCGTGGAAGTGTCTGAGTGGCAGGACATCCTGGATAAGCAGCGGTTCCC
TCAGCCTCCTGACAACAGCTTTGGCCAGACCGATCAGCCTCTGCTGCCTGAGCCTTTCGAGA
GTTGGTTCGGCCTGCTCGAGAGCGACCCACTGAAGCACAAATGGGTGTACCGGAAGCTGCA
GCGGTACACCATCACCGTGTATGAGCACGAGCTGAAAAAGCTGCCCGAGCACGCCGTGTTCT
CTAGAGCTGGACTGCTCGTGCTGGACAAGGGCTACTATAAGGCCGTGCTGGGCGCCGATTTT
GACGATGCTGCTTGGCTGCCAGAGAACTCTGTGCTGGGCTCTGTGGGCTACCCCTACGATGT
GCCTGATTACGCCGGCAGCTACCCTGAGTTCCCCAAGAAAAAGCGGAAAGTGTGA
Nla-Cas3 protein sequence (SEQ ID NO: 101)
MNFDYIAHARQDSSKNWHSHPLQKHLQKVAQLAKRFAGRYGSLFAEYAGLLHDLGKFQESFQK
YIRNASGFEKENAHLEDVESTKLRKIPHSTAGAKYAVERLNPFFGHLLAYLIAGHHAGLADWYD
KGSLKRRLQQADDELAASLSGFVESSLPEDFFPLSDDDLMRDFFAFWEDGAKLEELHIWMRFLFS
CLVDADFLDTEAFMNGYADADTAQAAGLRPKFPGLDELHRRYEQYMAQLSEKADKNSSLNQE
RHAILQQCFSAAETDRTLFSLTVPTGGGKTLASLGFALKHALKFGKKRIIYAIPFTSIEQNANVER
NALGDDVVLEHHSNLEVKEDKETAKTRLATENWDAPLIVTTNVQLFESLFAAKTSRCRKIHNIA
DSVVILDEAQQLPRDFQKPITDMMRVLARDYGVTFVLCTATQPELGKNIDAFGRTILEGLPDVRE
IVADKIALSEKLRRVRIKMPPPNGETQSWQKIADEIAARPCVLAVVNTRKHAQKLFAALPSNGIK
LHLSANMCATHCSEVIALVRRYLALYRAGSLHKPLWLVSTQLIEAGVDLDFPCVYRAMAGLDSI
AQAAGRCNREGKLPQLGEVVVFRAEEGAPSGSLKQGQDITEEMLKAGLLDDPLSPLAFAEYFRR
FNGKGDVDKHGITTLLTAEASNENPLAIKFRTAAERFHLIDNQGVALIVPFIPLAHWEKDGSPQIV
EANELDDFFRRHLDGVEVSEWQDILDKQRFPQPPDNSFGQTDQPLLPEPFESWFGLLESDPLKHK
WVYRKLQRYTITVYEHELKKLPEHAVESRAGLLVLDKGYYKAVLGADFDDAAWLPENSVL
Nla-cas5 (SEQ ID NO: 102)
ATGAGGTTCATCCTGGAAATCAGTGGTGATTTGGCATGCTTCACAAGGTCTGAGCTAAAGGT
GGAAAGGGTTAGTTATCCTGTGATAACGCCGTCTGCCGCCAGGAACATCCTAATGGCGATAT
TGTGGAAGCCGGCGATTCGCTGGAAGGTCTTGAAGATAGAAATCCTAAAACCGATTCAGTG
GACGAATATCCGCCGCAACGAAGTGGGAACTAAGATGAGTGAGCGTAGCGGCTCGCTCTAT
ATTGAAGATAACCGCCAGCAGCGCGCATCCATGCTGCTGAAAGACGTTGCCTACCGCATTCA
CGCCGATTTTGACATGACCAGTGAAGCGGGCGAGAGCGACAACTATGTTAAATTTGCCGAA
ATGTTCAAGCGGCGGGCAAAGAAAGGACAATATTTCCACCAACCTTATTTAGGCTGTCGTGA
GTTTCCTTGTGATTTCAGGTTGCTGGAAAAAGCCGAAGATGGATTGCCACTCGAAGACATTA
CCCAAGATTTCGGTTTTATGCTGTATGACATGGATTTCAGCAAATCCGACCCGCGTGATTCCA
ATAACGCCGAGCCGATGTTTTACCAATGCAAAGCGGTAAACGGCGTGATTACCGTGCCGCCT
GCCGACAGCGAGGAGGTGAAACGATGA
human codon optimized Nla-cas5 with NLS and HA tag (SEQ ID NO: 103)
ATGCGGTTCATCCTGGAAATCAGCGGCGACCTGGCCTGCTTCACAAGAAGCGAGCTGAAGGT
CGAGCGGGTGTCATACCCTGTGATCACCCCTAGCGCCGCCAGAAACATCCTGATGGCCATTC
TGTGGAAGCCCGCCATCAGATGGAAGGTGCTGAAGATCGAGATCCTGAAGCCTATCCAGTG
GACCAACATCCGGCGGAACGAAGTGGGCACCAAGATGAGCGAGAGAAGCGGCAGCCTGTA
CATCGAGGACAACAGACAGCAGCGGGCCTCCATGCTGCTGAAGGATGTGGCCTATAGAATC
CACGCCGACTTCGACATGACAAGCGAGGCCGGCGAGAGCGACAACTACGTGAAGTTCGCCG
AGATGTTCAAGCGGAGAGCCAAGAAGGGCCAGTACTTCCACCAGCCTTACCTGGGCTGCAG
AGAGTTCCCCTGCGACTTCAGACTGCTGGAAAAGGCCGAGGATGGCCTGCCTCTGGAAGATA
TCACCCAGGACTTCGGCTTCATGCTGTACGACATGGACTTCAGCAAGAGCGACCCCAGAGAC
AGCAACAACGCCGAGCCTATGTTCTACCAGTGCAAGGCCGTGAACGGCGTGATCACTGTGCC
TCCAGCCGATAGCGAGGAAGTGAAGAGAGGCAGCGTCGGCTACCCCTACGATGTGCCTGAT
TACGCCCCTAAGAAAAAGCGGAAAGTGTGA
Nla-Cas5 protein sequence (SEQ ID NO: 104)
MRFILEISGDLACFTRSELKVERVSYPVITPSAARNILMAILWKPAIRWKVLKIEILKPIQWTNIRRN
EVGTKMSERSGSLYIEDNRQQRASMLLKDVAYRIHADFDMTSEAGESDNYVKFAEMFKRRAKK
GQYFHQPYLGCREFPCDFRLLEKAEDGLPLEDITQDFGFMLYDMDESKSDPRDSNNAEPMFYQC
KAVNGVITVPPADSEEVKR
Nla-cas8 (SEQ ID NO: 105)
ATGATTTTGCACGCGCTCACCCAATACTATCAACGCAAAGCCGAAAGTGATGGCGGTATTGC
CCAGGAAGGGTTTGAAAACAAAGAAATACCGTTCATTATCGTTATAGACAAACAGGGTAAT
TTTATTCAGCTGGAAGATACCCGTGAGCTGAAAGTTAAGAAGAAAGTTGGCCGCACTTTTTT
AGTACCGAAAGGTTTGGGCAGGAGCGGTTCAAAATCCTACGAAGTAAGCAATTTATTGTGG
GATCACTACGGTTATGTACTTGCTTATGCCGGAGAAAAAGGGCAGGAGCAGGCGGACAAAC
AGCATGCCAGCTTTACCGCCAAAGTAAATGAATTGAAACAGGCGCTGCCCGATGATGCAGG
TGTTACTGCGGTTGCTGCCTTTTTGTCTTCTGCGGAAGAAAAAAGCAAAGTCATGCAGGCTG
CAAATTGGGCGGAGTGTGCCAAAGTCAAAGGCTGTAATCTCAGCTTCCGCCTGGTGGATGAA
GCGGTAGATTTGGTTTGCCAGTCAAAGGCGGTGCGGGAATATGTGAGTCAAGCAAATCAAA
CGCAATCCGATAATGTCCAAAAAGGCATTTGCCTGGTAACGGGCAAAGCTGCGCCGATTGCG
CGGCTGCATAACGCCGTGAAAGGCGTGAATGCCAAGCCCGCCCCGTTTGCATCGGTAAATCT
GTCGGCTTTTGAATCATACGGCAAAGAGCAGGGCTTTATCTTTCCCGTGGGCGAGCAAGCCA
TGTTCGAATATACCACCGCCTTGAACACCTTGCTTGCTAGCGAAAACCGATTCCGTATCGGC
GATGTAACGGCCGTATGTTGGGGCGCGAAACGGACTCCGTTGGAGGAAAGTCTTGCTTCGAT
GATTAACGGCGGCGGCAAAGACAAGCCCGATGAGCATATCGATGCCGTTAAAACTCTTTATA
AAAGCCTATACAACGGTCAATACCAAAAACCTGACGGCAAAGAAAAATTCTACCTTTTAGGT
TTATCGCCCAATTCCGCGCGCATTGTCGTCCGCTTTTGGCATGAAACCACCGTTGCCGCCTTA
TCAGAAAGTATTGCGGCGTGGTATGACGATTTGCAAATGGTGCGCGGCGAAAACTCGCCATA
CCCCGAATATATGCCGCTACCGCGCCTGCTGGGTAATTTGGTGTTGGACGGCAAAATGGAAA
ACCTGCCATCTGACCTGATTGCCCAAATAACCGATGCCGCGCTCAACAACCGTGTTTTACCC
GTCAGCCTGTTGCAGGCTGCTTTGCGGCGCAACAAGGCGGAACAGAAAATTACCTATGGCA
GAGCAAGTCTGCTTAAAGCCTATATCAATCGCGCAATCCGTGCGGGTCGTCTGAAAAACATG
AAGGAGCTAACTATGGGCCTAGATAGAAACCGTCAAGACATCGGCTATGTGCTGGGGCGGC
TGTTTGCCGTGCTGGAAAAAATACAAGCCGAGGCCAATCCCGGCTTGAACGCCACCATTGCC
GACCGCTATTTCGGTTCGGCAAGCAGCACACCGATTGCCGTATTCGGCACACTGATGCGCTT
GTTGCCGCACCATTTGAACAAACTGGAATTTGAAGGACGTGCCGTACAACTGCAATGGGAA
ATCCGCCAGATTTTGGAACATTGTCAGAGATTTCCTAACCATTTGAATTTGGAACAGCAAGG
CCTATTTGCCATCGGTTACTACCACGAAACCCAATTCCTGTTTACCAAAGACGCATTGAAAA
ACCTGTTCAACGAAGCGAAAACCGCATAA
human codon optimized Nla-cas8c with NLS and HA tag (SEQ ID NO: 106)
ATGGTGCCCAAGAAAAAGCGGAAGGTGTACCCCTACGACGTGCCCGATTATGCCGGCTCTGT
GGGAATTCTGCACGCCCTGACACAGTACTACCAGCGGAAGGCCGAAAGCGACGGCGGAATT
GCCCAAGAGGGCTTCGAGAACAAAGAGATCCCCTTCATCATCGTGATCGACAAGCAGGGCA
ACTTCATCCAGCTCGAGGACACCCGCGAGCTGAAAGTGAAGAAAAAAGTGGGCCGCACCTT
CCTGGTGCCTAAAGGCCTTGGCAGAAGCGGCAGCAAGAGCTACGAGGTGTCCAACCTGCTG
TGGGACCACTACGGATACGTGCTGGCCTATGCCGGCGAGAAGGGACAAGAACAGGCCGATA
AGCAGCACGCCAGCTTCACCGCCAAAGTGAACGAGCTGAAGCAGGCCCTGCCTGATGATGC
TGGCGTGACAGCTGTGGCCGCCTTTCTGTCTAGCGCCGAAGAGAAGTCCAAAGTGATGCAGG
CCGCCAACTGGGCCGAGTGCGCTAAAGTGAAGGGCTGCAACCTGAGCTTCCGGCTGGTGGA
TGAAGCCGTGGATCTCGTGTGTCAGTCTAAGGCCGTGCGCGAGTATGTGTCCCAGGCCAATC
AGACCCAGAGCGACAACGTGCAGAAAGGCATCTGTCTGGTCACCGGCAAGGCCGCTCCTAT
TGCCAGACTGCACAATGCCGTGAAGGGCGTGAACGCCAAGCCTGCTCCTTTCGCCTCTGTGA
ACCTGAGCGCCTTTGAGAGCTACGGCAAAGAGCAGGGCTTCATCTTCCCTGTGGGAGAGCAG
GCCATGTTCGAGTACACCACCGCTCTGAATACCCTGCTGGCCTCCGAGAACAGATTCCGGAT
CGGAGATGTGACCGCCGTGTGTTGGGGAGCCAAGAGAACACCTCTGGAAGAGTCCCTGGCC
AGCATGATCAATGGCGGCGGAAAGGACAAGCCCGACGAGCACATCGACGCCGTGAAAACCC
TGTACAAGAGCCTGTACAACGGCCAGTACCAGAAGCCTGACGGAAAAGAGAAGTTCTACCT
GCTGGGACTGAGCCCCAACAGCGCCAGAATCGTTGTGCGGTTCTGGCACGAGACAACCGTG
GCTGCCCTGTCTGAGTCTATCGCCGCTTGGTACGACGACCTGCAGATGGTTCGAGGCGAGAA
CAGCCCCTATCCTGAGTACATGCCCCTGCCTAGACTGCTGGGCAACCTGGTGCTGGACGGCA
AGATGGAAAACCTGCCTAGCGACCTGATCGCCCAGATCACAGATGCTGCCCTGAACAACAG
AGTGCTGCCTGTCAGTCTGCTGCAGGCAGCCCTGAGAAGAAACAAGGCCGAGCAGAAGATC
ACCTACGGCAGAGCCAGCCTGCTGAAGGCCTACATCAACCGGGCCATCAGAGCCGGACGGC
TGAAGAACATGAAGGAACTGACCATGGGCCTCGACCGGAACAGACAGGATATCGGCTATGT
GCTGGGCAGACTGTTCGCCGTGCTGGAAAAGATTCAGGCCGAGGCCAATCCTGGCCTGAAC
GCCACAATCGCCGACAGATATTTTGGCAGCGCCAGCAGCACACCTATCGCCGTGTTTGGCAC
CCTGATGAGACTGCTGCCTCACCACCTGAACAAGCTGGAATTCGAGGGCAGAGCCGTGCAG
CTCCAGTGGGAGATCAGACAGATCCTGGAACACTGCCAGCGGTTCCCCAATCACCTGAACCT
GGAACAGCAGGGACTGTTTGCCATCGGCTACTACCACGAGACACAGTTTCTGTTCACCAAGG
ACGCCCTGAAGAACCTGTTCAACGAGGCCAAGACCGCCTGA
Nla-Cas8 protein sequence (SEQ ID NO: 107)
MILHALTQYYQRKAESDGGIAQEGFENKEIPFIIVIDKQGNFIQLEDTRELKVKKKVGRTFLVPKG
LGRSGSKSYEVSNLLWDHYGYVLAYAGEKGQEQADKQHASFTAKVNELKQALPDDAGVTAVA
AFLSSAEEKSKVMQAANWAECAKVKGCNLSFRLVDEAVDLVCQSKAVREYVSQANQTQSDNV
QKGICLVTGKAAPIARLHNAVKGVNAKPAPFASVNLSAFESYGKEQGFIFPVGEQAMFEYTTAL
NTLLASENRFRIGDVTAVCWGAKRTPLEESLASMINGGGKDKPDEHIDAVKTLYKSLYNGQYQK
PDGKEKFYLLGLSPNSARIVVRFWHETTVAALSESIAAWYDDLQMVRGENSPYPEYMPLPRLLG
NLVLDGKMENLPSDLIAQITDAALNNRVLPVSLLQAALRRNKAEQKITYGRASLLKAYINRAIRA
GRLKNMKELTMGLDRNRQDIGYVLGRLFAVLEKIQAEANPGLNATIADRYFGSASSTPIAVFGTL
MRLLPHHLNKLEFEGRAVQLQWEIRQILEHCQRFPNHLNLEQQGLFAIGYYHETQFLFTKDALK
NLFNEAKTA
Nla-cas7 (SEQ ID NO: 108)
ATGACTATTGAAAAACGCTACGACTTTGTCTTTTTATTTGATGTGCAAGACGGCAATCCCAAC
GGCGATCCTGACGCAGGTAACCTGCCGCGTATCGACCCGCAAACCGGCGAAGGTTTGGTAA
CTGATGTTTGCCTGAAACGCAAAGTCCGCAACTTTATCCAAATGACTCAAAATGACGAACAT
CACGACATCTTTATCCGCGAAAAAGGCATTTTGAACAACCTGATTGACGAAGCCCACGAGCA
GGAAAACGTAAAAGGCAAAGAAAAAGGCGAGAAAACCGAAGCTGCCCGCCAATACATGTG
CAGCCGTTATTACGACATCCGCACATTTGGCGCAGTGATGACTACCGGCAAAAATGCAGGAC
AAGTACGCGGTCCCGTGCAACTGACTTTTTCTCGCTCTATTGATCCCATCATGACCTTGGAAC
ACAGCATTACCCGCATGGCGGTTACCAACGAAAAAGATGCCAGTGAAACCGGCGACAACCG
TACAATGGGTCGCAAATTCACCGTCCCCTACGGTCTATACCGCTGCCATGGCTTCATTTCTAC
CCATTTTGCCAAACAAACAGGCTTTTCCGAAAACGATTTAGAGCTGTTTTGGCAGGCACTTG
TCAATATGTTTGACCACGACCATTCCGCCGCACGCGGACAAATGAACGCACGCGGGCTCTAT
GTGTTTGAACACAGCAATAATCTAGGTGATGCGCCTGCTGATAGTCTGTTCAAACGCATTCA
GGTAGTCAAAAAGGACGGTGTAGAAGTAGTAAGGAGTTTTGACGATTATCTTGTCAGCGTAG
ACGATAAGAATCTTGAAGAAACCAAGCTGTTGCGTAAATTAGGC
human codon optimized Nla-cas7 with NLS and HA tag (SEQ ID NO: 109)
ATGACCATCGAGAAGCGCTACGACTTCGTGTTCCTGTTCGACGTGCAAGACGGCAACCCCAA
CGGCGATCCTGATGCCGGAAACCTGCCTAGAATCGACCCTCAGACAGGCGAGGGCCTCGTG
ACAGATGTGTGCCTGAAGCGGAAAGTGCGGAACTTCATCCAGATGACCCAGAACGACGAGC
ACCACGACATCTTCATCAGAGAGAAGGGCATCCTGAACAACCTGATCGACGAGGCCCACGA
GCAAGAGAACGTGAAGGGCAAAGAGAAAGGCGAGAAAACCGAGGCCGCCAGACAGTACAT
GTGCAGCCGGTACTACGACATCAGAACCTTCGGCGCCGTGATGACCACCGGCAAGAATGCT
GGACAAGTGCGGGGACCTGTGCAGCTGACCTTCAGCAGATCCATCGATCCCATCATGACCCT
GGAACACAGCATCACCAGAATGGCCGTGACCAATGAGAAGGACGCCAGCGAAACCGGCGA
CAACAGAACCATGGGCAGAAAGTTCACCGTGCCTTACGGCCTGTACCGGTGCCACGGCTTTA
TCAGCACCCACTTCGCCAAGCAGACCGGCTTCAGCGAGAACGACCTGGAACTGTTTTGGCAG
GCCCTGGTCAACATGTTCGATCACGATCACTCTGCCGCCAGAGGCCAGATGAATGCCAGAGG
ACTGTACGTGTTCGAGCACAGCAACAACCTGGGAGATGCCCCTGCCGACAGCCTGTTCAAGA
GAATCCAGGTGGTCAAGAAAGACGGCGTGGAAGTCGTGCGGAGCTTCGACGATTACCTGGT
GTCCGTGGACGACAAGAACCTGGAAGAGACAAAGCTGCTGCGGAAGCTCGGCGGCTCTGTG
GGCTATCCTTACGACGTGCCAGACTACGCCCCTAAGAAAAAGCGCAAAGTGTGA
Nla-Cas7 protein sequence (SEQ ID NO: 110)
MTIEKRYDFVFLFDVQDGNPNGDPDAGNLPRIDPQTGEGLVTDVCLKRKVRNFIQMTQNDEHHD
IFIREKGILNNLIDEAHEQENVKGKEKGEKTEAARQYMCSRYYDIRTFGAVMTTGKNAGQVRGP
VQLTFSRSIDPIMTLEHSITRMAVTNEKDASETGDNRTMGRKFTVPYGLYRCHGFISTHFAKQTG
FSENDLELFWQALVNMFDHDHSAARGQMNARGLYVFEHSNNLGDAPADSLFKRIQVVKKDGV
EVVRSFDDYLVSVDDKNLEETKLLRKLG
Nla-cas11 DNA sequence (SEQ ID NO: 111)
ATGGGCCTAGATAGAAACCGTCAAGACATCGGCTATGTGCTGGGGCGGCTGTTTGCCGTGCT
GGAAAAAATACAAGCCGAGGCCAATCCCGGCTTGAACGCCACCATTGCCGACCGCTATTTCG
GTTCGGCAAGCAGCACACCGATTGCCGTATTCGGCACACTGATGCGCTTGTTGCCGCACCAT
TTGAACAAACTGGAATTTGAAGGACGTGCCGTACAACTGCAATGGGAAATCCGCCAGATTTT
GGAACATTGTCAGAGATTTCCTAACCATTTGAATTTGGAACAGCAAGGCCTATTTGCCATCG
GTTACTACCACGAAACCCAATTCCTGTTTACCAAAGACGCATTGAAAAACCTGTTCAACGAA
GCGAAAACCGCATAA
Nla-cas11 human codon optimized DNA sequence with NLS and HA tag (SEQ ID NO: 112)
ATGGTGCCCAAGAAAAAGCGGAAGGTGTACCCCTACGACGTGCCCGATTATGCCGGCTCTGT
GGGAGGCCTCGACCGGAACAGACAGGATATCGGCTATGTGCTGGGCAGACTGTTCGCCGTG
CTGGAAAAGATTCAGGCCGAGGCCAATCCTGGCCTGAACGCCACAATCGCCGACAGATATTT
TGGCAGCGCCAGCAGCACACCTATCGCCGTGTTTGGCACCCTGATGAGACTGCTGCCTCACC
ACCTGAACAAGCTGGAATTCGAGGGCAGAGCCGTGCAGCTCCAGTGGGAGATCAGACAGAT
CCTGGAACACTGCCAGCGGTTCCCCAATCACCTGAACCTGGAACAGCAGGGACTGTTTGCCA
TCGGCTACTACCACGAGACACAGTTTCTGTTCACCAAGGACGCCCTGAAGAACCTGTTCAAC
GAGGCCAAGACCGCCTGA
Nla-Cas11 protein sequence (SEQ ID NO: 113)
MGLDRNRQDIGYVLGRLFAVLEKIQAEANPGLNATIADRYFGSASSTPIAVFGTLMRLLPHHLNK
LEFEGRAVQLQWEIRQILEHCQRFPNHLNLEQQGLFAIGYYHETQFLFTKDALKNLFNEAKTA
Nla-CRISPR-repeat (SEQ ID NO: 114)
TCAGCCGCCTCTAGGCGGCTGTGTGTTGAAAC
Nla-IC EGFP targeting guide sequence (SEQ ID NO: 115)
gagggcgacaccctggtgaaccgcatcgagctgaa
Nla-IC tdTomato targeting guide sequence (SEQ ID NO: 116)
aagaccatctacatggccaagaagcccgtgcaact
Nla-IC HPRT1 targeting guide sequence (SEQ ID NO: 117)
ctgactcttggcccagtgcttccccaaacccttaa
Nla-IC CCR5 targeting guide sequence (SEQ ID NO: 118)
ttactgtcccettctgggctcactatgctgccgcc
Syn-IB Cas6 protein sequence (SEQ ID NO: 119)
MNFIDLAFPVKGTVLNADHNYYLYSAIAKEFPILHDLPDLAVNTISGKPDREGKILLVPGSKLWM
RLPIDNITHIYQLAGKKLRIGQYSIELGNPSLHPLEPVESLKARIITIKGHTEPISFLEAVKRQLFALEI
TEGDVGIPANHEGIPKRLTLQIKKPERTYSIVGYSVLLSNLSAEDSLKIQQVGIGGKRRLGCGVFYP
AVKKSTNSGNKKNVEATLG
Human codon optimized Syn-IB cas6 with NLS and HA tag (SEQ ID NO: 120)
ATGGTGCCCAAGAAAAAGCGGAAGGTGTACCCCTACGACGTGCCCGATTATGCCGGCTCTGT
GGGAATGAACTTCATCGACCTGGCTTTCCCCGTGAAGGGCACCGTGCTGAACGCCGACCACA
ACTACTACCTGTACAGCGCCATTGCCAAAGAGTTCCCCATCCTGCACGACCTGCCTGACCTG
GCCGTGAATACCATCAGCGGCAAGCCTGACAGAGAGGGCAAGATCCTGCTGGTGCCTGGCA
GCAAGCTGTGGATGAGACTGCCCATCGACAACATCACCCACATCTACCAGCTGGCCGGCAA
GAAGCTGAGAATCGGCCAGTACTCTATCGAGCTGGGCAACCCCTCTCTGCACCCTCTGGAAC
CTGTGGAAAGCCTGAAGGCCCGGATCATCACCATCAAGGGCCATACCGAGCCTATCAGCTTC
CTGGAAGCCGTGAAGAGACAGCTGTTCGCCCTGGAAATCACCGAGGGCGACGTGGGAATTC
CTGCCAATCACGAGGGCATCCCCAAGAGACTGACCCTGCAGATCAAGAAGCCCGAGCGGAC
CTATAGCATCGTGGGCTACTCTGTGCTGCTGAGCAATCTGAGCGCCGAGGACAGCCTGAAGA
TCCAGCAAGTTGGCATCGGCGGCAAGAGAAGGCTTGGCTGTGGCGTGTTCTACCCCGCCGTG
AAAAAGAGCACCAACTCCGGCAACAAGAAGAACGTCGAGGCCACACTGGGCTGA
Syn-IB cmx8 protein sequence (SEQ ID NO: 121)
MPKTQAEILTLDENLAELPSAQHRAGLAGLILMIRELKKWPWFKIRQKEKDVLLSIENLDQYGAS
IQLNLEGLIALFDLAYLSFTEERKSKSKIKDFKRVDEIEIEENGKNKIQKYYFYDVITPQGGFLAGW
DKSDGQIWLRIWRDMFWSIIKGVPATRNPENNRCGLNLNAGDSFSKDVESVWKSLQNAEKTTG
QSGAFYLGAMAVNAENVSTDDLIKWQFLLHFWAFVAQVYCPYILDKDGKRNFNGYVIVIPDIAN
LEDFCDILPDVLSNRNSKAFGFRPQESVIDVPEQGALELLNLIKQRIAKKAGSGLLSDLIVGVEVIH
AEKQGNSIKLHSVSYLQPNEESVDDYNAIKNSYYCPWFRRQLLLNLVNPKFDLASQSWLKRHPW
YGFGDLLSRIPQRWLKENNSYFSHDARQLFTQKGDFDMTVATTKTREYAEIVYKIAQGFVLSKLS
SKHDLQWSKCKGNPKLEREYNDKKEKVVNEAFLAIRSRTEKQAFIDYFVSTLYPHVRQDEFVDF
AQKLFQDTDEIRSLTLLALSSQYPIKRQGETE
Human codon optimized Syn-IB cmx8 with NLS and HA tag (SEQ ID NO: 122)
ATGGTGCCCAAGAAAAAGCGGAAGGTGTACCCCTACGACGTGCCCGATTATGCCGGCTCTGT
GGGAATGCCTAAGACACAGGCCGAGATCCTGACACTGGACTTCAACCTGGCCGAGCTGCCT
AGCGCTCAGCATAGAGCTGGACTGGCCGGACTGATCCTGATGATCCGCGAGCTGAAGAAGT
GGCCCTGGTTCAAGATCCGGCAGAAAGAAAAGGACGTGCTGCTGAGCATCGAGAACCTGGA
TCAGTACGGCGCCAGCATCCAGCTGAATCTGGAAGGACTGATCGCCCTGTTCGACCTGGCCT
ACCTGAGCTTCACCGAGGAACGGAAGTCCAAGAGCAAGATCAAGGACTTCAAGCGCGTGGA
CGAGATCGAGATCGAAGAGAACGGCAAGAACAAGATCCAGAAGTACTACTTTTACGACGTG
ATAACCCCTCAAGGCGGCTTCCTGGCCGGCTGGGATAAGTCTGATGGACAGATCTGGCTGCG
GATCTGGCGGGACATGTTCTGGTCCATCATCAAGGGCGTGCCCGCCACCAGAAATCCCTTCA
ACAATAGATGCGGCCTGAACCTGAACGCCGGCGACAGCTTTAGCAAGGACGTGGAAAGCGT
GTGGAAGTCCCTGCAGAACGCCGAGAAAACCACAGGACAGAGCGGCGCCTTTTACCTGGGA
GCCATGGCCGTGAATGCCGAGAACGTGTCCACCGACGACCTGATCAAGTGGCAGTTCCTGCT
GCACTTCTGGGCCTTCGTGGCCCAGGTGTACTGCCCCTACATCCTGGACAAGGACGGCAAGC
GGAACTTCAACGGCTACGTGATCGTGATCCCCGATATCGCCAACCTGGAAGATTTCTGCGAC
ATCCTGCCTGACGTGCTGAGCAACAGAAACAGCAAGGCCTTCGGCTTCAGACCCCAAGAGTC
CGTGATCGATGTGCCTGAGCAAGGCGCTCTGGAACTGCTGAACCTCATCAAGCAGCGGATCG
CCAAGAAGGCCGGAAGCGGACTGCTGAGCGATCTGATCGTGGGCGTCGAAGTGATCCACGC
CGAAAAGCAGGGCAACAGCATCAAGCTGCACAGCGTGTCCTACCTGCAGCCTAACGAAGAG
TCTGTGGACGACTACAACGCCATCAAGAACAGCTACTACTGCCCATGGTTCCGGCGGCAGCT
GCTGCTGAATCTCGTGAACCCCAAGTTCGATCTGGCCAGCCAGAGCTGGCTGAAGAGACACC
CTTGGTACGGCTTCGGCGACCTGCTGTCTAGAATCCCTCAGCGGTGGCTGAAAGAGAACAAC
TCCTACTTCAGCCACGACGCCCGGCAGCTGTTTACCCAGAAAGGCGACTTCGACATGACCGT
GGCCACCACCAAGACCAGAGAATACGCCGAGATCGTGTACAAGATCGCCCAGGGCTTCGTG
CTGTCCAAGCTGAGCAGCAAGCACGACCTGCAGTGGTCCAAGTGCAAGGGCAACCCCAAGC
TGGAAAGAGAGTACAACGACAAAAAAGAGAAGGTCGTCAACGAGGCCTTCCTGGCTATCAG
AAGCCGGACAGAGAAGCAGGCCTTCATCGACTACTTCGTGTCCACACTGTACCCTCACGTGC
GGCAGGACGAGTTCGTGGATTTTGCCCAGAAGCTGTTCCAGGACACCGACGAAATCAGATCT
CTGACCCTGCTGGCCCTGTCCTCTCAGTACCCTATCAAGAGACAGGGCGAGACAGAGTGA
Syn-IB Cas7 protein sequence (SEQ ID NO: 123)
MSNLNLFATILTYPAPASNYRGESEENRSVIQKILKDGQKYAIISPESMRNALREMLIELGQPNNR
TRLHSEDQLAVEFKEYPNPDKFADDFLFGYMVAQTNDAKEMKKLNRPAKRDSIFRCNMAVAVN
PYKYDTVFYQSPLNAGDSAWKNSTSSALLHREVTHTAFQYPFALAGKDCAAKPEWVKALLQAI
AELNGVAGGHARAYYEFAPRSVVARLTPKLVAGYQTYGFDAEGNWLELSRLTATDSDNLDLPA
NEFWLGGELVRKMDQEQKAQLEAMGAHLYANPEKLFADLADSFLGV
Human codon optimized Syn-IB cas7 with NLS and HA tag (SEQ ID NO: 124)
ATGAGCAACCTGAACCTGTTCGCCACCATCCTGACATACCCTGCTCCAGCCAGCAACTACAG
AGGCGAGAGCGAAGAGAACAGAAGCGTGATCCAGAAGATCCTGAAGGACGGCCAGAAGTA
CGCCATCATCAGCCCCGAGAGCATGCGGAATGCCCTGAGAGAGATGCTGATCGAGCTGGGC
CAGCCTAACAACCGGACAAGACTGCACAGCGAGGATCAGCTGGCCGTGGAATTCAAAGAGT
ACCCCAATCCTGACAAGTTCGCCGACGACTTCCTGTTCGGCTACATGGTGGCCCAGACCAAC
GACGCCAAAGAGATGAAGAAGCTGAACAGACCCGCCAAGCGGGACAGCATCTTCAGATGCA
ATATGGCCGTGGCCGTGAATCCCTATAAGTACGACACCGTGTTCTATCAGAGCCCTCTGAAC
GCCGGCGATAGCGCCTGGAAGAATAGCACATCTAGCGCCCTGCTGCACCGGGAAGTGACCC
ATACAGCCTTTCAGTACCCCTTCGCTCTGGCCGGCAAAGACTGTGCCGCTAAGCCTGAATGG
GTCAAAGCTCTGCTGCAGGCCATTGCCGAGCTGAATGGTGTTGCTGGCGGACACGCCAGAGC
CTACTATGAGTTTGCCCCTAGAAGCGTGGTGGCCCGGCTGACACCTAAACTGGTGGCCGGCT
ATCAGACCTACGGCTTCGATGCCGAAGGCAACTGGCTGGAACTGAGCAGACTGACCGCCAC
CGACAGCGACAATCTGGACCTGCCTGCCAACGAGTTTTGGCTCGGCGGCGAACTCGTGCGGA
AGATGGATCAAGAGCAGAAGGCCCAGCTGGAAGCCATGGGAGCCCACCTGTATGCCAATCC
AGAGAAGCTGTTCGCCGATCTGGCCGACTCTTTCCTGGGCGTGGGCAGCGTCGGCTACCCCT
ACGATGTGCCTGATTACGCCCCTAAGAAAAAGCGGAAAGTGTGA
Syn-IB cas5 protein sequence (SEQ ID NO: 125)
MAQLALALDTVTRYLRLKAPFAAFRPFQSGSFRSTTPVPSFSAVYGLLLNLAGIEQRQEVEGKVT
LIKPKAELPKLAIAIGQVKPSSTSLINQQLHNYPVGNSGKEFASRTFGSKYWIAPVRREVLVNLDLI
IGLQSPVEFWQKLDQGLKGETVINRYGLPFAGDNNFLFDEIYPIEKPDLASWYCPLEPDTRPNQG
ACRLTLWIDRENNTQTTIKVFSPSDFRLEPPAKAWQQLPG
Human codon optimized Syn-IB cas5 with NLS and HA tag (SEQ ID NO: 126)
ATGGCTCAACTGGCCCTGGCTCTGGATACCGTGACCAGATACCTGAGACTGAAGGCCCCTTT
CGCCGCCTTCAGACCTTTTCAGAGCGGCAGCTTCCGGTCCACCACACCTGTGCCATCTTTCAG
CGCCGTGTATGGCCTGCTGCTGAATCTGGCCGGAATCGAGCAGCGGCAAGAGGTGGAAGGC
AAAGTGACCCTGATCAAGCCCAAGGCCGAGCTGCCTAAACTGGCCATTGCCATCGGCCAAGT
GAAGCCCAGCAGCACCAGCCTGATCAACCAGCAGCTGCACAACTACCCCGTGGGCAACAGC
GGCAAAGAGTTCGCCAGCAGAACCTTCGGCAGCAAGTACTGGATCGCCCCTGTGCGGAGAG
AGGTGCTGGTCAACCTGGATCTGATCATCGGCCTGCAGAGCCCCGTGGAATTCTGGCAGAAA
CTGGACCAGGGCCTGAAGGGCGAGACAGTGATCAACAGATACGGCCTGCCTTTTGCCGGCG
ACAACAACTTCCTGTTCGACGAGATCTACCCCATCGAGAAGCCCGATCTGGCCAGCTGGTAC
TGCCCTCTGGAACCCGACACCAGACCTAATCAGGGCGCCTGTAGACTGACCCTGTGGATCGA
CAGAGAGAACAACACCCAGACCACCATCAAGGTGTTCAGCCCCAGCGACTTCCGGCTGGAA
CCTCCTGCTAAAGCTTGGCAGCAGCTCCCTGGAGGCAGCGTCGGCTACCCCTACGATGTGCC
TGATTACGCCCCTAAGAAAAAGCGGAAAGTGTGA
Syn-IB cas11 protein sequence (SEQ ID NO: 127)
MTVATTKTREYAEIVYKIAQGFVLSKLSSKHDLQWSKCKGNPKLEREYNDKKEKVVNEAFLAIR
SRTEKQAFIDYFVSTLYPHVRQDEFVDFAQKLFQDTDEIRSLTLLALSSQYPIKRQGETE
Human codon optimized Syn-IB cas11 (SEQ ID NO: 128)
ATGACCGTGGCCACCACCAAGACCAGAGAATACGCCGAGATCGTGTACAAGATCGCCCAGG
GCTTCGTGCTGTCCAAGCTGAGCAGCAAGCACGACCTGCAGTGGTCCAAGTGCAAGGGCAA
CCCCAAGCTGGAAAGAGAGTACAACGACAAAAAAGAGAAGGTCGTCAACGAGGCCTTCCTG
GCTATCAGAAGCCGGACAGAGAAGCAGGCCTTCATCGACTACTTCGTGTCCACACTGTACCC
TCACGTGCGGCAGGACGAGTTCGTGGATTTTGCCCAGAAGCTGTTCCAGGACACCGACGAAA
TCAGATCTCTGACCCTGCTGGCCCTGTCCTCTCAGTACCCTATCAAGAGACAGGGCGAGACA
GAGTGA
Syn-IB Cas3 protein sequence (SEQ ID NO: 129)
MLKQLLAKSLPTDPQKKPLSLEQHLLDTETAALVIFKGRMLDNWCRFFKVKDPDEFLLHLRVAA
LFHDLGKANHEFIEAVTAKGFVPQTLRHEWISALVLHLPEVRQWLGKSNLNLEVVTAAVLSHHL
KASPDGDYKWDEPQKSGDKVETKLYFNHEEVDRILNKIANLLDVDSKLPELPKKWIKGDIFLENI
YKDANQIGRKFTRQAKKDDSLKGLLLAVKAGLIASDSVASGIYRTQDSEAIANWVNQTLHTNSIT
PEBIEEKILHPRYRQVEKSINEPFQLKRFQEKAETLSSRLLLMSGCGSGKTIFAYKWMQGVLNKH
QAGRAIFLYPTRGTATEGFKDYVSWCPEADASLLTGTATYELQAIAKNPTEANEGKDYQADERL
YALGYWGKRFFSATVDQFLSFLTHNYKSICLLPVLADSVVVIDEIHSFSPEMFDSLVCFLKTFDVP
VLCMTATLPQTRIEDLTIQLDKDKDGLGLEVFPTSDRSELAELEKAEGMERYLIAHTNEEAALDL
AVKAYQDSKRVLWVVNTVDRCREKARKLECLLKTEVLTYHSRFKLADRQNRHRETVEAFALH
QAQGEKKAAIAVTTQVCEMSLDLDADVLITELAPISSLVQRFGRSNRGDKNDKTEPSKIYVYKPP
KDKPYKQKDDLDPAEKFINDVLGRASQKLLAEKLKEHSPPGRYSDGSAPFVTQGYWASSDEPFR
KIDDFAVNAVLTEDLGEITQYLNSNPPKPIDGFIVPVPKKYKFQGFSHRPPQLPKYLEIADSKFYSS
KRGFGDDA
Human codon optimized Syn-IB cas3 with NLS and HA tag (SEQ ID NO: 130)
ATGCTGAAACAGCTGCTGGCCAAGAGCCTGCCTACCGATCCTCAGAAGAAGCCCCTGAGCCT
GGAACAGCATCTGCTGGACACAGAGACAGCCGCTCTGGTCATCTTCAAGGGCAGAATGCTG
GACAACTGGTGCCGGTTCTTCAAAGTGAAGGACCCCGACGAGTTCCTGCTGCACCTGAGAGT
GGCCGCTCTGTTTCACGATCTGGGCAAAGCCAACCACGAGTTCATCGAGGCCGTGACCGCCA
AGGGATTCGTGCCTCAGACACTGAGACACGAGTGGATCTCTGCCCTGGTGCTGCATCTGCCT
GAAGTTCGACAGTGGCTGGGCAAGAGCAACCTGAACCTGGAAGTGGTTACAGCCGCCGTGC
TGAGCCACCACCTGAAAGCTTCTCCCGACGGCGACTACAAGTGGGACGAGCCTCAGAAAAG
CGGCGACAAGGTGGAAACAAAGCTGTACTTCAACCACGAAGAGGTGGACCGGATCCTGAAC
AAGATCGCCAACCTGCTGGACGTGGACAGCAAGCTGCCTGAGCTGCCCAAGAAGTGGATCA
AGGGCGACATCTTCCTGGAAAACATCTACAAGGACGCCAACCAGATCGGCCGGAAGTTCAC
CAGACAGGCCAAGAAGGACGACAGCCTGAAGGGACTGCTGCTGGCTGTGAAGGCCGGACTG
ATCGCCTCTGATTCTGTGGCCAGCGGCATCTACAGAACCCAGGACTCTGAGGCCATTGCCAA
CTGGGTCAACCAGACACTGCACACCAACAGCATCACCCCTGAGGAAATCGAGGAAAAGATT
CTGCACCCTCGGTACAGACAGGTGGAAAAGAGCATCAACGAGCCCTTCCAGCTGAAGCGGT
TCCAAGAGAAGGCCGAGACTCTGAGCAGTCGGCTGCTGCTGATGTCTGGCTGTGGCTCTGGC
AAGACCATCTTCGCCTATAAGTGGATGCAGGGCGTGCTGAACAAGCACCAGGCCGGCAGAG
CCATCTTTCTGTACCCTACAAGAGGCACCGCCACCGAGGGCTTCAAGGACTATGTGTCCTGG
TGTCCTGAGGCCGATGCCTCTCTGCTGACTGGCACAGCCACATACGAGCTGCAGGCTATCGC
CAAGAATCCCACCGAGGCCAACGAGGGCAAAGACTACCAGGCCGACGAGAGACTGTACGCC
CTCGGCTATTGGGGCAAGAGATTCTTCAGCGCTACCGTGGACCAGTTCCTGAGCTTTCTGAC
CCACAACTACAAGAGCATCTGTCTGCTGCCCGTGCTGGCCGATAGCGTGGTGGTTATCGATG
AGATCCACAGCTTCAGCCCCGAGATGTTCGACAGCCTCGTGTGCTTCCTGAAAACCTTCGAC
GTGCCAGTGCTGTGCATGACCGCTACACTGCCCCAGACCAGAATCGAGGACCTGACCATCCA
GCTCGACAAGGACAAGGATGGCCTGGGCCTCGAGGTGTTCCCTACCTCTGATAGAAGCGAG
CTGGCCGAGCTGGAAAAGGCCGAAGGCATGGAAAGATACCTGATCGCCCACACCAACGAGG
AAGCTGCCCTGGATCTGGCCGTGAAAGCCTACCAGGATAGCAAGAGGGTGCTGTGGGTCGT
GAACACCGTGGACAGATGCAGAGAGAAAGCCCGGAAGCTGGAATGCCTGCTGAAAACCGA
GGTGCTGACCTACCACAGCCGGTTCAAACTGGCCGACCGGCAGAACAGACACCGGGAAACC
GTGGAAGCCTTCGCTCTGCATCAGGCCCAGGGCGAGAAAAAAGCCGCCATTGCCGTGACCA
CACAAGTGTGCGAGATGTCCCTGGACCTGGACGCCGATGTGCTGATCACAGAGCTGGCCCCT
ATCAGCAGCCTGGTGCAGAGATTCGGCAGAAGCAACCGGGGCGACAAGAACGACAAGACC
GAGCCTAGCAAGATCTACGTGTACAAGCCTCCTAAGGACAAGCCCTACAAGCAGAAGGATG
ATCTGGACCCCGCCGAGAAGTTCATCAACGACGTTCTGGGCAGAGCCTCTCAGAAGCTCCTG
GCCGAGAAGCTGAAAGAGCACAGCCCTCCAGGCAGATACTCCGATGGATCTGCCCCTTTCGT
GACCCAAGGCTACTGGGCCTCTAGCGACGAGCCTTTCAGAAAGATCGACGACTTCGCCGTGA
ACGCAGTGCTGACAGAGGATCTGGGCGAGATCACCCAGTACCTGAACAGCAACCCTCCTAA
GCCTATCGACGGCTTCATCGTGCCCGTGCCTAAGAAGTACAAGTTCCAGGGCTTCAGCCACC
GGCCACCTCAGCTGCCTAAGTACCTGGAAATCGCCGACAGCAAGTTCTACAGCAGCAAGAG
AGGCTTTGGCGACGACGCCGGCAGCGTCGGCTACCCCTACGATGTGCCTGATTACGCCCCTA
AGAAAAAGCGGAAAGTGTGA
Syn-IB CRISPR repeat sequence (SEQ ID NO: 131)
GTGTCCAAACCATTGATGCCGTAAGGCGTTGAGCAC
Syn-IB tdTomato targeting guide sequence (SEQ ID NO: 132)
GCACCGGCAGCACCGGCAGCGGCAGCTCCGGCACC
Syn-ID Cas10 protein sequence (SEQ ID NO: 133)
MTTLLQTLLIRTLSEQKDYILLEYFQTILPALEEHFGNTSGLGGSFISHQKHFGTQGYDTEKAKKM
AQGFAKKGDQTLAAHILNALLTTWNVMQELEFPLNDIERRLLCLGITLHDYDKHCHAQDMAAP
EPDNIQEINICLELGKRLNFDEFWADWRDYIAEISYLAQNTHGKQHTNLISSNWSNAGYPFTIKE
RKLDHPLRHLLTFGDVAVHLSSPHDLVSSTMGDRLRDLLNRLGIEKRFVYHHLRDTTGILSNAIH
NVILRTVQKLDWKPLLFFÅQGVIYFAPQDTEIPERNEIKQIVWQGISQELGKKMSAGDVGFKRDG
KGLKVSPQTSELLAAADIVRILPQVISVKVNNAKSPATPKRLEKLELGDAEREKLYEVADLRCDR
LAELLGLVQKEIFLLPEPFIEWVLKDLELTSVIMPEETQVQSGGVNYGWYRVAAHYVANHATWD
LEEFQEFLQGFGDRLATWAEEEGYFAEHQSPTRQIFEDYLDRYLEIQGWESDHQAFIQELENYVN
AKTKKSKQPICSLSSGEFPSEDQMDSVVLFKPQQYSNKNPLGGGQIKRGISKIWSLEMLLRQAFW
SVPSGKFEDQQPIFIYLYPAYVYAPQVVEAIRELVYGIASVNLWDVRKHWVNNKMDLTSLKSLP
WLNEEVEAGTNAQLKYTKEDLPFLATVYTTTREKTDTDAWVKPAFLALLLPYLLGVKAIATRS
MVPLYRSDQDFRESIHLDGVAGFWSLLGIPTDLRVEDITPALNKLLAIYTLHLAARSSPPKARWQ
DLPKTVQEVMTDVLNVFALAEQGLRREKRDRPYESEVTEYWQFAELFSQGNIVMTEKLKLTKR
LVEEYRRFYQVELSKKPSTHAILLPLSKALEQILSVPDDWDEEELILQGSGQLQAALDRQEVYTRP
IIKDKSVAYETRQLQELEAIQIFMTTCVRDLFGEMCKGDRAILQEQRNRIKSGAEFAYRLLALEAQ
QNQN
Human codon optimized Syn-ID cas10 with NLS and HA tag (SEQ ID NO: 134)
ATGGTGCCCAAGAAAAAGCGGAAGGTGTACCCCTACGACGTGCCCGATTATGCCGGCTCTGT
GGGAATGACCACACTGCTGCAGACACTGCTGATCCGGACCCTGTCCGAGCAGAAGGACTAC
ATCCTGCTGGAGTATTTCCAGACAATCCTGCCAGCCCTGGAGGAGCACTTTGGAAACACCAG
CGGACTGGGAGGATCCTTCATCTCTCACCAGAAGCACTTTGGCACACAGGGCTACGACACCG
AGAAGGCCAAGAAGATGGCCCAGGGCTTCGCCAAGAAGGGCGATCAGACACTGGCCGCCCA
CATCCTGAACGCCCTGCTGACCACATGGAATGTGATGCAGGAGCTGGAGTTTCCTCTGAACG
ATATCGAGCGGAGACTGCTGTGCCTGGGCATCACCCTGCACGACTATGATAAGCACTGTCAC
GCACAGGACATGGCAGCACCAGAGCCAGATAACATCCAGGAGATCATCAATATCTGCCTGG
AGCTGGGCAAGAGGCTGAATTTCGACGAGTTTTGGGCCGACTGGCGCGATTACATCGCCGAG
ATCAGCTATCTGGCCCAGAACACACACGGCAAGCAGCACACCAATCTGATCAGCTCCAACTG
GTCCAATGCCGGCTACCCCTTCACAATCAAGGAGCGGAAGCTGGATCACCCTCTGAGACACC
TGCTGACCTTTGGCGACGTGGCCGTGCACCTGTCTAGCCCTCACGATCTGGTGTCCTCTACCA
TGGGCGACAGGCTGCGCGATCTGCTGAACAGGCTGGGCATCGAGAAGCGGTTCGTGTACCA
CCACCTGCGGGACACCACAGGCATCCTGAGCAACGCCATCCACAATGTGATCCTGAGAACA
GTGCAGAAGCTGGACTGGAAGCCACTGCTGTTCTTTGCCCAGGGCGTGATCTATTTCGCCCC
ACAGGATACCGAGATCCCCGAGAGAAATGAGATCAAGCAGATCGTGTGGCAGGGCATCTCT
CAGGAGCTGGGCAAGAAGATGAGCGCCGGCGACGTGGGCTTTAAGAGGGATGGCAAGGGC
CTGAAGGTGTCCCCACAGACATCTGAGCTGCTGGCAGCAGCAGACATCGTGCGCATCCTGCC
CCAGGTCATCTCCGTGAAGGTGAACAATGCCAAGTCTCCCGCCACCCCTAAGCGGCTGGAGA
AGCTGGAGCTGGGCGATGCCGAGAGAGAGAAGCTGTACGAGGTGGCCGACCTGCGGTGCGA
TAGACTGGCCGAGCTGCTGGGCCTGGTGCAGAAGGAGATCTTCCTGCTGCCTGAGCCCTTCA
TCGAGTGGGTGCTGAAGGACCTGGAGCTGACATCTGTGATCATGCCAGAGGAGACCCAGGT
GCAGAGCGGAGGCGTGAACTACGGCTGGTATCGGGTGGCCGCCCACTACGTGGCCAATCAC
GCCACCTGGGACCTGGAGGAGTTCCAGGAGTTTCTGCAGGGCTTCGGCGATAGACTGGCCAC
ATGGGCCGAGGAGGAGGGCTATTTCGCCGAGCACCAGTCTCCTACCCGGCAGATCTTTGAGG
ACTACCTGGATAGATATCTGGAGATCCAGGGCTGGGAGAGCGACCACCAGGCCTTTATCCAG
GAGCTGGAGAACTACGTGAATGCCAAGACCAAGAAGTCCAAGCAGCCAATCTGTTCTCTGA
GCTCCGGCGAGTTCCCCAGCGAGGACCAGATGGATTCCGTGGTGCTGTTTAAGCCCCAGCAG
TATTCCAACAAGAATCCTCTGGGAGGAGGACAGATCAAGAGGGGAATCAGCAAGATCTGGT
CCCTGGAGATGCTGCTGAGACAGGCCTTCTGGAGCGTGCCCTCCGGCAAGTTCGAGGACCAG
CAGCCTATCTTTATCTACCTGTATCCTGCCTACGTGTATGCCCCACAGGTGGTGGAGGCCATC
AGGGAGCTGGTGTACGGCATCGCCAGCGTGAACCTGTGGGACGTGCGCAAGCACTGGGTGA
ACAATAAGATGGATCTGACATCTCTGAAGAGCCTGCCTTGGCTGAACGAGGAGGTGGAGGC
CGGCACAAATGCCCAGCTGAAGTACACCAAGGAGGACCTGCCATTCCTGGCCACCGTGTATA
CCACAACCAGGGAGAAGACAGACACCGATGCCTGGGTGAAGCCAGCCTTTCTGGCCCTGCT
GCTGCCATACCTGCTGGGAGTGAAGGCAATCGCAACCCGCTCTATGGTGCCCCTGTATCGGA
GCGACCAGGATTTCAGAGAGTCCATCCACCTGGATGGAGTGGCAGGATTTTGGTCCCTGCTG
GGAATCCCTACAGACCTGAGGGTGGAGGATATCACCCCAGCCCTGAATAAGCTGCTGGCCAT
CTATACCCTGCACCTGGCAGCAAGGTCTAGCCCACCTAAGGCAAGGTGGCAGGACCTGCCCA
AGACAGTGCAGGAAGTGATGACCGATGTGCTGAACGTGTTCGCACTGGCAGAGCAGGGACT
GAGGAGGGAGAAGAGGGACAGACCTTACGAGTCCGAGGTGACAGAGTATTGGCAGTTCGCC
GAGCTGTTTTCTCAGGGCAATATCGTGATGACAGAGAAGCTGAAGCTGACCAAGCGCCTGGT
GGAGGAGTACCGGCGGTTCTACCAGGTGGAGCTGTCTAAGAAGCCTAGCACCCACGCCATC
CTGCTGCCACTGAGCAAGGCCCTGGAGCAGATCCTGTCCGTGCCAGACGATTGGGATGAGG
AGGAGCTGATCCTGCAGGGATCCGGACAGCTGCAGGCCGCCCTGGACAGGCAGGAGGTGTA
CACACGCCCCATCATCAAGGATAAGTCTGTGGCCTATGAGACCAGGCAGCTGCAGGAGCTG
GAGGCAATCCAGATCTTCATGACAACCTGCGTGAGGGACCTGTTTGGCGAGATGTGCAAGG
GCGATCGCGCCATCCTGCAGGAGCAGAGGAACCGCATCAAGAGCGGCGCCGAGTTCGCCTA
CAGACTGCTGGCCCTGGAGGCCCAGCAGAACCAGAATTAA
Syn-ID Cas7 protein sequence (SEQ ID NO: 135)
MLDSLKSQFQPSFPRLASGHYVHFLMLRHSQSFPVFQTDGVLNTTRTQAGLLEKTDQLSRLVMF
KRKQTTPERLAGRELLRNLGLTSADKSAKNLCEYNGEGSCKQCPDCILYGFAIGDSGSERSKVYS
DSAFSLGAYEQSHRSFTFNAPFEGGTMSEAGVMRSAINELDHILPEVTFPTVESLRDATYEGFIYV
LGNLLRTKRYGAQESRTGTMKNHLVGIVFADGEIFSNLHLTQALYDQMGGELNKPISELCETAA
TVAQDLLNKEPVRKSELIFGAHLDTLLQEVNDIYQNDAELTKLLGSLYQQTQDYATEFGALSGG
KKKAKS
Human codon optimized Syn-ID cas7 with NLS and HA tag (SEQ ID NO: 136)
ATGCTGGACAGCCTGAAGTCCCAGTTCCAGCCAAGCTTTCCTAGGCTGGCATCCGGACACTA
CGTGCACTTTCTGATGCTGCGGCACAGCCAGTCCTTCCCCGTGTTTCAGACCGACGGCGTGCT
GAACACCACAAGGACCCAGGCAGGACTGCTGGAGAAGACAGATCAGCTGTCCAGACTGGTC
ATGTTCAAGAGGAAGCAGACCACACCTGAGAGGCTGGCAGGAAGGGAGCTGCTGAGGAATC
TGGGCCTGACATCCGCCGACAAGTCTGCCAAGAACCTGTGCGAGTACAATGGCGAGGGCAG
CTGCAAGCAGTGTCCAGACTGCATCCTGTATGGCTTCGCCATCGGCGATTCTGGCAGCGAGA
GAAGCAAGGTGTACTCCGATTCTGCCTTTTCCCTGGGCGCCTATGAGCAGTCTCACAGGAGC
TTCACCTTTAACGCCCCCTTCGAGGGCGGCACAATGTCTGAGGCCGGCGTGATGCGCAGCGC
CATCAATGAGCTGGACCACATCCTGCCAGAGGTGACCTTCCCCACAGTGGAGTCCCTGCGGG
ATGCCACCTACGAGGGCTTTATCTATGTGCTGGGCAACCTGCTGCGGACAAAGAGATACGGC
GCCCAGGAGTCTAGAACCGGCACAATGAAGAACCACCTGGTGGGCATCGTGTTCGCCGACG
GCGAGATCTTTAGCAATCTGCACCTGACCCAGGCCCTGTATGATCAGATGGGCGGCGAGCTG
AACAAGCCTATCTCCGAGCTGTGCGAGACCGCAGCAACAGTGGCACAGGACCTGCTGAATA
AGGAGCCAGTGAGGAAGTCTGAGCTGATCTTTGGCGCCCACCTGGATACCCTGCTGCAGGAG
GTGAACGACATCTATCAGAATGATGCCGAGCTGACAAAGCTGCTGGGCAGCCTGTACCAGC
AGACCCAGGATTATGCAACAGAGTTCGGCGCCCTGTCCGGAGGCAAGAAGAAGGCCAAGTC
TGGCTCTGTGGGCTATCCTTACGACGTGCCAGACTACGCCCCTAAGAAAAAGCGCAAAGTGT
GA
Syn-ID Cas5 protein sequence (SEQ ID NO: 137)
MTKIYRCKLTLHDNVFFASREMGILYETEKYFHNWALSYAFFKGTIIPHPYGLVGQNAQTPAYLD
RDREQNLLHLNDSGIYVFPAQPIHWSYQINTFKAAQSAYYGRSVQFGGKGATKNYPINYGRAKE
LAVGSEFLTYIVSQKELDLPVWIRLGKWSSKIRVEVEAIAPDQIKTASGVYVCNHPLNPLDCPAN
QQILLYNRVVMPPSSLFSQSQLQGDYWQIDRNTFLPQGFHYGATTAIAQDSPQLSLLDTN
Human codon optimized Syn-ID cas5 with NLS and HA tag (SEQ ID NO: 138)
ATGGTGCCCAAGAAAAAGCGGAAGGTGTACCCCTACGACGTGCCCGATTATGCCGGCTCTGT
GGGAATGACCAAGATCTATCGGTGCAAGCTGACACTGCACGACAACGTGTTCTTTGCCTCTA
GAGAGATGGGCATCCTGTACGAGACAGAGAAGTATTTTCACAATTGGGCCCTGTCCTACGCC
TTCTTTAAGGGCACCATCATCCCACACCCCTATGGACTGGTGGGACAGAACGCACAGACACC
AGCATACCTGGACAGGGATAGAGAGCAGAACCTGCTGCACCTGAATGATAGCGGCATCTAC
GTGTTCCCTGCCCAGCCAATCCACTGGTCCTATCAGATCAATACCTTTAAGGCCGCCCAGTCT
GCCTACTATGGCAGGAGCGTGCAGTTCGGAGGCAAGGGAGCCACAAAGAACTACCCTATCA
ATTATGGAAGGGCAAAGGAGCTGGCAGTGGGATCCGAGTTTCTGACCTACATCGTGTCTCAG
AAGGAGCTGGACCTGCCCGTGTGGATCAGGCTGGGCAAGTGGAGCTCCAAGATCAGGGTGG
AGGTGGAGGCAATCGCACCAGACCAGATCAAGACCGCCAGCGGCGTGTACGTGTGCAACCA
CCCCCTGAATCCTCTGGATTGTCCCGCCAACCAGCAGATCCTGCTGTACAATCGGGTGGTCA
TGCCCCCTTCTAGCCTGTTCTCCCAGTCTCAGCTGCAGGGCGACTATTGGCAGATCGATCGGA
ACACATTCCTGCCACAGGGCTTTCACTACGGAGCAACCACAGCAATCGCACAGGACAGCCC
ACAGCTGTCCCTGCTGGATACCAATTGA
Syn-ID Cas6 protein sequence (SEQ ID NO: 139)
MEDDRYSLYSVVIELGAAKKGFPTGILGRALHSQVLEWLKIGEPSLAEELHQSQISPFSISPLIGKR
RSKLTEEGDRFFFRISLLNGSLLQPLLKGLEQQDKQIVMLDKFAFRLCHIHILPGSHSLARASSYAL
TTQAPTSSKITLKFHSATSFKIDRNTIQPFPLGDSVENSLLRRWNHFAPEELYFPSVSWQIPVAAFE
LKTYSVQLKKSEIGSEGWVTYLFPDQEQAKIASVLSQFAFFAGVGRKTSMGMGQVSVNNHG
Human codon optimized Syn-ID cas6 with NLS and HA tag (SEQ ID NO: 140)
ATGGTGCCCAAGAAAAAGCGGAAGGTGTACCCCTACGACGTGCCCGATTATGCCGGCTCTGT
GGGAATGTTTGACGATCGGTACTCCCTGTATTCTGTGGTCATCGAGCTGGGAGCAGCCAAGA
AGGGATTCCCAACCGGCATCCTGGGCAGAGCCCTGCACTCTCAGGTGCTGGAGTGGCTGAAG
ATCGGAGAGCCATCCCTGGCAGAGGAGCTGCACCAGAGCCAGATCTCCCCATTTTCCATCTC
TCCCCTGATCGGCAAGCGGAGAAGCAAGCTGACAGAGGAGGGCGACCGGTTCTTTTTCAGA
ATCTCCCTGCTGAACGGCTCTCTGCTGCAGCCCCTGCTGAAGGGCCTGGAGCAGCAGGACAA
GCAGATCGTGATGCTGGATAAGTTTGCCTTCAGGCTGTGCCACATCCACATCCTGCCAGGAA
GCCACTCCCTGGCAAGGGCCAGCTCCTACGCCCTGACCACACAGGCCCCTACCTCTAGCAAG
ATCACACTGAAGTTTCACTCTGCCACCAGCTTCAAGATCGACAGGAATACAATCCAGCCCTT
TCCTCTGGGCGATAGCGTGTTCAACTCCCTGCTGAGGCGCTGGAATCACTTTGCCCCTGAGG
AGCTGTATTTCCCATCTGTGAGCTGGCAGATCCCTGTGGCCGCCTTCGAGCTGAAGACCTACT
CCGTGCAGCTGAAGAAGTCTGAGATCGGCAGCGAGGGCTGGGTGACATATCTGTTTCCAGAT
CAGGAGCAGGCCAAGATCGCCTCCGTGCTGTCTCAGTTCGCCTTTTTCGCAGGAGTGGGAAG
GAAGACCAGCATGGGCATGGGCCAGGTGTCCGTGAACAATCACGGCTGA
Syn-ID Cas11 protein sequence (SEQ ID NO: 141)
MTEKLKLTKRLVEEYRRFYQVELSKKPSTHAILLPLSKALEQILSVPDDWDEEELILQGSGQLQA
ALDRQEVYTRPIIKDKSVAYETRQLQELEAIQIFMTTCVRDLFGEMCKGDRAILQEQRNRIKSGAE
FAYRLLALEAQQNQN
Human codon optimized Syn-ID cas11 (SEQ ID NO: 142)
ATGACAGAGAAGCTGAAGCTGACCAAGCGCCTGGTGGAGGAGTACCGGCGGTTCTACCAGG
TGGAGCTGTCTAAGAAGCCTAGCACCCACGCCATCCTGCTGCCACTGAGCAAGGCCCTGGAG
CAGATCCTGTCCGTGCCAGACGATTGGGATGAGGAGGAGCTGATCCTGCAGGGATCCGGAC
AGCTGCAGGCCGCCCTGGACAGGCAGGAGGTGTACACACGCCCCATCATCAAGGATAAGTC
TGTGGCCTATGAGACCAGGCAGCTGCAGGAGCTGGAGGCAATCCAGATCTTCATGACAACCT
GCGTGAGGGACCTGTTTGGCGAGATGTGCAAGGGCGATCGCGCCATCCTGCAGGAGCAGAG
GAACCGCATCAAGAGCGGCGCCGAGTTCGCCTACAGACTGCTGGCCCTGGAGGCCCAGCAG
AACCAGAATTAA
Syn-ID Cas3 protein sequence (SEQ ID NO: 143)
MSLPKVGAMKVQVKPLYAKLNEGLGQCPLGCQSVCTVREQAPEMQPPDGCSCPLYDHQAQTY
ALTTAGDTDIIFNRVATGGGKSLGATLPALLPKVHPDFRVMGLYPTIELVEDQYRQQQQYHQMF
GLNAEKRVDRLYGAELARRISEKDSNRFQELLKSIEQKPVIQTNPDIFHYITHFRYRDNARSQSEL
PMVMAKFPDLWVFDEFHIFGEHQMAAALNSLLLIRHATQSKRKFLFTSATPKTSFVESLKNSGFK
IAEIQGEYSDRPQQGYRQILQNINLEFVHLKDTDTESWLLAQSQNLRDLLHQEKNGRGLIIVNSVA
AAGHLCRNLSKILPDVEVREISGRCDRQERQQTQTYLQKSEKSVLVVATSAVDVGVDFKIHVLIC
ESSDSATVIQRLGRLGRHGGFQQYQAYVLIPSQTQWVIASLKEELEEDSSIDRLELSTVIETAFNAP
QEFKQYQKIWGELQAQGMLYQMVYGGSRSESKDNLAVTQDLRDRMIESLEKIYQKQINGFGRW
WGLSNDAVGKATQEELLRFRGGSSLQSGIWDGDRLYSYDLLKVLTYATVEVIDREIFLEAAQKL
NHGIEEFPEQYLTGYFKITKWLDQRLNFSLFSTHSHLASCQLMLLDRLQLKGHIQPELSRCLGRRK
YLAYLVPINRNNPSSHWDISRKLRLSPLFGLYRLTDSGGNAYGCAFNHDALLLEALKWRLGDFC
KQATSQSLIF
Human codon optimized Syn-ID cas3 with NLS and HA tag (SEQ ID NO: 144)
ATGAGCCTGCCAAAAGTGGGCGCCATGAAGGTGCAGGTGAAGCCCCTGTACGCCAAGCTGA
ACGAGGGACTGGGACAGTGCCCACTGGGATGTCAGAGCGTGTGCACAGTGCGGGAGCAGGC
ACCTGAGATGCAGCCACCTGACGGATGCAGCTGTCCACTGTACGATCACCAGGCCCAGACAT
ATGCCCTGACCACAGCCGGCGACACCGATATCATCTTTAATAGAGTGGCAACAGGAGGAGG
CAAGAGCCTGGGAGCCACCCTGCCAGCCCTGCTGCCAAAGGTGCACCCTGACTTCAGAGTGA
TGGGCCTGTACCCTACCATCGAGCTGGTGGAGGACCAGTACCGCCAGCAGCAGCAGTATCAC
CAGATGTTTGGCCTGAACGCCGAGAAGAGGGTGGATAGGCTGTATGGAGCAGAGCTGGCAA
GGAGAATCTCTGAGAAGGACAGCAATCGGTTCCAGGAGCTGCTGAAGTCCATCGAGCAGAA
GCCAGTGATCCAGACAAACCCCGACATCTTTCACTACATCACCCACTTCCGGTATAGAGATA
ATGCCAGATCTCAGAGCGAGCTGCCCATGGTCATGGCCAAGTTTCCTGACCTGTGGGTGTTC
GATGAGTTTCACATCTTCGGAGAGCACCAGATGGCAGCCGCCCTGAACTCCCTGCTGCTGAT
CAGGCACGCCACCCAGTCTAAGCGCAAGTTCCTGTTTACCAGCGCCACACCCAAGACCTCCT
TTGTGGAGTCTCTGAAGAATAGCGGCTTCAAGATCGCCGAGATCCAGGGCGAGTACTCTGAT
CGGCCTCAGCAGGGCTATAGACAGATCCTGCAGAACATCAATCTGGAGTTCGTGCACCTGAA
GGACACCGATACAGAGTCCTGGCTGCTGGCCCAGTCTCAGAACCTGCGGGACCTGCTGCACC
AGGAGAAGAACGGCAGAGGCCTGATCATCGTGAATTCTGTGGCAGCAGCAGGACACCTGTG
CAGGAATCTGAGCAAGATCCTGCCAGACGTGGAGGTGCGCGAGATCTCCGGCCGGTGCGAT
AGACAGGAGAGGCAGCAGACCCAGACATACCTGCAGAAGAGCGAGAAGTCCGTGCTGGTG
GTGGCCACAAGCGCCGTGGACGTGGGCGTGGATTTCAAGATCCACGTGCTGATCTGTGAGAG
CTCCGATTCCGCCACCGTGATCCAGCGCCTGGGCCGGCTGGGCAGACACGGAGGCTTTCAGC
AGTACCAGGCCTATGTGCTGATCCCATCCCAGACCCAGTGGGTCATCGCCTCTCTGAAGGAG
GAGCTGGAGGAGGACTCTAGCATCGATAGGCTGGAGCTGAGCACAGTGATCGAGACCGCCT
TTAACGCCCCCCAGGAGTTCAAGCAGTACCAGAAGATCTGGGGAGAGCTGCAGGCACAGGG
AATGCTGTACCAGATGGTGTATGGCGGCTCCAGGTCTGAGAGCAAGGATAATCTGGCCGTGA
CACAGGACCTGAGGGATCGCATGATCGAGTCCCTGGAGAAGATCTATCAGAAGCAGATCAA
CGGCTTTGGCCGCTGGTGGGGCCTGTCTAATGACGCAGTGGGCAAGGCAACCCAGGAGGAG
CTGCTGCGGTTCAGAGGCGGCTCCTCTCTGCAGAGCGGCATCTGGGACGGCGATCGGCTGTA
CTCCTATGATCTGCTGAAGGTGCTGACATACGCCACCGTGGAAGTGATCGACAGAGAGATCT
TCCTGGAGGCCGCCCAGAAGCTGAACCACGGCATCGAGGAGTTTCCCGAGCAGTACCTGAC
AGGCTATTTCAAGATCACCAAGTGGCTGGATCAGAGGCTGAATTTTTCTCTGTTCAGCACCC
ACTCCCACCTGGCCTCTTGTCAGCTGATGCTGCTGGACCGGCTGCAGCTGAAGGGCCACATC
CAGCCTGAGCTGAGCAGATGCCTGGGCAGGCGCAAGTACCTGGCCTATCTGGTGCCTATCAA
CAGGAACAATCCAAGCTCCCACTGGGACATCAGCAGGAAGCTGCGCCTGTCCCCACTGTTTG
GACTGTACAGGCTGACAGACTCCGGAGGAAACGCCTATGGCTGTGCCTTCAATCACGATGCA
CTGCTGCTGGAGGCCCTGAAGTGGAGGCTGGGCGACTTTTGCAAGCAGGCCACCTCCCAGTC
TCTGATCTTCGGCAGCGTCGGCTACCCCTACGATGTGCCTGATTACGCCCCTAAGAAAAAGC
GGAAAGTGTGA
Syn-ID CRISPR repeat sequence (SEQ ID NO: 145)
CTTTCCTTCTACTAATCCCGGCGATCGGGACTGAAAC
Syn-ID GFP targeting guide sequence (SEQ ID NO: 146)
CGTGACCGCCGCCGGGATCACTCTCGGCATGGACG
Bha-IC csd2 protein sequence (SEQ ID NO: 147)
MTILDHKIDFAVILSVTKANPNGDPLNGNRPRQNYDGHGEISDVAIKRKIRNRLLDMEEPIFVQSD
DRKADSFKSLRDRADSNPELAKMLKAKNASVDEFAKIACQEWMDVRSFGQVFAFKGSNLSVGV
RGPVSIHTATSIDPIDIVSTQITKSVNSVTGDKRSSDTMGMKHRVDFGVYVFKGSINTQLAEKTGF
TNEDAEKIKRALITLFENDSSSARPDGSMEVHKVYWWEHSSKLGQYSSAKVHRSLKIESKTDTPK
SFDDYAVELYELDGLGVEVIDGQ
Human codon optimized Bha-IC csd2 with NLS and HA tag (SEQ ID NO: 148)
ATGGTGCCTAAGAAGAAGAGAAAGGTGTACCCATACGATGTTCCAGATTACGCTGGCAGCG
GCACCATCCTGGACCACAAGATCGACTTCGCCGTGATCCTGAGCGTGACCAAGGCCAATCCT
AACGGCGACCCTCTGAACGGCAACAGACCCAGACAGAACTACGATGGCCACGGCGAGATCA
GCGACGTGGCCATTAAGCGGAAGATCCGGAACCGGCTGCTGGACATGGAAGAACCCATCTT
CGTGCAGAGCGACGACAGAAAGGCCGACAGCTTCAAGAGCCTGAGAGACAGAGCCGACAG
CAACCCTGAGCTGGCCAAGATGCTGAAGGCCAAGAATGCCAGCGTGGACGAGTTCGCCAAG
ATCGCCTGTCAAGAGTGGATGGACGTGCGGAGCTTTGGCCAGGTGTTCGCCTTCAAGGGCAG
CAATCTGAGCGTGGGCGTTAGAGGCCCTGTGTCTATTCACACCGCCACCAGCATCGACCCCA
TCGACATCGTGTCTACCCAGATCACCAAGAGCGTGAACAGCGTGACCGGCGACAAGAGAAG
CAGCGACACCATGGGCATGAAGCACAGAGTGGACTTCGGCGTGTACGTGTTCAAGGGCTCC
ATCAACACCCAGCTGGCCGAGAAAACCGGCTTCACCAATGAGGACGCCGAGAAGATCAAGC
GGGCCCTGATCACCCTGTTCGAGAACGATAGCAGCAGCGCCAGACCTGACGGCAGCATGGA
AGTGCACAAAGTGTATTGGTGGGAGCACAGCAGCAAGCTGGGCCAGTACTCTAGCGCCAAG
GTGCACAGAAGCCTGAAGATCGAGAGCAAGACCGACACACCCAAGAGCTTCGACGACTACG
CCGTGGAACTGTACGAGCTGGATGGCCTGGGCGTCGAAGTGATCGATGGACAATAA
Bha-IC Cas5 protein sequence (SEQ ID NO: 149)
MRNEVQFELFGDYALFTDPLTKIGGEKLSYSVPTYQALKGIAESIYWKPTIVFVIDELRVMKPIQM
ESKGVRPIEYGGGNTLAHYTYLKDVHYQVKAHFEFNLHRPDLAFDRNEGKHYSILQRSLKAGGR
RDIFLGARECQGYVAPCEFGSGDGFYDGQGKYHLGTMVHGFNYPDETGQHQLDVRLWSAVME
NGYIQFPRPEDCPIVRPVKEMEPKIFNPDNVQSAEQLLHDLGGE
Human codon optimized Bha-IC cas5 with NLS and HA tag (SEQ ID NO: 150)
ATGAGAAACGAGGTGCAGTTCGAGCTGTTCGGCGACTACGCCCTGTTCACCGATCCTCTGAC
AAAGATCGGCGGCGAGAAGCTGAGCTACAGCGTGCCAACATATCAGGCCCTGAAGGGAATC
GCCGAGAGCATCTACTGGAAGCCCACCATCGTGTTCGTGATCGACGAGCTGAGAGTGATGA
AGCCCATCCAGATGGAAAGCAAAGGCGTGCGGCCCATCGAGTACGGCGGAGGAAATACTCT
GGCCCACTACACCTACCTGAAGGACGTGCACTACCAAGTGAAGGCCCACTTCGAGTTCAACC
TGCACAGACCCGACCTGGCCTTCGACAGAAATGAGGGCAAGCACTACAGCATCCTGCAGCG
GAGTCTGAAGGCTGGCGGCAGAAGAGACATCTTCCTGGGCGCTAGAGAATGCCAGGGCTAT
GTGGCCCCTTGCGAGTTTGGAAGCGGCGACGGCTTTTATGACGGCCAGGGCAAGTATCACCT
GGGCACAATGGTGCACGGCTTCAACTACCCCGATGAGACAGGACAGCACCAGCTGGATGTT
CGGCTTTGGAGCGCCGTGATGGAAAACGGCTACATTCAGTTCCCCAGACCTGAGGACTGCCC
CATTGTGCGGCCTGTGAAAGAGATGGAACCCAAGATCTTCAACCCCGACAACGTGCAGTCTG
CCGAGCAGCTCCTGCATGATCTTGGCGGAGAATACCCATACGATGTTCCAGATTACGCTGTG
CCTAAGAAGAAGAGAAAGGTGTAA
Bha-IC csd1 protein sequence (SEQ ID NO: 151)
MSWLLHLYETYEANLDQVGKTVKKGEDREYTLLPISHTTQNAHIEVTLDEDGDFLRAKALTKES
TLIPCTEEAASRSGSKVAPYPLHDKLSYVAGDFVKYGGKIKNQDDAPFDTYIKNLGEWANSPYA
TEKVKCIYTYLKKGRLIEDLVDAGVLKLDENQQLIEKWEKRYEELLGEKPAIFSSGATDQASAFV
RFNVFHPESIDDVWKDKEMFDSFISFYNDKLGEEDICFVTGNRLPSTERHANKIRHAADKAKLISA
NDNSGFTFRGRFKTSREAVGISYEVSQKAHNALKWLIHRQSKSIDDRVFLVWSNDNSLVPNPDE
DAVDIMKHANRELERDPDTGQIFAGEVKKAIGGYRSDLNYQPEVHILVLDSATTGRMAVLYYRS
LNKELYLNRLEAWHDSCAWEHRYRRDEKEFISFYGAPATKDIAFAAYGPRASEKVIKDLMERML
PCIVDGRRVPKDIVRSAFQRASNPVSMERWEWEKTLSITCALIRKMHIEQKEEWGVPLDKSSTDR
SYLFGRLLAVADVLERGALGKDETRATNAIRYMNSYSKNPGRTWKTIQESLQPYQAKLGTKAT
YLSKLVDEIGDQFEPGDENNNPLTEQYLLGFYSQRRELYKKKEEETNQ
Human codon optimized Bha-IC csd1 with NLS and twin Strep tag (SEQ ID NO: 152)
ATGGTTCCCAAGAAGAAGCGGAAAGTCTGGTCCCATCCTCAGTTCGAGAAAGGCGGAGGAT
CTGGCGGTGGTTCTGGTGGATCTGCTTGGAGCCATCCACAATTTGAAAAAGGCAGCGGCATG
AGCTGGCTGCTGCACCTGTACGAGACATACGAGGCCAACCTGGACCAAGTGGGCAAGACCG
TGAAGAAGGGCGAAGATAGAGAGTACACCCTGCTGCCTATCAGCCACACCACACAGAACGC
CCACATCGAAGTGACCCTGGACGAGGACGGCGACTTCCTGAGAGCCAAGGCTCTGACCAAA
GAGAGCACACTGATCCCTTGCACCGAGGAAGCCGCCAGCAGATCTGGCTCTAAGGTGGCCC
CTTATCCTCTGCACGACAAGCTGTCTTACGTGGCCGGCGATTTCGTGAAGTACGGCGGCAAG
ATCAAGAACCAGGACGACGCCCCTTTCGACACCTACATCAAGAATCTCGGCGAGTGGGCCA
ACTCTCCCTACGCCACAGAGAAAGTGAAGTGCATCTACACCTACCTGAAGAAAGGCCGGCT
GATCGAGGACCTGGTGGATGCTGGTGTCCTGAAGCTGGACGAGAACCAGCAGCTGATTGAG
AAGTGGGAGAAGCGCTACGAGGAACTGCTGGGAGAGAAGCCCGCCATCTTTAGCTCTGGCG
CCACAGATCAGGCCAGCGCCTTCGTGCGGTTCAATGTGTTTCACCCCGAGAGCATCGACGAC
GTGTGGAAGGACAAAGAGATGTTCGACAGCTTCATCAGCTTCTACAACGATAAGCTGGGCG
AAGAGGACATCTGCTTCGTGACCGGCAACAGACTGCCCAGCACAGAGAGACACGCCAACAA
GATTAGACACGCCGCCGACAAGGCCAAGCTGATCTCCGCCAATGACAACAGCGGCTTCACCT
TCCGGGGCAGATTCAAGACCAGCAGAGAAGCCGTGGGCATCAGCTACGAGGTGTCCCAGAA
AGCCCACAACGCCCTGAAGTGGCTGATCCACAGACAGAGCAAGTCTATCGACGACCGGGTG
TTCCTCGTGTGGTCCAACGACAATAGCCTGGTGCCTAATCCTGACGAGGATGCCGTGGACAT
CATGAAGCACGCCAATAGAGAGCTGGAACGGGACCCTGATACCGGCCAGATTTTTGCCGGC
GAAGTGAAGAAAGCCATCGGCGGCTACAGAAGCGACCTGAACTATCAGCCCGAGGTGCACA
TCCTGGTGCTGGATTCTGCCACCACCGGAAGAATGGCTGTGCTGTACTACCGCAGCCTGAAC
AAAGAGCTGTACCTGAACCGGCTGGAAGCCTGGCACGATTCTTGTGCCTGGGAGCACAGAT
ACCGGCGGGACGAGAAAGAGTTCATCTCCTTCTACGGCGCTCCCGCCACCAAGGATATTGCC
TTTGCCGCCTATGGACCCAGAGCCAGCGAAAAAGTGATCAAGGATCTGATGGAACGGATGC
TGCCCTGCATCGTGGACGGCAGAAGAGTGCCTAAGGACATTGTGCGGAGCGCCTTCCAGAG
GGCCAGCAATCCTGTGTCCATGGAAAGATGGGAGTGGGAAAAGACCCTGAGCATCACATGC
GCCCTGATCCGGAAGATGCACATCGAGCAGAAAGAGGAATGGGGCGTCCCACTGGACAAGA
GCAGCACCGATAGAAGCTACCTGTTCGGCAGACTGCTGGCCGTGGCTGACGTTTTGGAAAGA
GGCGCCCTGGGCAAAGACGAGACAAGAGCCACAAACGCCATCCGGTACATGAACAGCTACA
GCAAGAACCCCGGCAGAACCTGGAAAACCATCCAAGAGAGCCTGCAGCCTTACCAGGCCAA
ACTGGGCACCAAGGCCACATACCTGAGCAAGCTGGTGGACGAGATCGGGGACCAGTTTGAG
CCCGGCGACTTTAACAACAACCCTCTGACCGAGCAGTACCTGCTGGGCTTCTACAGCCAGCG
GCGGGAACTGTACAAGAAGAAAGAAGAGGAAACGAACCAGTAA
Bha-IC Cas11 protein sequence (SEQ ID NO: 153)
MPLDKSSTDRSYLFGRLLAVADVLERGALGKDETRATNAIRYMNSYSKNPGRTWKTIQESLQPY
QAKLGTKATYLSKLVDEIGDQFEPGDFNNNPLTEQYLLGFYSQRRELYKKKEEETNQ
Human codon optimized Bha-IC cas11 (SEQ ID NO: 154)
ATGCCACTGGACAAGAGCAGCACCGATAGAAGCTACCTGTTCGGCAGACTGCTGGCCGTGG
CTGACGTTTTGGAAAGAGGCGCCCTGGGCAAAGACGAGACAAGAGCCACAAACGCCATCCG
GTACATGAACAGCTACAGCAAGAACCCCGGCAGAACCTGGAAAACCATCCAAGAGAGCCTG
CAGCCTTACCAGGCCAAACTGGGCACCAAGGCCACATACCTGAGCAAGCTGGTGGACGAGA
TCGGGGACCAGTTTGAGCCCGGCGACTTTAACAACAACCCTCTGACCGAGCAGTACCTGCTG
GGCTTCTACAGCCAGCGGCGGGAACTGTACAAGAAGAAAGAAGAGGAAACGAACCAGTAA
Bha-IC Cas3 protein sequence (SEQ ID NO: 155)
MYIAHIREVDKVIQTLKEHLCGVQCLAETFGAKLRLQHVAGLAGLLHDLGKYTNEFKDYIYKAV
FEPELAEKKRGQVDHSTAGGRLLYQMLHDRENSFHEKLLAEVVGNAIISHHSNLQDYISPTIESNF
LTRVLEKELPEYESAVERFFQEVMTEAELARYVAKAVDEIKQFTDNSPTQSFFLTKYIFSCLIDAD
RTNTRMFDEQAREEEPTQPQQLFEHYHQQLLNHLASLKESDSAQKPINVLRSAMSEQCESFAMR
PSGIYTLSIPTGGGKTLASLRYALKHAQEYNKQRIIYIVPFTTIIEQNAQEVRNILGDDENILEHHSN
VVEDSENGDEQEDGVITKKERLRLARDNWDRPIIFTTLVQFLNVFYAKGNRNTRRLHNLSHSVLI
FDEVQKVPTKCVSLFNEALNFLKEFAHCSILLCTATQPTLENVKHSLLKDRDGEIVQNLTEVSEAF
KRVEILDKTDQPMTNERLAEWVRDEAPSWGSTLIILNTKKVVKDLYEKLEGGPLPVFHLSTSMC
AAHRKDQLDEIRALLKEGTPFICVTTQLIEAGVDVSFKCVIRSLAGLDSIAQAAGRCNRHGEEQL
QYVYVIDHABETLSKLKEIEVGQEIAGNVLARFKKKAEKYEGNLLSQAAMREYFRYYYSKMDA
NLNYFVKEVDKDMTKLLMSHAVENSYVTYYQKNTGTHFPLLLNGSYKTAADHERVIDQNTTSA
IVPYGEGQDIIAQLNSGEWVDDLSKVLKKAQQYTVNLYSQEIDQLKKEGAIVMHLDGMVYELK
ESWYSHQYGVDFKGEGGMDFMSF
Human codon optimized Bha-IC cas3 with NLS and twin Strep tag (SEQ ID NO: 156)
ATGTATATCGCCCACATCCGCGAGGTGGACAAAGTGATCCAGACACTGAAAGAACACCTGT
GCGGCGTGCAGTGCCTGGCCGAAACATTTGGAGCCAAGCTGAGACTGCAGCACGTGGCAGG
ACTGGCTGGACTGCTGCATGATCTGGGCAAGTACACCAACGAGTTCAAGGACTACATCTACA
AGGCCGTGTTCGAGCCCGAGCTGGCCGAGAAAAAGAGAGGCCAGGTCGACCACTCTACCGC
TGGTGGCAGACTGCTGTACCAGATGCTGCACGACAGAGAGAACAGCTTCCACGAGAAGCTG
CTGGCCGAAGTCGTGGGCAATGCCATCATCAGCCACCACAGCAACCTGCAGGATTACATCAG
CCCTACAATCGAGAGCAACTTCCTGACCAGAGTGCTGGAAAAAGAGCTGCCCGAGTACGAG
AGCGCCGTGGAACGGTTCTTCCAAGAAGTGATGACCGAGGCCGAACTGGCCAGATACGTGG
CCAAAGCCGTGGACGAGATCAAGCAGTTCACCGACAACAGCCCTACTCAGTCATTCTTTCTG
ACCAAGTACATCTTCAGCTGCCTGATCGACGCCGACCGGACCAACACCAGAATGTTCGATGA
GCAGGCCAGAGAGGAAGAACCCACACAGCCTCAGCAGCTGTTCGAGCACTATCACCAGCAA
CTGCTGAACCACCTGGCCAGCCTGAAAGAGAGCGACAGCGCCCAGAAACCTATCAACGTGC
TGAGAAGCGCCATGAGCGAGCAGTGCGAGAGCTTTGCCATGAGGCCTAGCGGCATCTACAC
CCTGTCTATCCCAACAGGCGGCGGAAAGACCCTGGCTTCTCTGAGATATGCCCTGAAGCACG
CCCAAGAGTACAACAAGCAGCGGATCATCTACATCGTGCCCTTCACCACCATCATCGAGCAG
AACGCTCAAGAAGTGCGGAACATCCTGGGCGACGACGAGAATATCCTGGAACACCACTCCA
ACGTGGTGGAAGATAGCGAGAACGGCGACGAGCAAGAGGACGGCGTGATCACCAAGAAAG
AGAGACTGAGACTGGCCCGGGACAACTGGGACAGACCCATCATCTTTACAACCCTGGTGCA
GTTCCTGAACGTGTTCTACGCCAAGGGCAACAGAAACACCAGACGGCTGCACAACCTGAGC
CACAGCGTGCTGATCTTCGACGAGGTGCAGAAAGTGCCCACCAAATGCGTGTCCCTGTTCAA
CGAGGCCCTGAACTTTCTGAAAGAGTTCGCCCACTGCAGCATCCTGCTGTGCACTGCCACTC
AGCCCACACTGGAAAACGTGAAGCACAGCCTGCTGAAGGACCGCGACGGCGAGATTGTGCA
GAACCTGACCGAAGTGTCCGAGGCCTTCAAGAGAGTGGAAATCCTGGACAAGACCGACCAG
CCTATGACCAACGAAAGACTGGCCGAGTGGGTCCGAGATGAGGCTCCTTCTTGGGGCTCTAC
CCTGATCATCCTGAATACCAAGAAGGTGGTCAAGGACCTGTATGAGAAGCTGGAAGGGGC
CCTCTGCCTGTGTTTCACCTGAGCACCTCTATGTGCGCCGCTCACAGAAAGGACCAGCTGGA
TGAGATCAGAGCCCTGCTGAAAGAGGGCACCCCTTTCATCTGCGTGACCACACAGCTGATTG
AGGCCGGCGTGGACGTGTCCTTTAAGTGCGTGATCAGAAGCCTGGCCGGCCTGGATTCTATT
GCCCAGGCTGCCGGAAGATGCAACAGACACGGCGAAGAACAGCTCCAGTACGTGTACGTGA
TCGACCACGCCGAGGAAACCCTGAGCAAGCTGAAAGAAATCGAAGTGGGCCAAGAGATCGC
CGGCAATGTGCTGGCCCGGTTCAAGAAGAAGGCCGAGAAGTACGAGGGCAACCTGCTGTCT
CAGGCCGCCATGAGAGAGTACTTCCGGTACTACTACAGCAAGATGGACGCCAACCTGAACT
ACTTCGTGAAAGAAGTCGACAAGGACATGACCAAGCTGCTGATGAGCCACGCCGTCGAGAA
CTCCTACGTGACCTACTACCAGAAGAACACCGGCACACACTTCCCTCTGCTGCTGAACGGCA
GCTACAAGACAGCCGCCGACCACTTCAGAGTGATTGACCAGAATACCACCAGCGCCATCGT
GCCTTATGGCGAAGGCCAGGACATCATTGCCCAGCTGAATAGCGGCGAGTGGGTTGACGAT
CTGAGCAAGGTGCTGAAGAAAGCCCAGCAGTACACCGTGAACCTGTACTCCCAAGAGATTG
ATCAGCTGAAAAAAGAGGGCGCCATTGTGATGCACCTGGACGGCATGGTGTACGAACTCAA
AGAGAGCTGGTACTCCCACCAGTATGGCGTGGACTTCAAAGGCGAAGGCGGCATGGACTTC
ATGAGCTTCGGCAGCGGCTGGTCCCATCCTCAGTTCGAGAAAGGCGGAGGATCTGGCGGTG
GTTCTGGTGGATCTGCTTGGAGCCATCCACAATTTGAAAAAGTGCCTAAGAAGAAGAGAAA
GGTGTAA
Bha-IC CRISPR repeat sequence (SEQ ID NO: 157)
TCGCACTCTTCATGGGTGCGTGGATTGAAAT
Bha-IC GFP targeting sequence (SEQ ID NO: 158)
GAGGGCGACACCCTGGTGAACCGCATCGAGCTGAAG
Dvu-IC Cas5 protein sequence (SEQ ID NO: 159)
MTHGAVKTYGIRLRVWGDYACFTRPEMKVERVSYDVMPPSAARGILEAIHWKPAIRWIVDRIHV
LRPIVFDNVRRNEVSSKIPKPNPATAMRDRKPLYFLVDDGSNRQQRAATLLRNVDYVIEAHFELT
DKAGAEDNAGKHLDIFRRRARAGQSFQQPCLGCREFPASFELLEGDVPLSCYAGEKRDLGYMLL
DIDFERDMTPLFFKAVMEDGVITPPSRTSPEVRA
Human codon optimized Dvu-IC cas5 with NLS and HA tag (SEQ ID NO: 160)
ATGACACACGGGGCCGTGAAAACCTACGGCATCAGACTGAGAGTGTGGGGCGACTACGCCT
GCTTCACCAGACCTGAGATGAAGGTGGAACGGGTGTCCTACGACGTGATGCCTCCATCTGCC
GCCAGAGGAATCCTGGAAGCCATCCACTGGAAGCCCGCCATCAGATGGATCGTGGACAGAA
TCCACGTGCTGCGGCCCATCGTGTTCGACAACGTGCGGAGAAATGAGGTGTCCAGCAAGATC
CCCAAGCCTAATCCTGCCACCGCCATGAGAGACAGAAAGCCCCTGTACTTCCTGGTGGACGA
CGGCAGCAACAGACAGCAGAGAGCTGCCACACTGCTGCGGAACGTGGACTATGTGATCGAG
GCCCACTTCGAGCTGACCGATAAGGCTGGCGCCGAGGATAATGCCGGCAAGCACCTGGACA
TCTTCCGGCGGAGAGCTAGAGCCGGCCAGTCTTTTCAGCAGCCTTGCCTGGGCTGCAGAGAG
TTCCCTGCCTCTTTCGAACTGCTGGAAGGCGACGTGCCCCTGTCTTGTTATGCCGGCGAGAAG
CGCGATCTGGGCTACATGCTGCTGGACATCGACTTCGAGCGGGACATGACCCCTCTGTTCTT
CAAGGCCGTGATGGAAGATGGCGTGATCACCCCTCCTAGCCGGACATCTCCTGAAGTTCGAG
CTGGCAGCGTCGGCTACCCCTACGATGTGCCTGATTACGCCCCTAAGAAAAAGCGGAAAGTG
TGA
Dvu-IC Cas8 protein sequence (SEQ ID NO: 161)
MILQALHGYYQRMSADPDAGMPPYGTSMENISFALVLDAKGTLRGIEDLREQEGKKLRPRKML
VPIAEKKGNGIKPNFLWENTSYILGVDAKGKQERTDKCHAAFIAHIKAYCDTADQDLAAVLQFL
EHGEKDLSAFPVSEEVIGSNIVFRIEGEPGFVHERPAARQAWANCLNRREQGLCGQCLITGERQK
PIAQLHPSIKGGRDGVRGAQAVASIVSFNNTAFESYGKEQSINAPVSQEAAFSYVTALNYLLNPSN
RQKVTIADATVVFWAERSSPAEDIFAGMEDPPSTTAKPESSNGTPPEDSEEGSQPDTARDDPHAA
ARMHDLLVAIRSGKRATDIMPDMDESVRFHVLGLSPNAARLSVRFWEVDTVGHMLDKVGRHY
RELEIPQFNNEQEFPSLSTLLRQTAVLNKTENISPVLAGGLFRAMLTGGPYPQSLLPAVLGRIRAE
HARPEDKSRYRLEVVTYYRAALIKAYLIRNRKLEVPVSLDPARTDRPYLLGRLFAVLEKAQEDA
VPGANATIKDRYLASASANPGQVFHMLLKNASNHTAKLRKDPERKGSAIHYEIMMQEIIDNISDF
PVTMSSDEQGLFMIGYYHQRKALFTKKNKEN
Human codon optimized Dvu-IC cas8 with NLS and HA tag (SEQ ID NO: 162)
ATGGTGCCCAAGAAAAAGCGGAAGGTGTACCCCTACGACGTGCCCGATTATGCCGGCTCTGT
GGGAATTCTGCAGGCCCTGCACGGCTACTACCAGAGAATGAGCGCCGATCCTGACGCCGGC
ATGCCTCCTTATGGCACCAGCATGGAAAACATCAGCTTCGCCCTGGTGCTGGACGCCAAGGG
AACACTGAGAGGCATCGAGGACCTGAGAGAGCAAGAGGGCAAGAAGCTGCGGCCCAGAAA
GATGCTGGTTCCTATCGCCGAGAAGAAAGGCAACGGAATCAAGCCCAACTTCCTGTGGGAG
AACACCAGCTACATCCTCGGCGTGGACGCTAAGGGCAAGCAAGAGCGGACCGATAAGTGCC
ACGCCGCCTTTATCGCCCACATCAAGGCCTACTGCGACACCGCCGATCAGGATCTGGCTGCC
GTGCTGCAGTTTCTGGAACACGGCGAGAAGGATCTGAGCGCCTTTCCTGTGTCCGAGGAAGT
GATCGGCAGCAACATCGTGTTCCGGATCGAGGGCGAGCCTGGCTTTGTGCACGAAAGACCTG
CTGCCAGACAGGCCTGGGCCAACTGCCTGAATAGAAGAGAACAGGGCCTGTGCGGCCAGTG
CCTGATTACAGGCGAGAGACAGAAGCCTATCGCTCAGCTGCACCCCAGCATCAAAGGCGGA
AGAGATGGCGTTAGAGGCGCCCAGGCTGTGGCCAGCATCGTGTCCTTTAACAACACCGCCTT
CGAGAGCTACGGCAAAGAGCAGAGCATCAACGCCCCTGTGTCTCAAGAGGCCGCCTTCAGC
TATGTGACAGCCCTGAACTACCTGCTGAACCCCTCCAACCGGCAGAAAGTGACAATCGCCGA
TGCCACCGTGGTGTTTTGGGCCGAGAGATCTAGCCCTGCCGAGGATATCTTTGCCGGCATGT
TCGACCCTCCAAGCACCACAGCCAAGCCAGAGTCCAGCAATGGCACCCCTCCTGAGGATAG
CGAGGAAGGCTCTCAGCCTGACACCGCCAGAGATGATCCTCATGCCGCCGCTAGAATGCAC
GATCTGCTGGTGGCCATCAGATCTGGCAAACGGGCCACCGACATCATGCCCGACATGGATGA
GAGCGTGCGGTTCCATGTGCTGGGACTGTCTCCAAATGCCGCCAGACTGTCCGTGCGCTTCT
GGGAAGTTGATACCGTGGGCCACATGCTGGACAAAGTGGGCAGACACTACAGAGAGCTGGA
AATCATCCCTCAGTTCAACAACGAGCAAGAGTTCCCCAGCCTGAGCACCCTGCTGAGACAGA
CAGCCGTGCTGAACAAGACCGAGAACATCAGCCCAGTGCTGGCTGGCGGACTGTTCAGAGC
TATGCTTACCGGCGGACCCTATCCACAGTCTCTGCTGCCTGCTGTGCTGGGCAGAATTAGAG
CCGAACACGCCAGACCAGAGGACAAGAGCCGGTACAGACTGGAAGTGGTCACCTACTACCG
GGCTGCCCTGATTAAGGCCTACCTGATCAGAAACCGGAAGCTGGAAGTGCCCGTGTCTCTGG
ATCCTGCCAGAACCGACAGACCTTACCTGCTGGGGAGACTGTTCGCCGTGCTGGAAAAGGCC
CAAGAGGATGCTGTGCCTGGCGCCAATGCCACCATCAAGGATAGATACCTGGCCAGCGCCTC
CGCCAATCCTGGACAGGTTTTCCATATGCTGCTGAAGAACGCCAGCAACCACACCGCCAAGC
TGAGAAAGGACCCCGAGAGAAAGGGCAGCGCCATCCACTACGAGATCATGATGCAAGAGAT
CATCGACAACATCAGCGACTTCCCCGTGACCATGAGCAGCGACGAGCAGGGGCTGTTCATG
ATCGGCTACTATCACCAGCGGAAAGCCCTGTTCACCAAGAAGAACAAAGAGAACTAG
Dvu-IC Cas7 protein sequence (SEQ ID NO: 163)
MTAIANRYEFVLLFDVENGNPNGDPDAGNMPRIDPETGHGLVTDVCLKRKIRNHVALTKEGAER
FNIYIQEKAILNETHERAYTACDLKPEPKKLPKKVEDAKRVTDWMCTNFYDIRTFGAVMTTEVN
CGQVRGPVQMAFARSVEPVVPQEVSITRMAVTTKAEAEKQQGDNRTMGRKHIVPYGLYVAHGF
ISAPLAEKTGFSDEDLTLFWDALVNMFEHDRSAARGLMSSRKLIVFKHQNRLGNAPAHKLFDLV
KVSRAEGSSGPARSFADYAVTVGQAPEGVEVKEML
Human codon optimized Dva-IC cas7 with NLS and HA tag (SEQ ID NO: 164)
ATGACCGCCATTGCCAACAGATACGAGTTCGTGCTGCTGTTCGACGTGGAAAACGGCAACCC
CAACGGCGATCCTGACGCCGGCAATATGCCCAGAATCGACCCTGAGACAGGCCACGGCCTG
GTCACAGATGTGTGCCTGAAGCGGAAGATCCGGAACCACGTGGCCCTGACAAAAGAGGGCG
CCGAGCGGTTCAACATCTACATCCAAGAGAAGGCCATCCTGAACGAGACACACGAGAGAGC
CTACACCGCCTGCGATCTGAAGCCCGAGCCTAAGAAACTGCCCAAGAAGGTCGAGGACGCC
AAGCGCGTGACCGATTGGATGTGCACCAACTTCTACGACATCCGGACCTTCGGCGCCGTGAT
GACCACCGAAGTGAATTGTGGACAAGTGCGGGGACCCGTGCAGATGGCCTTTGCCAGATCT
GTGGAACCCGTGGTGCCCCAAGAGGTGTCCATCACAAGAATGGCCGTGACCACAAAGGCCG
AGGCCGAAAAACAGCAGGGCGACAACAGAACCATGGGCAGAAAGCACATCGTGCCCTACG
GCCTGTATGTGGCCCACGGCTTTATTTCTGCCCCTCTGGCCGAGAAAACCGGCTTCTCCGATG
AGGATCTGACCCTGTTCTGGGACGCCCTGGTCAACATGTTCGAGCACGATAGATCTGCCGCC
AGAGGCCTGATGAGCAGCAGAAAGCTGATCGTGTTCAAGCACCAGAACCGGCTGGGCAATG
CCCCTGCTCACAAGCTGTTCGATCTGGTCAAGGTGTCCAGAGCCGAGGGCAGTTCTGGACCT
GCCAGAAGCTTTGCCGATTACGCCGTGACAGTTGGACAGGCCCCTGAAGGCGTGGAAGTGA
AAGAGATGCTGGGCTCTGTGGGCTATCCTTACGACGTGCCAGACTACGCCCCTAAGAAAAAG
CGCAAAGTGTGA
Dvu-IC Cas11 protein sequence (SEQ ID NO: 165)
MSLDPARTDRPYLLGRLFAVLEKAQEDAVPGANATIKDRYLASASANPGQVFHMLLKNASNHT
AKLRKDPERKGSAIHYEIMMQEIIDNISDFPVTMSSDEQGLFMIGYYHQRKALFTKKNKEN
Human codon optimized Dvu-IC cas11 (SEQ ID NO: 166)
ATGTCTCTGGATCCTGCCAGAACCGACAGACCTTACCTGCTGGGGAGACTGTTCGCCGTGCT
GGAAAAGGCCCAAGAGGATGCTGTGCCTGGCGCCAATGCCACCATCAAGGATAGATACCTG
GCCAGCGCCTCCGCCAATCCTGGACAGGTTTTCCATATGCTGCTGAAGAACGCCAGCAACCA
CACCGCCAAGCTGAGAAAGGACCCCGAGAGAAAGGGCAGCGCCATCCACTACGAGATCATG
ATGCAAGAGATCATCGACAACATCAGCGACTTCCCCGTGACCATGAGCAGCGACGAGCAGG
GGCTGTTCATGATCGGCTACTATCACCAGCGGAAAGCCCTGTTCACCAAGAAGAACAAAGA
GAACTAA
Dvu-IC Cas3 protein sequence (SEQ ID NO: 167)
MADGGDEHASGHDNTLKNARYYAHSTPNPDKSDWQGLDAHLENVANLAATFAEAFGAREWG
KAAGLLHDAGKATAQFTQRLEGRPVRVNHSICGARLAQEQGSTCGLLLSYAIAGHHGGLPDGGL
QDGQLHHRLKHERLPADVSPPSVDIRPDVLKPPFTCRPEHPGFSLSFFTRMLFSCLTDADFLDTEA
FCTPEKASARNGRSALGLVALRDALNTHLDTVERKALPSRVNDIRKTVLHDCRARASETPGLFSL
TVPTGGGKTLSSMAFALDHAVTHGLRRVIYAIPFTSIIEQNAKVFSDVFGQDNVLEHHCNYRSKD
EPEEQGYDKWRGLAAENWDAPVVVTTNVQFFESLFSNRPSRCRKLHNIARSVIVLDEAQAIPTEY
LEPCLYALKELVGQYGCTVVLCTATQPAVDDASLPERVRLHHVREIIADPQRLYTDLKRTEVTLA
GRLTDAALAARLDGHGQVLCIVGTKPQAQAVESLLQEREGAFHLSTNMYPEHRRRVLGTIRQRL
ADRLPCRVVSTSLIEAGVDVDFPVVYRAMAGLDSIAQAAGRCNREGRLPEPGQVVVYEPEKPAR
MPWMQRCASRAQETLRTLPEADPLGLEAIRRYFGLIYDVQELDRKDIFKRLRGQVDRDMVFKFR
EIANDFRFIDDEGTALVIPTGPEVEDLVRRLRGCEFPRPVLRKLQQYSVTVRHRELEKLRSAGAVE
MIGDAYPVLRNLAAYSEDMGLCVDSVEVWQPEGLVS
Human codon optimized Dvu-IC cas3 with NLS and HA tag (SEQ ID NO: 168)
ATGGCTGATGGCGGAGATGAACACGCCAGCGGCCACGACAACACCCTGAAGAACGCCAGAT
ATTACGCCCACAGCACCCCTAATCCTGACAAGAGCGATTGGCAGGGCCTCGACGCCCACCTG
GAAAATGTGGCTAATCTGGCCGCCACCTTCGCCGAAGCCTTTGGAGCTAGAGAGTGGGGAA
AAGCCGCCGGACTGCTGCACGATGCCGGAAAAGCTACAGCCCAGTTCACCCAGAGACTGGA
AGGCAGACCCGTCAGAGTGAACCACTCTATCTGTGGCGCCAGACTGGCCCAAGAGCAGGGC
TCTACTTGTGGCCTGCTGCTGAGCTATGCCATTGCCGGACATCATGGCGGACTGCCAGATGG
TGGACTGCAGGATGGACAGCTGCACCACAGACTGAAGCACGAGAGACTGCCCGCCGATGTG
TCTCCTCCTAGCGTGGACATCAGACCCGACGTGCTGAAGCCTCCTTTCACCTGTCGGCCTGAG
CACCCTGGCTTCAGCCTGAGCTTCTTCACCCGGATGCTGTTCAGCTGCCTGACCGACGCCGAT
TTCCTGGATACCGAGGCCTTCTGCACCCCTGAGAAAGCCTCTGCCAGAAATGGGAGATCTGC
CCTGGGACTCGTGGCCCTGAGAGATGCCCTGAACACACACCTGGACACCGTGGAAAGAAAA
GCCCTGCCTAGCCGCGTGAACGACATCAGAAAGACCGTGCTGCATGACTGCAGAGCCAGAG
CCTCTGAAACCCCTGGCCTGTTCTCTCTGACAGTGCCTACAGGCGGCGGAAAGACCCTGAGC
AGCATGGCCTTTGCTCTGGACCACGCCGTGACACACGGACTGAGAAGAGTGATCTACGCTAT
CCCCTTCACCAGCATCATCGAGCAGAACGCCAAGGTGTTCAGCGACGTGTTCGGCCAGGACA
ACGTGCTGGAACACCACTGCAACTACAGAAGCAAGGACGAGCCCGAGGAACAGGGCTACGA
TAAGTGGCGAGGACTGGCCGCTGAGAACTGGGATGCTCCTGTGGTCGTGACCACCAACGTGC
AGTTCTTCGAGAGCCTGTTCAGCAACAGACCCAGCCGGTGCCGGAAGCTGCACAATATCGCC
AGATCCGTGATCGTGCTGGACGAGGCCCAGGCCATTCCTACCGAGTACCTGGAACCTTGCCT
GTACGCCCTGAAAGAACTCGTGGGCCAGTACGGCTGTACCGTGGTGCTGTGTACAGCCACAC
AGCCCGCTGTGGATGATGCCTCTCTGCCTGAGAGAGTGCGGCTGCATCACGTGCGCGAGATC
ATTGCCGATCCTCAGAGACTGTACACCGACCTGAAGCGGACCGAAGTGACACTGGCTGGCA
GACTGACAGATGCCGCTCTGGCTGCTAGACTGGATGGACATGGCCAGGTGCTGTGCATCGTG
GGCACAAAACCTCAGGCACAGGCCGTGTTCAGCCTGCTGCAAGAAAGAGAGGGCGCCTTCC
ACCTGTCCACCAACATGTACCCCGAACACAGGCGGAGAGTGCTGGGCACCATTAGACAGAG
GCTGGCCGATAGACTGCCCTGCAGAGTGGTGTCTACCAGCCTGATTGAAGCCGGCGTGGACG
TGGACTTCCCCGTGGTGTATAGAGCTATGGCCGGCCTGGATTCTATCGCCCAGGCTGCCGGA
CGGTGCAATAGAGAGGGTAGACTGCCTGAGCCTGGACAGGTCGTGGTGTACGAGCCTGAGA
AGCCAGCCAGAATGCCCTGGATGCAGAGATGTGCCAGCAGAGCCCAAGAGACACTGAGAAC
CCTGCCTGAGGCTGATCCTCTCGGACTGGAAGCCATCAGACGGTACTTCGGCCTGATCTACG
ACGTGCAAGAGCTGGACCGGAAGGACATCTTCAAGCGGCTGAGAGGCCAGGTGGACAGAGA
CATGGTGTTCAAGTTCAGAGAGATCGCCAACGACTTCCGGTTCATCGACGATGAGGGCACAG
CCCTGGTCATCCCAACAGGACCTGAGGTGGAAGATCTCGTGCGGAGACTGAGAGGCTGCGA
GTTCCCTAGACCTGTGCTGCGGAAACTGCAGCAGTACAGCGTGACAGTGCGGCACAGAGAG
CTGGAAAAGCTGAGATCTGCTGGCGCCGTGGAAATGATCGGCGACGCTTATCCCGTGCTGAG
AAACCTGGCCGCCTACAGCGAAGATATGGGCCTGTGTGTGGATAGCGTGGAAGTGTGGCAG
CCTGAAGGCCTGGTTTCTCTGGGCTCTGTGGGCTACCCCTACGATGTGCCTGATTACGCCGGC
AGCTACCCTGAGTTCCCCAAGAAAAAGCGGAAAGTGTGA
Dvu-IC CRISPR repeat sequence (SEQ ID NO: 169)
GTCGCCCCCCACGCGGGGGCGTGGATTGAAAC
Dvu-IC GFP targeting guide sequence (SEQ ID NO: 170)
AGGGTCAGCTTGCCGTAGGTGGCATCGCCCTCGCC

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A system for altering a target nucleic acid sequence comprising:
an engineered Clustered Regularly Interspaced Short Palindromic Repeats (CRISPR)-CRISPR associated (Cas) (CRISPR-Cas) system, and/or one or more nucleic acids encoding the engineered CRISPR-Cas system, wherein the engineered CRISPR-Cas system comprises:
(a) Cas11;
(b) Cas3;
(c) two or more additional Cas proteins from a CRISPR-Associated Complex for Anti-viral Defense (Cascade) complex; and
(d) at least one guide RNA (gRNA), wherein each gRNA is configured to hybridize to a portion of a target nucleic acid sequence.

2. The system of claim 1, wherein the one or more nucleic acids comprises one or more messenger RNAs, one or more vectors, or a combination thereof.

3. The system of claim 1 or claim 2, wherein Cas11, Cas3, and the two or more additional Cas proteins are encoded by a single nucleic acid.

4. The system of claim 1 or claim 2, wherein Cas11, Cas3, and the two or more additional Cas proteins are encoded by different nucleic acids.

5. The system of any of claims 1-4, wherein the guide RNA is encoded by a different nucleic acid than Cas 11, Cas3, the two or more additional Cas proteins, or a combination thereof.

6. The system of any of claims 1-3, wherein the guide RNA, Cas 11, Cas3, and the two or more additional Cas proteins are encoded by a single nucleic acid.

7. The system of any of claims 1-6, wherein at least one or all of Cas11, Cas3, and the two or more additional Cas proteins comprise a nuclear localization sequence or a tag.

8. The system of any of claims 1-7, wherein the two or more additional Cas proteins are selected from the group consisting of Cas5, Cas7, Cas6, and Cas8 or Cmx8.

9. The system of any of claims 1-8, wherein the engineered CRISPR-Cas system is derived from a Type 1 CRISPR-Cas system.

10. The system of claim 9, wherein the Type I CRISPR-Cas system is a Type I-B, a Type I-C, or a Type I-D system.

11. The system of any of claims 1-10, wherein the system comprises Cas11, Cas3, Cas5, Cas6, Cas7, and Cmx8.

12. The system of any of claims 1-10, wherein the system comprises Cas11, Cas3, Cas5, Cas6, Cas7, and Cas10.

13. The system of any of claims 1-10, wherein the system comprises Cas11, Cas3, Cas5, Cas7, and Cas8.

14. The system of claim 13, wherein the system is derived from Neisseria lactamica.

15. The system of any of claims 1-14, wherein the at least one gRNA is encoded in a CRISPR RNA (crRNA) array.

16. The system of any of claims 1-15, wherein the at least one gRNA comprises a non-naturally occurring gRNA.

17. The system of any of claims 1-16, wherein the system comprises two or more engineered CRISPR-Cas systems or one or more nucleic acids encoding two or more engineered (CRISPR-Cas) systems.

18. The system of claim 17, wherein the two or more engineered CRISPR-Cas systems are derived from different subtypes of Type I CRISPR-Cas systems.

19. The system of claim 17 or 18, wherein the two or more engineered CRISPR-Cas systems comprise two Type I CRISPR-Cas systems selected from the group consisting of: a Type I-B CRISPR-Cas system, a Type 1-C CRISPR-Cas system, and a Type I-D CRISPR-Cas system.

20. The system of any of claims 1-19, wherein the system further comprises at least one target nucleic acid.

21. The system of any of claims 1-20, wherein the system is a cell free system.

22. A composition comprising the system of any one of claims 1-20.

23. A eukaryotic cell comprising the system of any one of claims 1-20.

24. A method of altering a target nucleic acid sequence comprising contacting a target nucleic acid sequence with the system of any one of claims 1-20 or a composition of claim 22.

25. The method of claim 24, wherein altering a target nucleic acid sequence comprises deletion of the target nucleic acid sequence.

26. The method of claim 25, wherein the deletion is unidirectional.

27. The method of claim 25 or 26, wherein the deletion comprises from about 500 nucleotides to about 100,000 nucleotides.

28. The method of any of claims 25-27, wherein the deletion comprises from about 5,000 nucleotides to about 20,000 nucleotides.

29. The method of any of claims 24-28, wherein the target nucleic acid sequence encodes a gene product.

30. The method of any of claims 24-29, wherein the target nucleic acid sequence is in a cell.

31. The method of claim 30, wherein the cell is a eukaryotic cell.

32. The method of claim 30 or 31, wherein the cell is a mammalian cell.

33. The method of any of claims 30-32, wherein the cell is a human cell.

34. The method of any of claims 30-33, wherein the target nucleic acid sequence is a genomic DNA sequence.

35. The method of any of claims 30-34, wherein contacting a target nucleic acid sequence comprises introducing the system into the cell.

36. The method of claim 35, wherein introducing the system into the cell comprises administering the system to a subject.

37. The method of claim 36, wherein the subject is a human.

38. The method of claim 36 or 37, wherein the administering comprises in vivo administration.

39. The method of claim 36 or 37, wherein the administering comprises transplantation of ex vivo treated cells comprising the system.

40. Use of the system of any of claims 1-20 or a composition of claim 22 to alter a target nucleic acid sequence.
